(19) United States
(12) Patent Application Publication
(10) Pub. No.: US 2020/0176077 A1
(43) Pub. Date: Jun. 4, 2020

(54) STRUCTURAL PREDICTION OF PROTEINS

(71) Applicant: HUMAN LONGEVITY, INC., San Diego, CA (US)

(72) Inventors: Amalio TELENTI, San Diego, CA (US); Michael HICKS, San Diego, CA (US); Istvan BARTHA, San Diego, CA (US)

(21) Appl. No.: 16/638,104

(22) PCT Filed: Aug. 9, 2018
(86) PCT No.: PCT/US2018/046139

§ 371 Date: Feb. 10, 2020

Related U.S. Application Data
(60) Provisional application No. 62/543,253, filed on Aug. 9, 2017.

(51) Int. Cl. G16B 20/20; G16B 30/00; G06N 5/04

(52) U.S. Cl. CPC G16B 20/20; G06N 5/04; G16B 30/00

(57) ABSTRACT

Disclosed herein are methods and systems for determining regions, domains, or amino acid residues of proteins that are intolerant to mutation. Also disclosed are applications for visualizing intolerant proteins regions, domains, and amino acid residues.

### CROSS REFERENCE TO RELATED APPLICATIONS

This application claims the benefit to U.S. Provisional Application No. 62/543,253, filed Aug. 9, 2017, which is incorporated herein by reference in its entirety.

### BACKGROUND OF THE INVENTION

Recent large-scale sequencing projects of the human genome and exome detail the extent of genetic diversity in the human population. To date, there are over 4.7 million amino acid-changing (missense) variants reported in the human exome. Much attention has been directed to the association of variants with disease. However, these data also represent an unprecedented opportunity to characterize protein structure-function relationships in vivo. In particular, the pattern of distribution of genetic variants describes the functional limits to structural and functional modifications for a given protein. This information can be used to predict critical domains that would be informative for drug development and mechanism of action, including selectivity, lack of response, or toxicity.

### SUMMARY OF THE INVENTION

Protein structure-based methods are used in all stages of drug development, from target identification to lead optimization. Central to all structure-based discovery approaches is the knowledge of the three-dimensional (3D) structure of the target protein or complex because the structure and dynamics of the target determine which ligands it binds. A number of scoring approaches can measure the deleteriousness of genetic variants in a protein, a property that strongly correlates with both molecular functionality and pathogenicity. Scores may also consider interspecies conservation [GERP] to discover “constrained elements” indicative of putative functional elements. Recent sequencing efforts of human genomes and exomes provide a different level of spatial information through the saturation of proteins structures to derive human-specific constraints. The characterization of human-specific constraints and tolerance to genetic variation could be used to parse structural information to define active sites, but also to define functionally important topographically distinct sites that can support allosteric interactions. The presence of druggable, topographically distinct allosteric sites offers new advantages for the development of small molecules, antibodies, or apatmers to modulate protein function. Given the amount of data available current methods to determine amino acids, polypeptides, and domains from proteins intolerant to mutation are lacking. Current methods are underpowered, lack sufficient predictive capability, and require significant investments in in vitro experimental systems, which can be expensive and time-consuming.

The methods described herein for predicting the deleteriousness of any given mutant and portions of proteins that are intolerant to mutation improve upon the speed and accuracy of existing methods, and create rules, which can be extrapolated to all proteins, even ones with unknown structure, that have not had sufficient functional characterization. Using human genetic variation from nearly 140,000 human exomes and over 4700 x-ray protein structures and about 4000 homology models to model tolerance to amino acid changes in the 3D space of the human proteome (e.g., three-dimensional tolerance score or “3DTS”), yields precise functional prediction of structure-function at the protein level, and across dimerization or interaction surfaces. At an Angstrom resolution, the distribution of pathogenic variants in proteins complements existing analysis of deleteriousness of genetic variants. It is expected that this new dimension of 3D structural information supports understanding of mode of action, efficacy and toxicity of drugs, and facilitate drug design and target selection. The systems and methods of the disclosure are particularly useful in the identification of one or more intolerant site(s) in protein targets (preferably proteins targets that lack commercially available therapeutics, i.e., not yet druggable. Even in the context of druggable protein targets, the systems and methods of the disclosure may be used to identify additional intolerant sites in protein targets with commercially available therapeutics. Moreover, the systems and methods of the disclosure are particularly useful in the identification of potential sites of genetic resistance leading to drug inefficacy, for instance, identification of sites in protein targets that are susceptible to antibiotic resistance or resistance to anticancer drugs.

Described herein, in a certain aspect, is a method of determining a three-dimensional tolerance score (3DTS) for one or more amino acids of a protein comprising: (a) determining a global mutation rate, wherein the global mutation rate is an expected probability of any given nucleotide of the protein to vary; (b) determining a variant specific mutation rate for a missense mutation of a nucleic acid encoding the one or more amino acids of the protein, wherein the variant specific mutation rate is an observed probability of the missense mutation to occur in a sample nucleotide data set; and (c) determining the one or more amino acids of the protein as intolerant to variation if the variant specific mutation rate is less than the global mutation rate.

Further described herein are systems and methods for identifying drugability of a protein target comprising (a) determining a global mutation rate, wherein the global mutation rate is an expected probability of any given nucleotide of the protein to vary; (b) determining a variant specific mutation rate for a missense mutation of a nucleic acid encoding the one or more amino acids of the protein, wherein the variant specific mutation rate is an observed probability of the missense mutation to occur in a sample nucleotide data set; and (c) determining the one or more amino acids of the protein as intolerant to variation if the variant specific mutation rate is less than the global mutation rate, wherein if one or more amino acids of the protein are intolerant to the variation, then the protein is identified as being druggable. Under this embodiment, the protein may be druggable-naive (i.e., no commercial therapeutic exists that target the protein) or druggable-confirmed (one or more commercial therapeutic exists that target the protein).

Further described herein are systems and methods for identifying sites of genetic resistance to a drug (e.g., antibiotic, antibacterial, antifungal, anticancer drug) in a protein target comprising (a) determining a global mutation rate, wherein the global mutation rate is an expected probability of any given nucleotide of the protein to vary; (b) determining a variant specific mutation rate for a missense mutation of a nucleic acid encoding the one or more amino acids of the protein, wherein the variant specific mutation rate is an observed probability of the missense mutation to occur in a sample nucleotide data set; and (c) determining the one or more amino acids of the protein as tolerant to variation if the variant specific mutation rate is greater than the global mutation rate, wherein if one or more amino acids of the protein are tolerant to the variation, then the sites which comprise the amino acids that are tolerant to the variation are identified as conferring genetic resistance to the drug. Herein, amino acids that confer genetic resistance to the drug are tolerant to the variation (highly labile) so that when drug binds to the site, it does not cause drastic changes to the three-dimensional structure of the protein.

In addition to a global mutation rate based on the synonymous mutation rate relative to the reference genome-wide (“constant rate-synonymous” mutation), the systems and methods of the disclosure may incorporate two additional mutation rates: (1) variations based on the intergenic rate genome-wide; and (2) variations based on the intergenic rate specific to a chromosome. These additional types of mutation rates can be modulated within the heptameric context of a nucleotide (three nucleotides up and downstream of the reference nucleotide), which can be used to refine and improve (e.g., precision, sensitivity, accuracy, or specificity) of the methods.

In certain embodiments, the one or more amino acids of the protein comprise a plurality of amino acids. In certain embodiments, the plurality of amino acids comprises a protein feature or domain. In certain embodiments, the protein feature is selected from the list consisting of: an active site, a metal binding site, a chemical binding site, a DNA binding site, a nucleotide binding site, a zinc finger, a calcium binding site, a transmembrane domain, an intra membrane domain, a lipidation site, a glycosylation site, a phosphorylation site, a coiled-coil, an alpha helix, and a beta strand. In certain embodiments, the global mutation rate is the mutation rate of the nucleotides encoding the protein, an intronic sequence of the protein, a 3′ untranslated region of the protein, a 5′ untranslated region of the protein, or any combination thereof. In certain embodiments, the global mutation rate is the mutation rate for an entire human genome. In certain embodiments, the global mutation rate is between about 1×10⁻⁶ and 5×10⁻⁶. In certain embodiments, the global mutation rate is about 2.5×10⁻⁶. In certain embodiments, the sample nucleotide data set comprises at least 1,000 different nucleic acid sequences from at least 1,000 different individuals encoding the protein. In certain embodiments, the sample nucleotide data set comprises at least 10,000 different nucleic acid sequences from at least 10,000 different individuals encoding the protein. In certain embodiments, the nucleotide data set comprises DNA. In certain embodiments, one or more amino acids of the protein as intolerant to variation if the variant specific mutation rate is 2 times less than the global mutation rate. In certain embodiments, one or more amino acids of the protein as intolerant to variation if the variant specific mutation rate is 5 times less than the global mutation rate. In certain embodiments, the missense mutation is a hypothetical mutation. In certain embodiments, the method further comprises rendering a graphic representation of the protein with a visual indication of amino acids of the protein that are intolerant to variation. In certain embodiments, the graphic representation of the protein is three-dimensional. In certain embodiments, the graphic representation of the protein is rotatable around an x, y, or z axis. In certain embodiments, the graphic representation of the protein is reflectable across an x, y, or z axis. In one embodiment, the method provides for a binding site of a modulator that binds to any of the one or more amino acids of the protein that are intolerant to variation according to the method. In a certain embodiment, modulator is an antibody or antigen binding fragment thereof. In a certain embodiment, the modulator binds at a non-active or an allosteric site.

Described herein, in another aspect, is a computer-implemented system comprising a digital processing device comprising at least one processor, an operating system configured to perform executable instructions, a memory, and a computer program including instructions executable by the digital processing device to create an application for determining a three-dimensional tolerance score (3DTS) for one or more amino acids of a protein comprising:: (a) a software module determining a global mutation rate, wherein the global mutation rate is an expected probability of any given nucleotide of the protein to vary; (b) a software module determining a variant specific mutation rate for a missense mutation of a nucleic acid encoding the one or more amino acids of the protein, wherein the variant specific mutation rate is an observed probability of the missense mutation to occur in a sample nucleotide data set; and (c) a software module determining the one or more amino acids of the protein as intolerant to variation if the variant specific mutation rate is less than the global mutation rate. In certain embodiments, the one or more amino acids of the protein comprise a plurality of amino acids. In certain embodiments, the plurality of amino acids comprises a protein feature or domain. In certain embodiments, the protein feature or domain is selected from the list consisting of: an active site, a metal binding site, a chemical binding site, a DNA binding site, a nucleotide binding site, a zinc finger, a calcium binding site, a transmembrane domain, an intra membrane domain, a lipidation site, a glycosylation site, a phosphorylation site, a coiled-coil, an alpha helix, and a beta strand. In certain embodiments, the global mutation rate is the mutation rate of the nucleotides encoding the protein, an intronic sequence of the protein, a 3′ untranslated region of the protein, a 5′ untranslated region of the protein, or any combination thereof. In certain embodiments, the global mutation rate is the mutation rate for an entire human genome or for a protein-encoding portion of a human genome. In certain embodiments, the global mutation rate is between about 1×10⁻⁶ and 5×10⁻⁶. In certain embodiments, the global mutation rate is about 2.5×10⁻⁶. In certain embodiments, the sample nucleotide data set comprises at least 1,000 different nucleic acid sequences from at least 1,000 different individuals encoding the protein. In certain embodiments, the sample nucleotide data set comprises at least 10,000 different nucleic acid sequences from at least 10,000 different individuals encoding the protein. In certain embodiments, the nucleotide data set comprises DNA. In certain embodiments, one or more amino acids of the protein as intolerant to variation if the variant specific mutation rate is 2 times less than the global mutation rate. In certain embodiments, one or more amino acids of the protein as intolerant to variation if the variant specific mutation rate is 5 times less than the global mutation rate. In certain embodiments, the missense mutation is a hypothetical mutation. In certain embodiments, the system further comprises a software module rendering a graphic representation of the protein with a visual indication of amino acids of the protein that are intolerant to variation. In certain embodiments, the graphic representation of the protein is three-dimensional. In certain embodiments, the graphic representation of the protein is rotatable around an x, y, or z axis. In certain embodiments, the graphic representation of the protein is reflectable across an x, y, or z axis. In one embodiment, the system provides a list or file of binding sites for a modulator that binds to any of the one or more amino acids of the protein that are intolerant to variation according to the method employed by the system. In a certain embodiment, modulator is an antibody or antigen binding fragment thereof. In a certain embodiment, the modulator binds at a non-active or an allosteric site.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a user interface for a protein visualization tool.

FIGS. 2A and 2B show non-limiting examples of a method for determining and displaying protein portions that are intolerant to variation, missense variation data from genome and exome sequencing projects are mapped to 3D protein structures. (A) Features extracted from Uniprot are mapped to the 3D structures. Using these features as reference points, a 3D context is constructed and the corresponding genetic data are extracted. A 3D tolerance score (3DTS) is generated from this information. (B) The 3DTS values can be ranked and the corresponding tolerance ranks (or scores) can be projected back onto the 3D structure.

FIGS. 3A and 3B show distribution of 3DTS and median 3DTS for different feature types. (A) shows distribution of 3DTS values for 139,535 3D-sites for structures representing 4390 proteins. The 3DTS value at the 20^{th} percentile (3DTS<0.33), is used to define intolerant sites. (B) shows median 3DTS for a subset of feature types. The number of each feature type with a 3DTS value is shown above each column. The overall median across the structural proteome is represented by a horizontal dashed line.

FIGS. 4A-4F show correlation between in vitro functional data and 3DTS for various proteins. (A) shows a projection of the integrated functional scores described in Majithia et al. for each amino acid of PPARG, and (B) the scores averaged across the 3DTS-defined sites for a crystal structure 3dzy. The color scheme is chosen to match the one described in Majithia et al. (C) shows correlation between 3DTS and the 3D-site averaged in vitro scores for PPARG. (D) shows the distributions of Pearson r² values for all structures that cover at least 70% of the canonical isoform under four different 3DTS conditions: two different sets of 3D features and two different models of rate variation. (E) shows results of extensive evaluation of a large corpus of functional readouts for 1,026 proteins for which shallow mutational information is available. (F) shows a comparative bar graph for functional prediction of 3DTS with various published scores. These various scores trained under a range of assumptions, most commonly interspecies conservation, co-evolution, and pathogenicity. The results show that 3DTS performs comparably or better than the existing methods.

FIGS. 5A-5D show correlation between in vitro functional data and 3DTS for PPARG at different Angstrom resolutions. The 5 Å 3D site (r²=0.47) performs best relative to a linear site-approach as well as other 3D distances in a correlation analysis with in vitro data for PPARG The distances tested included (A, the linear site, no 3D context added; r²=0.099), (B, 3 Å r²=0.23), (C, 5 Å r²=0.47), and (D, 7 Å r²=0.44).

FIGS. 6A-6C show correlation between in vitro functional data and 3DTS for BRCA1. (A) shows a projection of the homology directed repair (HDR) scores described in Starita et al. (supra) averaged across amino acids, and (B) averaged across the 3DTS-defined sites. (C) shows correlation between 3DTS rank and the 3D-site averaged HDR scores.

FIG. 7 shows distance mapping of pathogenic variants shows the highest enrichment of pathogenic to benign variants to be near and within the most intolerant features defined by 3DTS.

FIGS. 8A-8C show raw counts and distances corresponding to FIG. 7. (A) pathogenic missense variants, (B) synonymous variants, and (C) common (allele frequency>1%) missense variants. Note that the first bin represents the information within the most intolerant 3D site and subsequent bins represent the counts only within each binned distance. The apparent “noisy” first few bins are due to biophysical constraints placed on inter-residue interactions (i.e., a minimum of about two Angstroms to find additional residues followed by more distance to identify subsequent residues, dependent upon orientation and residue types).

FIGS. 9A and 9B show (A) Binned 3DTS scores describing active sites, allosteric sites, drug ligand-binding sites, and background. The sum of each site type is 1. (B) Counts of tolerant and intolerant drug ligand-binding sites grouped by therapeutic area. Here, tolerant is defined as 3DTS>0.5, while intolerant is defined as described in the main text (3DTS<0.33).

FIGS. 10A-10D show histograms corresponding to the data presented in FIG. 9A for (A) active sites, (B) allosteric sites, (C) drug ligand-binding sites, and (D) background. The median of each plot is drawn as a vertical line.

FIGS. 11A-11F show a comparison of 3DTS, CADD score, and in vitro functional data for PPARG.

FIGS. 12A and 12B show that a 3DTS score improves standard methods to classify variants of unknown significance.

FIG. 13 shows a non-limiting example of a digital processing device; in this case, a device with one or more CPUs, a memory, a communication interface, and a display.

FIG. 14 shows a non-limiting example of a web/mobile application provision system; in this case, a system providing browser-based and/or native mobile user interfaces.

FIG. 15 shows a non-limiting example of a cloud-based web/mobile application provision system; in this case, a system comprising an elastically load balanced, auto-scaling web server and application server resources as well as synchronously replicated databases.

FIG. 16 shows a schematic chart of the workflow of the present disclosure.

FIG. 17 shows an embodiment of the specific workflow of the present disclosure.

FIG. 18 shows a schematic diagram of the system of the disclosure.

### DETAILED DESCRIPTION OF THE INVENTION

Described herein, is a method of determining a three-dimensional tolerance score (3DTS) for one or more amino acids of a protein comprising: determining a global mutation rate, wherein the global mutation rate is a probability of any given nucleotide of the protein to vary; determining a variant specific mutation rate for a missense mutation of a nucleic acid encoding the one or more amino acids of the protein, wherein the variant specific mutation rate is a probability of the missense mutation to occur in a sample nucleotide data set; determining the one or more amino acids of the protein as intolerant to variation if the variant specific mutation rate is less than the global mutation rate. In further specific embodiments, the (3DTS) score can be used to create an interactive display of a protein structure with amino acid residues intolerant to variation visually represented, using for example, highlighting, differential coloring (i.e., heat-mapping), bolding or thickening of the structure, indication by arrows, asterisks or some other character. The structure that is highlighted can be any structure that is able to adequately represent a protein in three dimensions such as a ribbon diagram or a space filling model. Alternatively, two-dimensional representation methods can be used such as a primary amino acid sequence represented by three-letter or single letterform. The interactive display can allow zooming, rotating, reflecting, or highlighting specific residues to get individual or contextual 3DTSs.

Described herein, in another aspect, is a computer-implemented system comprising a digital processing device comprising at least one processor, an operating system configured to perform executable instructions, a memory, and a computer program including instructions executable by the digital processing device to create an application for determining a three-dimensional tolerance score (3DTS) for one or more amino acids of a protein comprising: (a) a software module determining a global mutation rate, wherein the global mutation rate is an expected probability of any given nucleotide of the protein to vary; (b) a software module determining a variant specific mutation rate for a missense mutation of a nucleic acid encoding the one or more amino acids of the protein, wherein the variant specific mutation rate is an observed probability of the missense mutation to occur in a sample nucleotide data set; and (c) a software module determining the one or more amino acids of the protein as intolerant to variation if the variant specific mutation rate is less than the global mutation rate. Described herein, in another aspect, is a computer-implemented system comprising a digital processing device comprising at least one processor, an operating system configured to perform executable instructions, a memory, and a computer program including instructions executable by the digital processing device to create an application comprising: (a) a software module determining a global mutation rate, wherein the global mutation rate is an expected probability of any given nucleotide of the protein to vary; (b) a software module determining a variant specific mutation rate for a missense mutation of a nucleic acid encoding the one or more amino acids of the protein, wherein the variant specific mutation rate is an observed probability of the missense mutation to occur in a sample nucleotide data set; and (c) a software module determining the one or more amino acids of the protein as intolerant to variation if the variant specific mutation rate is less than the global mutation rate.

The systems and methods of the disclosure incorporate several features. One such feature is synonymous global mutation rate, defined as parameter p, which is the expected number of mutations at a locus assuming all mutations at a locus are neutral. In some embodiments, this is done by fitting the observed number of synonymous variants across all proteins to the expected number of synonymous variants by fixing s=1. A synonymous local mutation rate can also be used, which estimates heterogeneity across the genome, and is calculated as above, but is only evaluated on a single protein chain. In addition to these two methods of estimating a background/neutral mutation rate, a genome-wide intergenic variation rate or a chromosome-specific intergenic variation rate can be estimated from non-coding variation. This is done as above by determining the value p, which maximizes the likelihood function. Finally, a nucleotide-context dependent estimate can be used to estimate mutation rate heterogeneity. In this case, the 7-mer context which symmetrically spans the reference nucleotides. Then a maximum likelihood estimate specific for each heptamer is performed.

A second feature is additionally incorporated into the methods and systems of the present disclosure, which relates to a propensity towards missense variation. Herein, each reference nucleotide in a 3D-defined locus has 0, 1, 2, or 3 chances of a single nucleotide variation leading to a missense variant, defined as parameter b. This is determined based on the protein isoform for the 3D structure, the transcript encoding this protein isoform and the reference genome encoding this transcript for the locus. This parameter is normalized to 1 by dividing by 3 (i.e., 0/3, ⅓, ⅔, 3/3).

The systems and methods of the disclosure incorporate yet another parameter relating to the adjustment factor that is a proxy for the strength of purifying selection (parameter s). A value of s=1 signifies that the variants are as expected based on the background mutation rate (i.e., neutral effect) while a value of s=0 signifies that the locus is completely depleted of variation (i.e., intolerant). The systems and methods of the disclosure estimate parameter s based on various probabilistic and/or statistical outcome measurements.

In order to build the systems of the disclosure (3DTS), various steps and/or algorithms may be implemented. Herein, first, a locus in 3D protein space of interest is defined. In some embodiments, a 3D site may be defined as radius around a protein feature that may be 1, 2, 3, 4, 5, 6, 7, 8, 9, 10 (1 nm) or more Angstroms. The corresponding nucleotides (loci) defined by the 3D site are evaluated in this model. Next, variation data from genome/exome sequencing is used in the model. In some embodiments, the sequencing data of 140,000 individuals (e.g., human subjects) may be used in the model. Each nucleotide/locus that is defined as part of the loci (see above section on defining the locus) will have data, which is ordinarily represented in base units, e.g., adenosine (A), cytosine (C), guanine (G), thymine (T)(note: uracil or U may be used in place of thymine) for R individuals, where R may be all or a subset of the 140,000 individuals (depending on if there is a call or no call at that position). Each individual with a call will be either the reference nucleotide or an alternative nucleotide (e.g., a variant). Variants are treated as a separate Bernoulli trial.

In order to compute the 3D tolerance score (3DTS), a computational scheme is employed. Herein, the probability of observing a missense mutation at a locus l is defined by the background mutation rate (p), the propensity towards missense variation (b), and an adjustment factor that serves as a proxy for the strength of purifying selection (s): pₗ*sₗ*bₗ. The sequencing data for each person (i.e., each sample) is treated as a separate Bernoulli trial (i.e., presence or absence of a variant resulting in a missense mutation; see above). At a given locus, all parameters are the same across the samples, thus aggregating R samples yields a binomial distribution as the number of samples with a missense mutation at locus l. Using the Poisson approximation, the probability of observing at least one missense mutation in R samples in a single locus is l−exp(−pₗ*sₗ*bₗ*Rₗ).

Since each locus has different bₗ and Rₗ parameters, this is considered when aggregating over K loci (i.e., aggregating over the 3D feature). Thus, aggregating over these K>1 loci into a single value is the sum of Bernoulli trials of heterogeneous parameters, which may be approximated using a Poisson distribution following Le Cam's theorem. Thus, the final likelihood function of the model is: P (observed k variants in K loci among R samples|pₗ, s, bₗ)=Poi(k,Σₗ^{K} l−exp(−pₗ s bₗ Rₗ)).

As explained above, the bₗ parameter is a function of the genetic code, while the pₗ parameter is learned. Neutral sections of the genome are used to estimate this pₗ mutation rate parameter by setting s equal to 1 (assuming these sections of the genome are not deleterious) and the likelihood function under these constraints is maximized by incorporating these aspects.

Finally, to calculate the posterior mean on s with a uniform U(0,1) prior, a numerical integration (Gauss-Legendre quadrature and importance sampling) may be implemented. This posterior mean of s is defined as the 3D tolerance score (3DTS). The 3DTS score that may be used not only to identify whether a site is tolerant to variation but also to determine whether a site is druggable or resistant to drugs, or whether it is prone to allosteric modification, or whether it may confer genetic resistance leading to drug inefficacy (e.g., antibiotic resistance or resistance to anticancer drugs). In some embodiments, the 3D tolerance score is computed using a Bayesian inference, wherein, the mean of the posterior distribution, is the 3DTS value. That is, the mean of the probability distribution function of s given k observed variants in K loci among R samples given a background mutation rate p and a propensity towards missense variation b, is equal to the 3DTS (E[P(s|k)]==∫₀¹sP(s|k)ds). Herein, the likelihood function is expressed as L(k|s)=Poi(k,Σₗ^{K} l−exp(−pₗ*s*bₗ*Rₗ)) (equation 1); the prior function is expressed as P(s)=U(0,1) (equation 2); the probability of observing k variants (calculated using Gauss-Legendre quadrature; can also be calculated through importance sampling) is expressed as P(k)−∫₀¹L(k|s)* P(s)ds (equation 3); and the probability of the adjustment factor, s, given the observation of k variants is expressed as by the Bayes theorem

$$P ( s \vert k ) = \frac{L ( k \vert s ) * P ( s )}{P ( k )}$$

(i.e., equation 1*equation 2)/equation 3. The mean of the posterior (3DTS) is then computed as provided above.

In related embodiments, the disclosure provides systems comprising the following components: (a) a component or a module comprising 3-dimensional protein structure or model; (b) a component or a module comprising genome or exome sequencing data for several individuals that cover the 3D features of the protein; and (c) a computer-readable medium in which a program is stored for causing a computer to perform a method for determining tolerance to missense variation. The disclosure further includes methods for determining 3D tolerance score of a candidate protein via implementation of a plurality of steps comprising, (a) incorporating features based on the 3D protein structure or model; (b) incorporating features based on the sequencing data for a plurality of individuals that cover the 3D features of the protein; and (c) determining tolerance to missense variation based on features (a) and features (b).

With respect to component/features (a), preferably, the 3D protein features that are included in the protein structure or model is mappable to corresponding genomic data, e.g., via a database such as PDB. Here, 3D features may be defined based on: (i) a set of structural and/or functional annotated data available in the 3D structure itself; and/or (ii) 3D context around the annotated data set, which may be defined as those amino acids contained within a distance of a pre-defined radius, e.g., 1, 2,3,4,5,6,7,8,9,10 or more Angstroms from an amino acid or a motif/site of interest.

With respect to component/features (b), genome or exome sequencing data can be obtained in situ (by whole genome sequencing of a subject's sample) or from datasets, e.g., Broad Institute's genome aggregation database (gnomAD). Features extracted from protein databases such as UNIPROT may be mapped to the 3D structures (component/feature (a)). Using these features as reference points, a 3D context is constructed and the corresponding genetic data are extracted. Additional features that may be extracted from genome sequencing data include global mutation rates, regional mutational rate, intergenic variation, variation specific to a chromosome, or the like. A combination of genome and exome genetic data may also be used.

With regard to component/features (c), preferably, the computer readable medium stores a program for causing a computer to perform a method for determining tolerance to missense variation, which is defined by the mean of a posterior distribution, calculated through numerical integration using the Gauss-Legendre quadrature or estimated by importance sampling. Herein, the posterior distribution has several key features, including, use of a Bayes theorem, which combines a prior distribution and likelihood function; the prior distribution assumes all missense variants are tolerant and is set as a uniform distribution U(0,1); and the posterior distribution computes a likelihood function, which is defined as the sum of a series of Bernoulli trials and which may be estimated as a Poisson binomial distribution (detailed above).

Typically, the posterior distribution takes into consideration, one or more features in the genome or exome data comprising mutation rates, vis-a-vis, a background mutation rate, pₗ, which may be determined by fitting the observed number of presumed neutral variants to the expected number of neutral variants; and/or a propensity towards missense variation, bₗ, which may be determined for a specific protein isoform with a corresponding specific transcript with a corresponding specific reference genome for a corresponding specific locus. Preferably, both mutation rate features are employed in the posterior distribution. The posterior distribution further takes into consideration, an adjustment factor, s, which serves as a proxy for purifying selection. Typically, adjustment factor, s, is the parameter that is of interest in determining the probability, for the mean of the posterior distribution is indicative of the 3DTS.

### Certain Definitions

Unless otherwise defined, all technical terms used herein have the same meaning as commonly understood by one of ordinary skill in the art to which this invention belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

As used herein, the term “individual” refers to a human individual, unless otherwise specified.

As used herein, “protein” refers to polypeptides of biological origin, and incudes full-length proteins, fusion proteins, truncation mutants, proteins modified by epitope/affinity tags or fluorescent fusions that maintain at least one biological function attributable to the full-length unmodified version. In a certain embodiment, “protein” refers only to naturally occurring proteins unaltered by laboratory methods.

As used herein, the term “polypeptide” describes linear molecular chains of amino acids, including single chain proteins or their fragments, containing more than 30 amino acids. Polypeptides may further form oligomers consisting of at least two identical or different molecules. The corresponding higher order structures of such multimers are, correspondingly, termed homo- or heterodimers, homo- or heterotrimers etc. Homodimers, trimers etc. of fusion proteins giving rise or corresponding to enzymes also fall under the definition of the term “polypeptide.” Furthermore, peptidomimetics of such proteins/polypeptides where amino acid(s) and/or peptide bond(s) have been replaced by functional analogues are also encompassed by the invention. Such functional analogues include all known naturally occurring or synthetic amino acids other than the 20 gene-encoded (e.g., proteinogenic) amino acids, such as, e.g., selenocysteine or ketone-functionalized amino acids. The terms “polypeptide” also refer to naturally or synthetically modified polypeptides/proteins where the modification is effected e.g. by glycosylation, acetylation, phosphorylation and similar modifications, e.g., prenylation. The above applies mutatis mutandis also to the term “peptide” which as used herein describes a group of molecules consisting of up to 30 amino acids.

The term “proteome” as used herein refers to the entire set of proteins expressed by a genome, cell, tissue or organism. More specifically, the term proteome refers to the set of expressed proteins in a given type of cells or an organism at a given time under defined conditions. The term “proteome” also is used to refer to the collection of proteins in certain sub-cellular biological systems. A cellular proteome is the collection of proteins found in a particular cell type under a particular set of environmental conditions. For example, human proteome consists 92,179 proteins out of which 71,173 are splicing variants (Nucleic Acids Research 43 (D1): D204-D212. 2014). Eukaryotes, bacteria, archaea and viruses have on average 15,145, 3,200, 2,358 and 42 proteins respectively encoded in their genomes. See Kozlowski et al., Nucleic Acids Research 45 (D1): D1112-D1116, 2016.

As used herein, the term “lipid” relates to predominantly lipophilic/hydrophobic molecules, which may carry a polar headgroup. Lipids according to the invention include simple lipids such as hydrocarbons (triacontane, squalene, carotinoids), alcohols (wax alcohol, retinol, cholesterol, linear mono- or polyhydroxylated hydrocarbons, preferably with two to about 30 carbon atoms), ethers, fatty acids and esters such as mono-, di- and triacylgylcerols. Furthermore included are complex lipids such as lipoproteins, phospholipids and glycolipids. Phospholipids in turn comprise glycerophospholipids such as phosphatidic acid, lysophosphatidic acid, phosphatidylgylcerol, cardiolipin, lysobisphosphatidic acid, phosphatidylcholine, lysophosphatidylcholine, phosphatidylethanolamine, phosphatidylserine, phosphatidylinositol and phosphonolipids. Glycolipids include glycoglycerolipids such as mono- and digalactosyldiacylgylcerols and sulfoquinovosyldiacylgylcerol. The term “lipid” includes sphingomyelin glycosphingolipds and ceramides.

As used herein, the term “polynucleotide” includes DNA, such as cDNA or genomic DNA, and RNA. It is understood that the term “RNA” as used herein comprises all forms of RNA including mRNA, miRNA, siRNA, cRNA and the like. Further included are nucleic acid mimicking molecules known in the art such as synthetic or semisynthetic derivatives of DNA or RNA and mixed polymers, both sense and antisense strands. They may contain additional non-natural or derivatized nucleotide bases, as will be readily appreciated by those skilled in the art. Nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see, Braasch and Corey, Chemistry & Biology 8, 1-7, 2001). Typically, LNA is an RNA derivative in which the ribose ring is constrained by a methylene linkage between the 2′-oxygen and the 4′-carbon. A peptide nucleic acid (PNA) is a polyamide type of DNA analog. The monomeric units for the corresponding derivatives of adenine, guanine, thymine and cytosine are commercially available. PNA is a synthetic DNA-mimic with an amide backbone in place of the sugar-phosphate backbone of DNA or RNA. See Nielsen et al., Science 254:1497 (1991); and Egholm et al., Nature 365:666 (1993). The term includes PNA chimera comprising one or more PNA portions. The remainder of the chimeric molecule may comprise one or more DNA portions (PNA-DNA chimera) or one or more polypeptide portions (peptide-PNA chimera).

The term “derivatives” in conjunction with the above described PNAs, PNA chimera and peptide-DNA chimera relates to molecules wherein these molecules comprise one or more further groups or substituents different from PNA, polypeptides and DNA.

As used herein the term “small molecule” may include, a small organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the compound may be an inorganic compound. Inorganic compounds are derived from mineral sources and include all compounds without carbon atoms (except carbon dioxide, carbon monoxide and carbonates). Preferably, the small molecule has a molecular weight of less than about 10000 atomic mass units (amu), or less than about 5000 amu such as 1000 amu, 500 amu, and even less than about 250 amu. The size of a small molecule can be determined by methods well known in the art, e.g., mass spectrometry. In some embodiments, the small molecule has a molecular weight of less than about 10 KDa, preferably less than about 5 KDa, especially less than about 1 KDa (e.g., about 300 daltons to about 800 daltons). Small molecules may be designed, for example, in silico based on the crystal structure of potential drug targets, where sites presumably responsible for the biological activity and involved in the regulation of expression of genes identified herein, can be identified and verified in in vivo assays such as in vivo HTS (high-throughput screening) assays. Small molecules can be part of libraries that are commercially available, for example from CHEMBRIDGE Corp., San Diego, USA. In contrast, a “large molecule” has a molecular weight of greater than about 5 KDa, preferably greater than about 20 KDa, especially greater about 100 KDa.

As used herein, the term “drug” relates to compounds that have at least one biological and/or pharmacologic activity. Preferably, the drug is a compound used, a candidate compound intended for use in the treatment, cure, prevention, or diagnosis, used, or intended to be used to otherwise enhance physical or mental well-being.

As used herein, the term “prodrug” includes compounds that are generally not biologically and/or pharmacologically active. After administration, the prodrug is activated, typically in vivo by enzymatic or hydrolytic cleavage and converted to a biologically and/or pharmacologically active compound, which has the intended medical effect, i.e. is a drug that exhibits a biological and/or pharmacologic effect. Prodrugs are typically formed by chemical modification of biologically and/or pharmacologically active compounds. Conventional procedures for the selection and preparation of suitable prodrug derivatives are described, for example, in Design of Prodrugs, 1985.

As used herein, the term “second messengers” refers to molecules that relay signals from receptors on the cell surface to target molecules inside the cell, in the cytoplasma or nucleus. For example, second messengers are involved in the relay of the signals of hormones or growth factors and are involved in signal transduction cascades. Second messengers may be grouped in three basic groups: hydrophobic molecules (e.g., diacyglycerol, phosphatidylinositols), hydrophilic molecules (e.g., cAMP, cGMP, IP3, Ca2+) and gases (e.g., nictric oxide, carbon monoxide).

The term “metabolites” as used herein corresponds to its generally accepted meaning in the art, i.e. metabolites are intermediates and products of metabolism and may be grouped in primary (e.g., involved in growth, development and reproduction) and secondary metabolites.

As used herein, “aptamers” refer to molecules, e.g., oligonucleic acid or peptide molecules that bind a specific target molecule. Aptamers are usually created by selecting them from a large random sequence pool, but natural aptamers also exist in riboswitches. Further, they can be combined with ribozymes to self-cleave in the presence of their target molecule. More specifically, aptamers can be classified as DNA or RNA aptamers or peptide aptamers. Whereas the former consist of (usually short) strands of oligonucleotides, the latter consist of a short variable peptide domain, attached at both ends to a protein scaffold. Nucleic acid aptamers are nucleic acid species that may be engineered through repeated rounds of in vitro selection or equivalently, systematic evolution of ligands by exponential enrichment (SELEX) to bind to various molecular targets such as small molecules, proteins, nucleic acids, and even cells, tissues and organisms. Peptide aptamers consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein, which has good solubility properties, e.g., Thioredoxin-A. Peptide aptamer selection can be made using, e.g., yeast two-hybrid system.

As used herein, the term “oligosaccharides” refers to saccharide (e.g., sugar) polymers containing a small number of component sugars such as, e.g., at least (for each value) 3, 4, 5, 6, 7, 8, 9, 10, 11, 12, 13, 14, or at least 15 monosaccharides. They may be, e.g., O- or N-linked to amino acid side chains of polypeptides or to lipid moieties.

As used herein, an “antibody” includes whole antibodies and any antigen-binding fragment or a single chain thereof. Thus the antibody includes any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, which can be incorporated into an antibody of the present disclosure. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments to a preselected target. Examples of binding fragments encompassed within the term “antigen binding portion” of an antibody include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 242:423-426, 1988; Huston et al., PNAS USA, 85:5879-5883, 1988), including diabodies. Such single chain antibodies and diabodies are also intended to be encompassed within the term “antigen-binding fragment” of an antibody. These antibody fragments are obtained using conventional techniques known to those with skill in the art, and the fragments are screened for utility in the same manner, as are intact antibodies. Conversely, libraries of scFv constructs can be used to screen for antigen binding capability and then, using conventional techniques, spliced to other DNA encoding human germline gene sequences. One example of such a library is the “HuCAL: Human Combinatorial Antibody Library” (Knappik et al., J Mol Biol., 296(1):57-86, 2000). Antibodies may be obtained using immunization of a host, e.g., rabbit or Guinea pig, and obtaining the blood or sera thereof. Alternately, hybridoma technique, trioma technique, the human B-cell hybridoma technique (Kozbor et al., 1983; Li et al., 2006) may be used. Furthermore, recombinant antibodies may be obtained from monoclonal antibodies or can be prepared de novo using various display methods such as phage, ribosomal, mRNA, or cell display. A suitable system for the expression of the recombinant (humanized) antibodies or fragments thereof may be selected from, for example, bacteria, yeast, insects, mammalian cell lines or transgenic animals or plants (see, e.g., U.S. Pat. No. 6,080,560; Holliger and Hudson, 2005). Further, techniques described for the production of single chain antibodies (see, U.S. Pat. No. 4,946,778) can be adapted to produce single chain antibodies specific for the targets of the disclosure. Surface plasmon resonance as employed in the BIACORE system can be used to characterize the efficiency of phage antibodies for further optimization.

As used herein, the term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity, which have variable, and constant regions derived from human germline immunoglobulin sequences.

An “interaction” as used in accordance with the invention is either a direct physical interaction, also referred to as “binding”, or an indirect interaction mediated by other constituents that may or may not be endogenous components of the cell. As defined in the main embodiment, said reaction, preferably binding occurs within said cell. In other words, the reaction, preferably binding to be determined, occurs or may occur between said potential intracellular interaction, preferably binding partner and the intracellular domain of said receptor.

As used herein, the term “determining an interaction” includes determining presence or absence of a given interaction, detecting whether a previously unknown interaction occurs, quantifying interactions, wherein said interactions may include known as well as previously unknown interactions. The method according to the invention also extends to observing an interaction, wherein said observing may also include observing or monitoring over time and/or at more than one location, preferably locations within a site of interest, e.g., active site, allosteric site, epitope, interacting motif or domain. Methods of quantifying such interactions include both dry science (e.g., use of computational software) as well as wet science (e.g., determination of binding kinetics such as dissociation constants or KD using purified, recombinant proteins) or semi-wet science (e.g., using BIACORE assays). The interaction to be determined is preferably binding.

As used herein, the term “protein reaction” means that a target protein (e.g., receptor, enzyme, hormone, growth factor) changes its structure in response to changes in its environment, e.g., in the presence or absence of an activator, inhibitor or a modulator. A “protein reaction” may also be induced by many factors, such as a change in temperature, pH, voltage, ion concentration, phosphorylation, or the binding of a ligand. One type of protein reaction is a “conformational change”. If the conformational change alters the binding affinity of the chimeric transmembrane receptor to an intracellular binding partner, the change in the interaction strength may be determined as described above. The protein reaction of the chimeric transmembrane receptor may also include proteolytic cleavage.

As used herein, the term “high affinity” for a binding partner (e.g., ligand or antibody) refers to an molecule having a KD of 10⁻⁶ M or less, more preferably 10⁻⁸M or less and even more preferably 10⁻⁹M or less, e.g., 10⁻¹⁰ M or even 10⁻¹¹ M. The term may be molecule-specific. For example, “high affinity” binding in the context of an IgM isotype may to an antibody having a K_{D} of 10⁻⁷ M or less, more preferably 10⁻⁸ M or less, e.g., 10⁻⁹ M.

As used herein, the terms “dissociation constant,” “K_{dis},” “K_{D},” “Kd” refer to the dissociation rate of a particular interaction, e.g., ligand-receptor, drug-enzyme, antibody-antigen interaction, which is typically a ratio of the rate of dissociation (k₂), also called the “off-rate (k_{off})”, to the rate of association rate (k₁) or “on-rate (kₒₙ)”. Thus, K_{D} equals k2/k1 or k_{off}/kₒₙ and is expressed as a molar concentration (M). It follows that the smaller Kd, the stronger the binding. Therefore, 10⁻⁶M (or 1 μM) indicates weak binding compared to 10⁻⁹M (or 1 nM).

The terms “specifically binds” and “specific binding” when made in reference to the binding of two molecules, e.g., antibody and an antigen, refer to an interaction which is dependent upon the presence of a particular structure on the molecule(s). For example, if an antibody is specific for epitope “A” on the molecule, then the presence of a protein containing epitope A (or free, unlabeled A) in a reaction containing labeled “A” and the antibody will reduce the amount of labeled A bound to the antibody. In one embodiment, the level of binding of a molecule (e.g., drug, antibody, ligand) to its binding partner (e.g., enzyme, antigen, receptor) is determined using the “IC₅₀” i.e., “half maximal inhibitory concentration” that refers to the concentration of a substance (e.g., inhibitor, antagonist, etc.) that produces a 50% inhibition of a given biological process, or a component of a process (e.g., binding between drug and enzyme and/or the resulting biological effect, e.g., inhibition of enzyme activity). It is commonly used as a measure of an antagonist substance's potency.

As used herein, “specific binding” in the context of an antibody-antigen interaction refers to binding with a dissociation constant (K_{D}) of about 10⁻⁷M or less to the antigen (e.g., a receptor such as Her2), preferably 10⁻⁸M or less and even more preferably 10⁻⁹M or less, e.g., 10⁻¹⁰ M or even 10⁻11 M. Additionally, the antibody may bind to the antigen with a K_{D} that is at least about 3-fold, 4-fold, or 5-fold less than its &for binding to a non-specific antigen (e.g., BSA, casein, or a random polypeptide having a sequence that is not present in the particular antigen (e.g., a receptor such as Her2)). As used herein “highly specific” binding means that the relative K_{D} of the antibody for the specific target epitope is at least 10-fold, at least 20-fold, e.g., about 50-fold less than the K_{D} for binding that antibody to other ligands (e.g., BSA, casein, or a random polypeptide).

As used herein, the term “pharmaceutically acceptable” means a molecule or a material that is not biologically or otherwise undesirable, i.e., the molecule or the material can be administered to a subject without causing any undesirable biological effects such as toxicity.

As used herein, the term “carrier” denotes buffers, adjuvants, dispersing agents, diluents, and the like. For instance, the peptides or compounds of the disclosure can be formulated for administration in a pharmaceutical carrier in accordance with known techniques. See, e.g., Remington, The Science & Practice of Pharmacy (9^{th} Ed., 1995). In the manufacture of a pharmaceutical formulation according to the disclosure, the peptide or the compound (including the physiologically acceptable salts thereof) is typically admixed with, inter alia, an acceptable carrier. The carrier can be a solid or a liquid, or both, and is preferably formulated with the peptide or the compound as a unit-dose formulation, for example, a tablet, which can contain from about 0.01 or 0.5% to about 95% or 99%, particularly from about 1% to about 50%, and especially from about 2% to about 20% by weight of the peptide or the compound. One or more peptides or compounds can be incorporated in the formulations of the disclosure, which can be prepared by any of the well-known techniques of pharmacy.

As used herein, the term “culture,” refers to any sample or specimen which is suspected of containing one or more microorganisms or cells. “Pure cultures” are cultures in which the cells or organisms are only of a particular species or genus. This is in contrast to “mixed cultures,” wherein more than one genus or species of microorganism or cell are present.

As used herein, the terms “treat,” “treating,” or “treatment of,” refers to reduction of severity of a condition or at least partially improvement or modification thereof, e.g., via complete or partial alleviation, mitigation or decrease in at least one clinical symptom of the condition, e.g., cancer.

As used herein, the term “administering” is used in the broadest sense as giving or providing to a subject in need of the treatment, a composition such as a drug. For instance, in the pharmaceutical sense, “administering” means applying as a remedy, such as by the placement of a drug in a manner in which such molecule would be received, e.g., intravenous, oral, topical, buccal (e.g., sub-lingual), vaginal, parenteral (e.g., subcutaneous; intramuscular including skeletal muscle, cardiac muscle, diaphragm muscle and smooth muscle; intradermal; intravenous; or intraperitoneal), topical (i.e., both skin and mucosal surfaces), intranasal, transdermal, intraarticular, intrathecal, inhalation, intraportal delivery, organ injection (e.g., eye or blood, etc.), or ex vivo (e.g., via immunoapheresis).

As used herein, “contacting” means that the composition comprising the active ingredient is introduced into a sample containing a target, e.g., a protein target, a cell target, in an appropriate environment, e.g., within a software application, a BIACORE system, a test tube, flask, tissue culture, chip, array, plate, microplate, capillary, or the like, and incubated at a temperature and time sufficient to permit binding (e.g., target binding to an unknown binding partner) or vice versa (e.g., a binding partner binding to an unknown target). In the in vivo context, “contacting” means that the therapeutic or diagnostic molecule is introduced into a patient or a subject for the treatment of a disease, and the molecule is allowed to come in contact with the patient's target tissue, e.g., blood tissue, in vivo or ex vivo.

As used herein, the term “therapeutically effective amount” refers to an amount that provides some improvement or benefit to the subject. Alternatively stated, a “therapeutically effective” amount is an amount that will provide some alleviation, mitigation, or decrease in at least one clinical symptom in the subject. Methods for determining therapeutically effective amount of the therapeutic molecules, e.g., anticancer agents or antibodies, are known in the art, and may include in vitro assays or in vivo pharmacological assays.

As used herein, the term “modulate,” with reference to an interaction between a target and its partner means to regulate positively or negatively the normal biological function of a target. Thus, the term modulate can be used to refer to an increase, decrease, masking, altering, overriding or restoring the normal functioning of a target. A modulator can be an agonist, a partial agonist, or an antagonist, a cofactor, an allosteric activator or inhibitor or the like.

As used herein, the term “inhibit” refers to reduction in the amount, levels, density, turnover, association, dissociation, activity, signaling, or any other feature associated with a target agent, e.g., an enzyme or a receptor or an antigen.

As used herein, the term “subject” means an individual. In one aspect, a subject is a mammal, e.g., a human or a non-human primate. Non-human primates include marmosets, monkeys, chimpanzees, gorillas, orangutans, and gibbons. Subjects include domesticated animals, such as cats, dogs, etc., livestock (e.g., llama, horses, cows), wild animals (e.g., deer, elk, moose, etc.,), laboratory animals (e.g., mouse, rabbit, rat, gerbil, guinea pig, etc.) and avian species (e.g., chickens, turkeys, ducks, etc.). Preferably, the subject is a human, especially, a human patient.

As used herein, the term “tumor” is used to denote neoplastic growth which may be benign (e.g., a tumor which does not form metastases and destroy adjacent normal tissue) or malignant/cancer (e.g., a tumor that invades surrounding tissues, and is usually capable of producing metastases, may recur after attempted removal, and is likely to cause death of the host unless adequately treated). See Steadman's Medical Dictionary, 28^{th} Ed Williams & Wilkins, Baltimore, Md. (2005).

As used herein, the term “detecting,” refers to the process of determining a value or set of values associated with a sample by measurement of one or more parameters in a sample, and may further comprise comparing a test sample against reference sample. In accordance with the present disclosure, the detection of binding between a target and its binding partner may include identification, assaying, measuring and/or quantifying one or more interactions between a site in a target, e.g., active site or an allosteric site in an enzyme; an epitope in an antigen, or a ligand-binding site in a receptor.

As used herein, a “detectable label” is a moiety, the presence of which can be ascertained directly or indirectly. Generally, detection of the label involves the creation of a detectable signal such as for example an emission of energy. The label may be of a chemical, peptide or nucleic acid nature although it is not so limited. The nature of label used will depend on a variety of factors, including the nature of the analysis being conducted, the type of the energy source and detector used and the type of polymer, analyte, probe and primary and secondary analyte-specific binding partners. The label should be sterically and chemically compatible with the constituents to which it is bound. The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.). Generally the detectable label can be selected from the group consisting of directly detectable labels such as a fluorescent molecule ((e.g., fluorescein, rhodamine, tetramethylrhodamine, R-phycoerythrin, Cy-3, Cy-5, Cy-7) or indirectly detectable labels such as an enzyme (e.g., alkaline phosphatase, horseradish peroxidase, p-galactosidase, glucoamylase, lysozyme, luciferases such as firefly luciferase and bacterial luciferase).

As used herein, the term “specific detection” refers to level of detection of a particular target (“signal”) over other non-targets (“noise”). Specific detection is achieved when the signal-to-noise for the detection is at least 0.6-fold, 0.7-fold, 0.8-fold, 0.9-fold, 1-fold, 1.5-fold, 2-fold (e.g., 100% increase), 3-fold, 5-fold, 10-fold, 20-fold, 50-fold, 70-fold, 100-fold, or more.

As used herein the term “signal” is used in reference to an indicator that a reaction has occurred, for example, binding of antibody to antigen. It is contemplated that signals in the form of radioactivity, fluorescence reactions, luminescent and enzymatic reactions will be used with the present disclosure. The signal may be assessed quantitatively as well as qualitatively. As used herein the term “signal intensity” refers to magnitude of the signal strength wherein the intensity correlates with the amount of reaction substrate.

As used herein, the term “cell” refers to a basic unit of life. The term “biological cell” include eukaryotic cells, plant cells, animal cells, such as mammalian cells, insect cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immune cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, e.g., from a human, a mouse, a rat, a horse, a goat, a sheep, a cow, a primate, etc.

As used herein, the term “sample” refers to a composition that is obtained or derived from a subject of interest that contains a cellular and/or other molecular entity that is to be characterized and/or identified, for example based on physical, biochemical, chemical and/or physiological characteristics. As used herein a “biological sample” is a substance obtained from the subject's body. The particular “biological sample” selected will vary based on the disorder the patient is suspected of having and, accordingly, which biological sample is most likely to contain the analyte. The source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, and tissue culture medium, as well as tissue extracts such as homogenized tissue, tumor tissue, and cellular extracts. Samples further include biological samples that have been manipulated in any way after their procurement, such as by treatment with reagents such as a histological sample. Preferably, the sample is obtained from blood or blood components, including, e.g., whole blood, plasma, serum, lymph, and the like.

As used herein, “biological data” can refer to any data derived from measuring biological conditions of human, animals or other biological organisms including microorganisms, viruses, plants and other living organisms. The measurements may be made by any tests, assays or observations that are known to physicians, scientists, diagnosticians, or the like. Biological data can include, but is not limited to, clinical tests and observations, physical and chemical measurements, genomic determinations, genomic sequencing data, exome sequencing data, proteomic determinations, drug levels, hormonal and immunological tests, neurochemical or neurophysical measurements, mineral and vitamin level determinations, genetic and familial histories, and other determinations that may give insight into the state of the individual or individuals that are undergoing testing. As used herein, “phenotypic data” refer to data about phenotypes.

As used herein, the term “marker” refers to a characteristic that can be objectively measured as an indicator of normal biological processes, pathogenic processes or a pharmacological response to a therapeutic intervention, e.g., treatment with an anti-cancer agent. Representative types of markers include, for example, molecular changes in the structure (e.g., sequence) or number of the marker, comprising, e.g., gene mutations, gene duplications, or a plurality of differences, such as somatic alterations in DNA, copy number variations, tandem repeats, or a combination thereof.

As used herein the term “exomic marker” refers to a polynucleotide sequence that is translated into a protein product. As is understood in the art, the exome is the part of the genome formed by exons, the sequences which when transcribed remain within the mature RNA after introns are removed by RNA splicing. It comprises all DNA that is transcribed into mature RNA in cells of any type. In contrast, the transcriptome comprises RNA that has been transcribed only in a specific cell population. The exome of the human genome consists of roughly 180,000 exons constituting about 1% of the total genome, or about 30 megabases of DNA (Ng et al., Nature, 461, 272-276, 2009) Though comprising a very small fraction of the genome, mutations in the exome are thought to harbor 85% of mutations that have a large effect on disease (Choi et al., PNAS USA, 106, 19096-19101, 2009). Exome sequencing has proved to be an efficient strategy to determine the genetic basis of more than two dozen Mendelian or single gene disorders (Bamshad et al., Nat Rev Genet., 12, 745-755, 2011).

The term “target” refers to any molecule of interest. Preferably, the target is an informational molecule such as, e.g., a protein and/or an mRNA encoded by a genomic sequence, as well as to that genomic sequence itself. An “agent” is a molecule that interacts with the target, e.g., via specific binding. Non-limiting examples of target-agent pairs include, e.g., enzyme-enzyme modulators (e.g., kinase-kinase inhibitors; phosphatase-phosphatase activators; histone deacylase (HDAC)-HDAC modulators); signaling pathway modulators (e.g., sonic hedgehog (SHH)-SHH modulators; G-protein coupled receptors (GPCR)-GPCR modulators); receptor-ligands (e.g., growth factor receptors and ligands thereof such as EGFR, HGF, VEGF, KIT; hormone receptors and ligands thereof such as estrogen receptor, androgen receptor, FSH receptor, thyroid hormone receptor, vitamin D receptor; small hormone receptors and ligands thereof such as dopamine receptor, serotonin receptor, histamine receptor)); neuropeptides and receptors thereof (e.g., CRH, GHRH, LHRH, neurokinin b, neuropeptide K and substance P; opioid peptides such as b-endorphin, dynorphin and met- and leu-enkephalin; NPY and related peptides such as neuropeptide tyrosine (NPY), pancreatic polypeptide and peptide tyrosine-tyrosine (PYY); VIP-glucagon family members such as glucogen-like peptide-1 (GLP-1), peptide histidine isoleucine (PHI), pituitary adenylate cyclase activating peptide (PACAP) and vasoactive intestinal polypeptide (VIP); BNP and isoforms thereof); ionophores (e.g., K+ ionophores, Ca2+ ionophores, Ba2+ ionophores, HCO3− ionophores, NO3 ionophores); ion channel modulators (e.g., K+ channel agonist, Na+ channel blocker, Ca2+ channel blocker); adenosine receptor modulators (e.g., modulators of A1, A2A, A2B, or A3 receptors); complement system proteins (e.g., C1, C2, C3, C4, C5; preferably C5); steroid receptors and steroids (e.g., 3-Ketosteroid receptors which interact with cortisol, aldosterone, progesterone, testesterone; retinoic receptors which interact with retinoids; PPAR-β/δ which interact with fatty acids, prostaglandins; pregnane X receptors which interact with xenobiotics); and gamma secretase inhibitors, including inhibitors of polypeptide components thereof, e.g., presenilin (PS), nicastrin (NCT), PEN-2 and APH-1. Representative types of modulators for the various aforementioned targets are disclosed in U.S. Pub. No. 2016/0220580, which is incorporated by reference herein in its entirety. Preferably, the target molecules and the agents that interact with the targets are disclosed in Table 2.

The term “cancer” as used herein refers to various sarcoma and carcinoma and includes solid cancer and hematopoietic cancer. The solid cancer as referred to herein includes, for example, brain cancer, cervicocerebral cancer, esophageal cancer, thyroid cancer, small cell lung cancer, non-small cell lung cancer, breast cancer, endometrial cancer, lung cancer, stomach cancer, gallbladder/bile duct cancer, liver cancer, pancreatic cancer, colon cancer, rectal cancer, ovarian cancer, choriocarcinoma, uterus body cancer, uterocervical cancer, renal pelvis/ureter cancer, bladder cancer, prostate cancer, penis cancer, testicles cancer, fetal cancer, Wilms' tumor, skin cancer, malignant melanoma, neuroblastoma, osteosarcoma, Ewing's tumor, soft part sarcoma. On the other hand, the hematopoietic cancer includes, for example, acute leukemia, chronic lymphatic leukemia, chronic myelocytic leukemia, polycythemia vera, malignant lymphoma, multiple myeloma, Hodgkin's lymphoma, non-Hodgkin's lymphoma.

The target molecules of the disclosure include bacterial, yeast fungal, or mammalian (e.g., human) proteins that can be targeted with antibacterial, anti-yeast, anti-fungal or therapeutic agents.

The term “antibiotic” as used herein refers to any molecule that produces effects adverse to the normal biological functions of the cell, tissue or organism including death or destruction and prevention of the division, growth, proliferation or differentiation of the biological system when contacted with said molecule. While presently not desiring to be bound by mechanism or theory, it is believed that the effective antibiotics are those which resist hydrolysis by an enzyme. Preferably, antibiotics include glycopeptide antibiotics and β-lactam antibiotics. Glycoside antibiotics include, e.g., streptomycin, neomycin, gentamicin, and vancomycin. β-lactam antibiotics include, e.g., penicillin, ampicillin, and amoxicillin. Other examples are cephalosporin β-lactams, e.g., cephalexin, cefadroxil, cephamycin, and latamoxef.

The term “anticancer agent” as used herein refers to any molecule that produce effects adverse to the normal biological functions of a cancer cell, for example, an anticancer agent selected from the group consisting of anticancer alkylating agents, anticancer antimetabolites, anticancer antibiotics, plant-derived anticancer agents, anticancer platinum coordination compounds, anticancer camptothecin derivatives, anticancer tyrosine kinase inhibitors, monoclonal antibodies, interferons, biological response modifiers, mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine and goesrelin as well as pharmaceutically acceptable salt(s) or ester(s) thereof.

As used herein, the term “variation” refers to a change or deviation. In reference to nucleic acid, a variation refers to a difference(s) or a change(s) between DNA nucleotide sequences, including differences in copy number (CNVs). This actual difference in nucleotides between DNA sequences may be an SNP, and/or a change in a DNA sequence, e.g., fusion, deletion, addition, repeats, etc., observed when a sequence is compared to a reference, such as, e.g., germline DNA (gDNA) or a reference human genome HG38 sequence. Preferably, the variation refers to difference between sample sequence and a control DNA sequence, such as when a sample sequence is compared to reference HG38 sequence; when a sample sequence is compared to gDNA. Differences identified in both gDNA and cfDNA are considered “constitutional” and may be ignored.

As used herein, the term “altered” in reference to a gene product, e.g., mRNA (or the DNA equivalent thereof or the complement of the mRNA or the DNA equivalent) or a polypeptide encoded by the mRNA or the DNA equivalent, refers to a difference in the structure (e.g., nucleic acid sequence or amino acid sequence), level, activity, or function of the gene product compared to a control. Preferably, the altered gene product comprises missense mutations or loss-of-function (LoF) mutations.

As used herein, the term “genetic variant” or “variant” refers to a nucleotide sequence in which the sequence differs from the sequence most prevalent in a population, for example by one nucleotide, in the case of the SNPs described herein. For example, some variations or substitutions in a nucleotide sequence alter a codon so that a different amino acid is encoded resulting in a genetic variant polypeptide. The term “genetic variant,” can also refer to a polypeptide in which the sequence differs from the sequence most prevalent in a population at a position that does not change the amino acid sequence of the encoded polypeptide (i.e., a conserved change). Genetic variant polypeptides can be encoded by a risk haplotype, encoded by a protective haplotype, or can be encoded by a neutral haplotype. Genetic variant polypeptides can be associated with risk, associated with protection, or can be neutral.

Non-limiting examples of genetic variants include frameshift, stop gained, start lost, splice acceptor, splice donor, stop lost, inframe indel, missense, splice region, synonymous and copy number variants. Non-limiting types of copy number variants include deletions and duplications.

As used herein, “genetic variant data” refer to data obtained by identifying allelic variants in a subject's nucleic acid, relative to a reference nucleic acid sequence. The term “genetic variant data” also encompasses data that represent the predicted effect of a variant on the biochemical structure/function of the polypeptide encoded by the variant gene.

In contrast to a variant, a “wild-type” generally refers to a biomolecule (e.g., polypeptide or polynucleotide) comprising a structure (e.g., an amino acid sequence or a polynucleotide sequence) of a naturally occurring, non-mutated biomolecule.

Preferably, the exomic marker or the genetic marker includes variant nucleic acids, e.g., mutations, SNPs, CNVs, STRs, or a combination thereof compared to a reference sample. Particularly, the variations are in the coding region of the nucleic acids, especially in the exomes. The variant nucleic acids preferably encode for an altered protein product, e.g., a protein product whose amino acid composition or length or both is different from a reference (e.g., wild-type) polypeptide product.

As used herein, the term “missense mutation” refers to a change in the DNA sequence that changes a codon in the MRNA that is normally translated as one amino acid into a codon that is translated as a different amino acid. For example, a mutation in which the ‘C’ in 5′-TCA is changed to ‘T’ (UCA to UUA in the mRNA) is a missense mutation. The serine encoded by the TCA codon would be replaced by leucine, the amino acid encoded by the TTA (UUA) codon, when the protein is synthesized in the cell. Some but not all missense mutations result in a non-functional gene-product. Some missense mutations may also result in a gain of function. A selection method may be used to find those missense mutations that substantially affect the protein function.

As used herein, the term “loss-of-function (LoF) mutation” or “inactivating mutation” refers to mutations which result in partial or complete inactivation of the gene product. The term includes “amorphic mutation” which refers to instances wherein an allele has a complete loss of function (null allele). Phenotypes associated with amorphic mutations are most often recessive. Exceptions are when the organism is haploid, or when the reduced dosage of a normal gene product is not enough for a normal phenotype (termed haploinsufficiency). In contrast “gain-of-function (GoF) mutations” or “activating mutations” refers to mutations which enhance activity of the protein product or which result in a wholly different (and abnormal) activity of the protein. When the new allele is created containing a GoF mutation, a heterozygote containing the newly created allele as well as the original allele will express the new allele; genetically this defines the mutations as dominant phenotypes.

In some embodiments, the missense mutations give rise to dominant negative mutations (DN). The term “dominant negative mutation” or “antimorphic mutation” refers to a mutation which results in an altered gene product that acts antagonistically to the wild-type allele. These mutations usually result in an altered molecular function (often inactive) and are characterized by a dominant or semi-dominant phenotype. In humans, dominant negative mutations have been implicated in cancer (e.g., mutations in genes p53, ATM, CEBPA and PPARy).

As used herein, the term “germline DNA” or “gDNA” refers to DNA isolated or extracted from a subject's germline cells, e.g., peripheral mononuclear blood cells, including lymphocytes that are in turn obtained from circulating blood.

The term “control,” as used herein, refers to a reference for a test sample, such as control DNA isolated from peripheral mononuclear blood cells and lymphocytes, where these cells are not cancer cells, and the like. A “reference sample,” as used herein, refers to a sample of tissue or cells that may or may not have cancer that are used for comparisons. Thus a “reference” sample thereby provides a basis to which another sample, for example plasma sample containing markers, e.g., exomic markers can be compared. In contrast, a “test sample” refers to a sample compared to a reference sample or control sample. In some embodiments, the reference sample or control may comprise a reference assembly.

The term “reference assembly” refers to a digital nucleic acid sequence database, such as the human genome (HG38) database containing HG38 assembly sequences. The gateway can be accessed through the Human (Homo sapiens) University of California Santa Cruz Genome Browser Gateway via the web at genome(dot)ucsc(dot)edu. Alternately, the reference assembly may refer to the Genome Reference Consortium's Human Genomic Assembly (Build #38; Assembled: June, 2017), which is accessible on the internet via the U.S. NCBI website.

In some embodiments, the reference assembly comprises an “exome assembly” or a “transcriptome assembly.” As the name suggests, these refer to a digital nucleic acid sequence database containing the exome or the transcriptome assembly sequences, respectively. In some embodiments, these databases are assembled using a reference assembly such as HG38 assembly sequences. Alternately, institutional exome assemblies can be utilized. An example is Garvan Institute of Medical Research whole-exome sequence data, which is utilized by Illumina's SEQMAN NGEN 12.2 to analyze Illumina-based sequence data.

As used herein, the term “sequencing” or “sequence” as a verb refers to a process whereby the nucleotide sequence of DNA, or order of nucleotides, is determined, such as a nucleotide order AGTCC, etc. The term “sequence” as a noun refers to the actual nucleotide sequence obtained from sequencing; for example, DNA having the sequence AGTCC. Wherein the “sequence” is provided and/or received in digital form, e.g., in a disk or remotely via a server, “sequencing” may refer to a collection of DNA that is propagated, manipulated and/or analyzed using the methods and/or systems of the disclosure.

The phrase “sequencing run” refers to any step or portion of a sequencing experiment performed to determine some information relating to at least one biomolecule (e.g., nucleic acid molecule).

As used herein the term “whole exome sequencing” refers to selective sequencing of coding regions of the DNA genome. The targeted exome is usually the portion of the DNA that translate into proteins, however regions of the exome that do not translate into proteins may also be included within the sequence. The robust approach to sequencing the complete coding region (exome) can be clinically relevant in genetic diagnosis due to the current understanding of functional consequences in sequence variation, by identifying the functional variation that is responsible for both Mendelian and common diseases without the high costs associated with a high coverage whole-genome sequencing while maintaining high coverage in sequence depth. See, Ng et al., Nature 461, 272-276, 2009 and Choi et al., PNAS USA 106, 19096-19101, 2009.

As used herein the term “whole transcriptome sequencing” refers to determining the expression of all RNA molecules including messenger RNA (mRNA), ribosomal RNA (rRNA), transfer RNA (tRNA), and non-coding RNA. Whole transcriptome sequencing can be done with a variety of platforms for example, the Genome Analyzer (Illumina, Inc., San Diego, Calif., USA) and the SOLID™ Sequencing System (Life Technologies, Carlsbad, Calif., USA). However, any platform useful for whole transcriptome sequencing may be used.

The term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq includes whole transcriptome sequencing as well as target specific sequencing.

The term “whole genome sequencing” or “WGS” refers to a laboratory process that determines the DNA sequence of each DNA strand in a sample. The resulting sequences may be referred to as “raw sequencing data” or “read.” As used herein, a read is a “mappable” read when the sequence has similarity to a region of a reference chromosomal DNA sequence. The term “mappable” may refer to areas that show similarity to and thus “mapped” to a reference sequence, for example, human genome (HG38) database.

In addition to “WGS,” the genomic compendiums may be obtained using targeted sequencing. In contrast to WGS, the term “targeted sequencing,” as used herein, refers to a laboratory process that determines the DNA sequence of chosen DNA loci or genes in a sample, for example sequencing a chosen group of cancer-related genes or markers (e.g., a target). In this context, the term “target sequence” herein refers to a selected target polynucleotide, e.g., a sequence present in a DNA molecule, whose presence, amount, and/or nucleotide sequence, or changes therein, are desired to be determined. Target sequences are interrogated for the presence or absence of a somatic mutation. The target polynucleotide can be a region of gene associated with a disease, e.g., cancer. In some embodiments, the region is an exon.

As used herein, the term “bin” refers to a group of DNA sequences grouped together, such as in a “genomic bin.” In a particular case, the bin may comprise a group of DNA sequences that are binned based on a “genomic bin window,” which includes grouping DNA sequences using genomic windows.

Methods and systems disclosed herein support large-scale, automated statistical analysis of proteomic variants, exomic variants, genetic variants and their associations with phenotypes (e.g., druggability or drug resistance), on a rolling basis, as genetic variant and phenotype data for new subjects, are added over time. For example, in some embodiments, the statistical association analysis that is performed is a genome-wide association study (GWAS) statistical analysis. In a GWAS analysis, one determines what genes or genetic variants are associated with a phenotype of interest. In some embodiments, the genetic variant data are obtained from genomic sequencing of the subject's sample containing nucleic acids. In another aspect, the genetic variant data are obtained from exome sequencing (e.g., whole exome) of the subject's sample containing nucleic acids. In another aspect, the genetic variant data are obtained from proteomic sequencing or even 3D structure modeling of a portion or the entirety of the subject's proteome.

The term “mapping” refers to a method for describing a position of a genetic locus in terms of recombination frequency with a genetic polymorphism. The results of a mapping method are described in map units.

As used herein, the term “screen” refers to a specific biological or biochemical assay which is directed to measurement of a specific condition or phenotype that a molecule induces in a target, e.g., target in silico system (e.g., computational modeling software based on energy considerations), target cell-free systems (e.g., BIACORE systems), target cells, tissues, organs, organ systems, or organisms.

As used herein, the term “selecting” in the context of screening compounds or libraries includes both (a) choosing compounds from a group previously unknown to be modulators of a condition or phenotype (e.g., cancer); and (b) testing compounds that are known to be inhibitors or activators of the condition or phenotype (e.g., cancer). Both types of compounds are generally referred to herein as “test compounds.” The test compounds may include, by way of example, polypeptides (e.g., small peptides, artificial or natural proteins, antibodies), polynucleotides (e.g., DNA or RNA), carbohydrates (small sugars, oligosaccharides, and complex sugars), lipids (e.g., fatty acids, glycerolipids, sphingolipids, etc.), mimetics and analogs thereof, and small organic molecules having a molecular weight of less than about 10 KDa, preferably less than about 5 KDa, especially less than about 1 KDa (e.g., about 300 daltons to about 800 daltons). The test compounds may be provided in library formats known in the art, e.g., in chemically synthesized libraries, recombinantly expressed libraries (e.g., phage display libraries), and in vitro translation-based libraries (e.g., ribosome display libraries).

As used herein, the term “tolerant” when used in reference to a molecule (e.g., a protein or a binding pocket therein), means that the particular molecule, shows less of an effect, or no effect, in response to a variation in its structure (e.g., primary, secondary, tertiary or even quartnery structure) as compared to a corresponding control (e.g., wild-type protein or a binding pocket therein).

Routine scoring methods may be used to delineate whether a protein or a binding pocket therein is tolerant or intolerant to a variation. It should be understood that protein tolerance to variation, although influenced by amino acid sequences, also depends on other physiochemical factors. Accordingly, tolerance is preferably expressed in relative terms (e.g., highly tolerant, relatively tolerant, neutral, relatively intolerant, or highly intolerant).

Outcomes of scoring methods can broadly be divided into absolute (e.g., rank) and relative (e.g., percentile) comparisons. Rank metrics may be further divided into relative ranks (e.g., bottom 20%, bottom 10%, or bottom 5%; top 40%, top 20% or top 10%) absolute ranks (e.g., top 5 out of 40,000 sites). Percentile or quantile statistics may be used to characterize a candidate's tolerance in reference to a population (e.g., comparing a subject protein in reference to a proteome or in reference to a population of structurally similar proteins, i.e., homologs).

### Nucleic Acid Sequences

Described herein, are methods, systems, and media useful for determining a 3DTS of one or more amino acids of a protein. The 3DTS score is calculated based upon the propensity to vary of nucleic acid sequence that encodes protein. Nucleic acid sequence variants that result in a missense mutation that vary more highly then the average mutation rate of the genome, protein or genomic locality are tolerant to variation. Nucleic acid sequence variants that result in a missense mutation that vary less than the average mutation rate of the genome, protein or genomic locality are intolerant to variation. In theory, a nucleic acid sequence variant that encodes a missense mutation that does not vary (e.g., a variant that is never observed) is perfectly intolerant to variation. The nucleic acid sequences that are used to determine a given variant's mutation rate can be any nucleic acid suitable for the purpose and includes DNA and RNA. DNA sequence data appropriate for the methods described herein will usually be generated from whole genome/exome sequencing, but can also be obtained from a targeted sequencing of multiple individuals or a database comprising DNA sequence data from many individuals. RNA sequence data appropriate for the methods described herein are generally reflected in cDNA sequencing of reverse transcribed RNA templates. In certain embodiments, the DNA sequence comprises a sequence for an individual's whole genome, or only the high confidence regions of an individual's whole genome. In certain embodiments, the DNA sequence comprises a sequence for the high confidence region of an individual's whole genome as defined by the NA12878 Genome-In-A-Bottle call set (GiaB v2.19). In certain embodiments, the DNA sequence comprises a sequence for 90% of the high confidence region of an individual's whole genome as defined by the GiaB v2.19. In certain embodiments, the DNA sequence comprises a sequence for 80% of the high confidence region of an individual's whole genome as defined by the GiaB v2.19. In certain embodiments, the DNA sequence comprises a sequence for 70% of the high confidence region of an individual's whole genome as defined by the GiaB v2.19. Nucleic acid variants can be determined by alignment to a reference genome, for example, the publicly available GRCh38 (hg38) released in December 2013. Alternatively, the methods can employ a reference genome determined from a plurality of genomes that is constructed ad hoc.

### Types of Mutations

The types of genomic variants and mutations that are useful for calculating the 3DTS are missense mutations. Missense mutations are those types of nucleic acid mutations that result in amino acid change. This is in contrast to synonymous mutations, which result in no underlying change in amino acid sequence.

### Global Mutation Rate

The global mutation rate is the background or constant mutation rate one would expect to see if any given variant leading to a missense mutation were selected randomly or occurred in the absence of any selection pressure. This can also be referred to as an expected mutation rate or variance rate. This can be estimated by looking at the mutation rates of variants expected to be under a low degree of selection, for example, synonymous variants or variants from non-coding regions (outside of splice junctions, promoter and enhancer sequences). In certain embodiments, the global mutation rate is the mutation rate defined by the background rate of mutation for non-coding bases. This global mutation rate can be determined by looking at the overall rate of mutation for synonymous or non-coding variants in a plurality of genomes for example greater than 1,000, 10,000, 50,000, 100,000 or more genomes, including increments therein. Another source for this rate would be from exome data derived from different individuals in some cases greater than 10,000, 50,000, 100,000 or more exomes, including increments therein, can be analyzed to arrive at a global mutation rate. The global mutation rate can be calculated from whole genome sequencing, exome sequencing, or SNP typing. In certain embodiments, a global mutation rate can be calculated regionally with respect to a given gene. For instance, the mutation rate can be calculated for all bases within 1 kilobase, 10 kilobases, 100 kilobases, 1 megabases, 5 megabases, 10 megabases, 50 megabases, or 100 megabases, including increments therein, of a gene for which a 3DTS score is being calculated. The estimated global mutation rate can be treated as a constant. In a certain aspect, the global mutation rate is between about 1×10⁻⁵ and about 1×10⁻⁷, between about 1×10⁻⁶ and about 1×10⁻⁷ between about 1×10⁻⁶ and about 5×10⁻⁶, between about 1×10⁻⁶ and about 5×10⁻⁶, between about 2×10⁻⁶ and about 4×10⁻⁶, or between about 2×10⁻⁶ and about 3×10⁻⁶. In a certain aspect, the global mutation rate is about 1×10⁻⁶, 2×10⁻⁶, 3×10⁻⁶, 4×10⁻⁶, 5×10⁻⁶, 6×10⁻⁶, 7×10⁻⁶, 8>10⁻⁶ or 9×10⁻⁶. In a particular aspect, the global mutation rate is about 2.5×10⁻⁶. The global mutation rate can also take into account the fact that some amino acid substitutions are conservative (e.g., a charged amino acid for an amino acid of the same charge), and may have a minor impact on protein structure or function. The global mutation rate can be the expected rate of variation for an entire genome, high-confidence areas of a genome, a specific protein or a specific range of nucleotides. For example, about 1,000, 5,000, 10,000, 100,000 or more nucleotides around a specific variant, including increments therein. The global mutation rate can be a rule of an algorithm that defines the background mutation rate and is approximated even though the “true background mutation rate” is unknown or incalculable.

### Variant Mutation Rate

The variant mutation rate is the mutation rate for any given variant leading to a missense mutation. As opposed to the global mutation rate, the variant mutation rate would be the rate actually observed at a particular locus. The variant mutation rate can be the rate observed in a plurality of sequences from a nucleotide dataset; for example, nucleotide variation data from greater than 1,000, 10,000, 50,000, 100,000, 500,000, or 1,000,000 different individuals, including increments therein, may be taken into account to establish a variant mutation rate. The variant mutation rate can also take into account the fact that a variant need not give rise to a missense mutation because of codon degeneracy. For example, a nucleic acid sequence can code for a residue that is highly intolerant to mutation, but a variant in that sequence that does not result in a nucleic acid change would have no impact on the variant mutation rate. In a certain embodiment the variant mutation rate only takes into account nonsynonymous variations at a given sequence locus. The variant mutation rate can be a rule of an algorithm that defines an observed mutation rate in a given data set and is approximated even though the “true variant mutation rate” is unknown or incalculable. The accuracy of this rule increases as more distinct nucleotide datasets are analyzed.

Mutations based on the intergenic rate genome-wide

In some embodiments, the methods and systems of the disclosure also take into consideration, intergenic rate of mutations, genome-wide. As is known in the art, intergenic regions (IGR) are stretches of DNA sequences located between genes, which primarily include noncoding DNA. Occasionally some intergenic DNA acts to control genes nearby (e.g., promoters, regulators, enhancers, repressors, etc.), but most of it has no currently known function. Experimental evidence indicates that about 98.5% of 3D sites do not contain a common missense variant (AF>0.05) and such sites would not be affected by the incorporation of an allele frequency term. Through incorporate of context (k-mer) expectation of variation, the algorithms and methods for determining DTS scores may be further refined.

A basic approach in including intergenic mutations in the model is as follows: begin with the assumption that mutations in these regions do not confer deleteriousness; encode constraints that involve quantification of the differences between the coding region of interest and the neutral territories. Herein, nucleotide context dependent estimates may be incorporated by partitioning all intergenic loci by the 7-mer (heptamer) which symmetrically spans the locus. Next, a maximum likelihood estimate specific for each heptamer is computed.

Mutations based on the intergenic rate specific to a chromosome.

In addition to the inclusion of information on genome-wide intergenic mutation rate, the systems and methods of the disclosure may also include such information in the context of the chromosome. Chromosome-specific intergenic mutation rates provide valuable cues for mapping a particular protein to particular chromosomes. Mutations are often associated with recombination and certain areas in certain chromosomes recombine more actively/frequently than other chromosomes. Chromosomes with more hotspots would likely have higher mutation rates than other chromosomes, on average. In addition, purely for statistical reasons, larger chromosomes are more susceptible to mutation since they have a larger area in which they can accumulate damage. Further, research shows that regions located in the middle of a chromosome are less likely to contribute to genetic variation of traits than those at the ends. In other words, a gene's location on a chromosome influences the range of physical differences among different traits.

Information on chromosome-specific intergenic mutation rates may be included in the systems and methods of the present disclosure as described previously.

### Nucleotide Sata Sets

Nucleotide datasets for use in determining either the variant specific mutation rate or the global mutation rate comprise any suitable dataset with genomic data from a plurality of individuals. These can comprise SNP data, whole genome sequencing data, exome-sequencing data, or targeted resequencing data from a plurality of individuals. The datasets can be publicly available or private and comprise only variants in .txt, or .vcf format. In some cases, the quality of determining the variant specific mutation rate or the global mutation rate increases with increasing the amount of individuals represented by the dataset. In certain embodiments, the nucleotide data set can represent greater than 1,000, 10,000, 50,000, 100,000, 200,000, 500,000, 1,000,000 or more individuals, including increments therein. In certain embodiments, the dataset comprises data representative of different ethnicities, nationalities, or geographic regions. Mutation intolerance

Mutation intolerance represents a relative quantification of the tolerance of a given amino acid or functional domain of a protein to change. In other words, mutation intolerance represents a departure from the global mutation rate for a given missense-creating variant. An amino acid residue or functional feature of a protein is mutation intolerant if a given variant (or set of variants) occurs (or is observed in a plurality of individuals) at a rate, that is less than the global rate (e.g., the expected rate). These can be scored for example on a scale from 0 to 1, with 0 signifying that no missense variants are found at a given position (highest degree of intolerance), and 1 reflecting that missense mutations are found at a rate at or near the rate that would be expected for a variant under no selection pressure (highest degree of tolerance). This intolerance can be expressed and analyzed in many ways for example by ranking, creating a ratio, or a mathematical function that allows comparison of an expected rate and an observed rate across different variants. In a certain embodiment, the residue is defined as tolerant or intolerant. In a certain embodiment, the amount of intolerance is quantified so that different residues or features of a protein can be compared. In a certain embodiment, a threshold for intolerance is established for residues that have a mutation rate at least 20%, 30%, 40%, 50%, 60%, 70%, 80%, 90%, 100%, 200%, 300%, 400%, 500%, or 600%, lower than the expected rate, including increments therein. In a certain embodiment, a threshold for intolerance is established for residues that have a mutation rate at least 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold lower than the expected rate, including increments therein. In a certain, embodiment, mutation intolerance can be normalized or averaged across a plurality of amino acids, such as 2, 3, 4, 5, 6, 7, 8, 9, 10, 11, 12, 13, 14, 15, 16, 17, 18, 19, 20 or more amino acids, or by domain or feature.

Residues can also be defined as intolerant by spatial proximity (as opposed to covalently bonded) to a highly intolerant residue or plurality of residues. An initial set of protein tolerance rankings or scores can be further refined using structural data (e.g., X-ray crystallography, NMR, or cryoelectron microscopy). For example, a residue that is not immediately connected to an intolerant residue by a peptide bond can be defined as intolerant due to its spatial position within 2, 3, 4, 5, 6, 7, 8, 9, 10 or more angstroms of another intolerant residue.

Protein Domains and Features

Mutation intolerance can be defined for a single given amino acid or a plurality of amino acids, such as 2, 3, 4, 5, 6, 7, 8, 9, 10, 11, 12, 13, 14, 15, 16, 17, 18, 19, 20, 25, 30, 35, 40, 45, 50 or more amino acids, including increments therein. In a certain embodiment, the degree of mutation intolerance is established for a given protein domain or feature, including functional and structural features. In a certain embodiment, any feature which can be defined by structural motifs (e.g., beta sheets, alpha helices, coiled coil domains), sequence motifs (e.g., glycosylation, lipidation or phosphorylation sites), protein family relationships (e.g., conserved protein-protein interaction domains, IgG-like domains), or topologically (e.g., transmembrane, intracellular, or extracellular domains). In certain embodiments, the protein feature is selected from the list consisting of: an active site, a metal binding site, a chemical binding site, a DNA binding site, a nucleotide binding site, a zinc finger, a calcium binding site, a transmembrane domain, an intra membrane domain, a lipidation site, a glycosylation site, a phosphorylation site, a coiled-coil, an alpha helix, and a beta strand.

### Mapping Mutation Intolerance

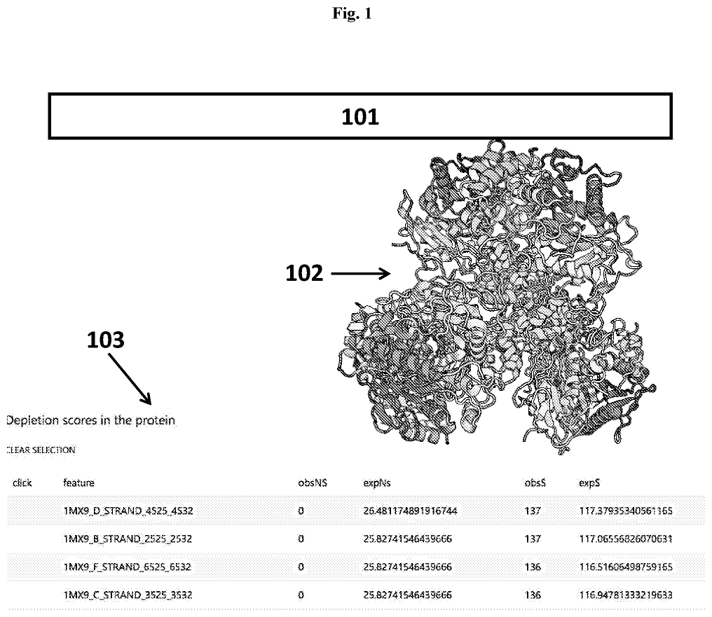

It is further contemplated that mutation intolerance can be mapped onto a representation of a protein; this representation can comprise a primary sequence or a three-dimensional structure. The three-dimensional structure can comprise any suitable means of displaying a protein, and includes ribbon diagrams and space filling models. The representation can be derived from publicly available databases that contain structural data. Referring to FIG. 1 a suitable used interface can comprise a search box 101 for entering search terms such as desired proteins, classes of proteins, or keywords associated with (a) given protein(s). A visual representation of the protein can be shown 102, this visual representation can be rotatable around an x, y, or z axis, flappable over an axis, or zoomable down to the individual residue level. Also shown can be an interactive table for data 103 showing data such as individual intolerance scores, which are sortable or filterable, downloadable, shareable, or exportable.

Workflow

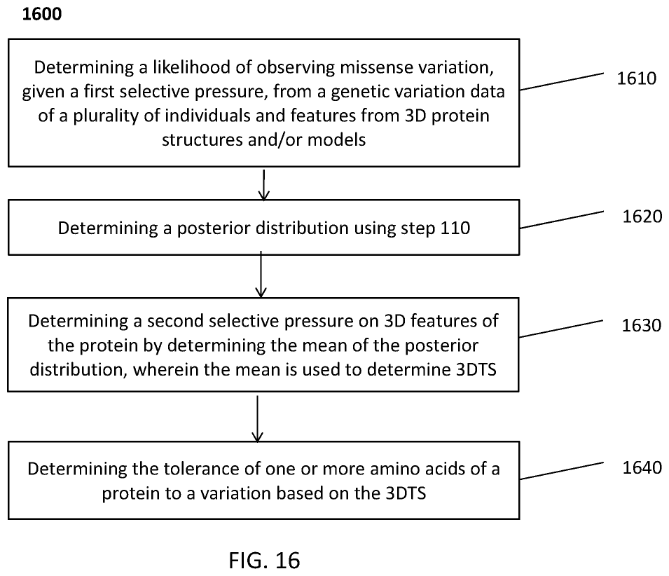

FIG. 16 shows a schematic diagram of the workflow of the disclosure and is used to identify whether a protein (or a binding pocket therein) is tolerant or intolerant to variation. There are many potential downstream applications to this technology, e.g., screening for druggable targets; identifying drug insensitive or resistant variants; identifying additional sites for target modulation (e.g., to elicit additive or even synergistic effects on a target); fine-tune target modulation (e.g., using a plurality of active and/or allosteric modulators); comparatively assess the properties of a plurality of modulators that share a common mode of operation (e.g., β lactam antibiotics).

In step 1610 of method 1600 of FIG. 16, a likelihood of observing missense variation, given a first selective pressure, from a genetic variation data of a plurality of individuals and features from 3D protein structures and/or models is determined. This step takes into consideration missense mutations that may arise, e.g., through evolutionary pressure or conservation pressure. Generally, the probability of observing at least one missense mutation in R samples in a single locus is l−exp(−pₗ*sₗ*bₗ*Rₗ, and this is computed in this step.

In step 1620 of method 1600 of FIG. 16, a posterior distribution on the selective pressure is determined using aforementioned step 1610. The posterior distribution may be defined using Bayes theorem, which combines a prior distribution and likelihood function. Generally, the prior function can be expressed as P(s)=U(0,1) and the likelihood function can be expressed as L(k s)=Poi(k, Σₗ^{K} l−exp(−pₗ*s*bₗ*Rₗ)). By numerical integration (Gauss-Legendre quadrature and importance sampling), the posterior mean on s with a uniform U(0,1) prior can be calculated.

In step 1630 of method 1600 of FIG. 16, a second selective pressure on 3D features of the protein is determined by evaluating the posterior distribution, wherein the mean is used to determine a three-dimensional tolerance score (3DTS). Generally, the the mean of the posterior is provided by E[P(s|k)]=∫₀¹sP(s|k)ds. The mean of the posterior distribution is used to calculate the 3DTS score, which may be expressed in rank basis or percentile basis, as provided above.

In step 1640 of method 1600 of FIG. 16, the tolerance of one or more amino acids of a protein to a variation is determined from the 3DTS score. If the 3DTS is below a threshold (e.g., below 10^{th}, 20^{th}, 30^{th} or 40^{th} percentile; preferably below 20^{th} percentile) then the amino acid is defined as being intolerant to the variation; if the 3DTS is above a threshold (e.g., above 50^{th} or 60^{th} percentile; preferably above 60^{th} percentile) then the amino acid is defined as being tolerant to the variation.

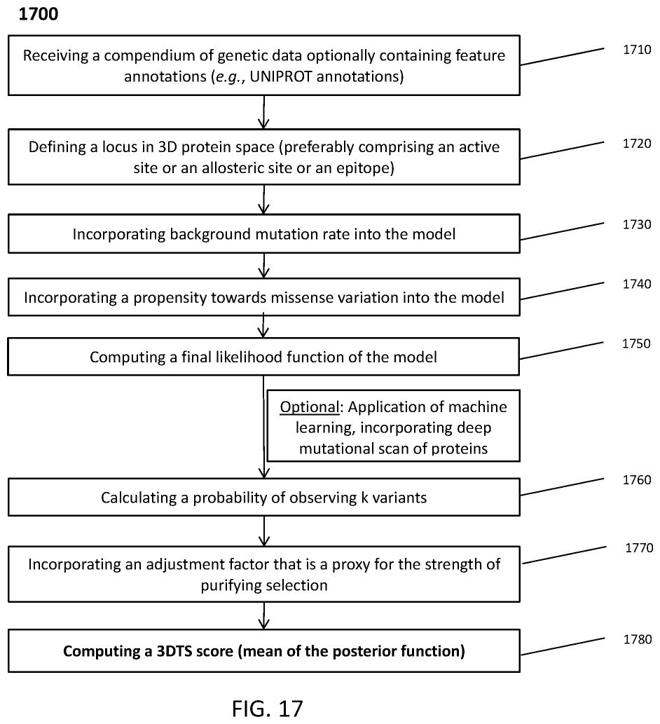

FIG. 17 shows a flow chart illustrating an embodiment method 1700 for identifying whether a protein (or a binding pocket therein) is tolerant or intolerant to variation. Method 1700 is illustrative only and embodiments can use variations of method 1700. Method 1700 can include steps for receiving sequence data and feature data from a plurality of individuals (e.g., genetic or exomic sequence data in FASTA/WIG/BED format; proteomic data in flat text, XML, RDF/XML, format optionally containing UNIPROT annotations of features); defining a locus in 3D protein space; and calculating a 3D Tolerance Score (3DTS) based on the a background mutation rate (parameter p); a propensity towards missense variation (Parameter b) and an adjustment factor that is a proxy for the strength of purifying selection (Parameter s).

In step 1710 of method 1700 of FIG. 17, a compendium of genetic data is received. Any form of genetic data, e.g., mRNA or cDNA sequence, gDNA sequence or proteomic sequence, may be received. In some embodiments, a set of 100,000+ exomes and 15000+ whole human genomes from gnomAD is received in a file. Additionally, a feature annotation may be included. Feature annotations may be obtained from Uniprot text files and cross-referenced from Gencode. Features include secondary structure elements (helix (HELIX), beta strand (STRAND), turn (TURN)) and others: binding site (BINDING), modified residue (MOD_RES), mutagenesis (MUTAGEN), region (REGION), motif (MOTIF), nucleotide binding (NP_BIND), natural variant (VAR_SEQ), active site (ACT_SITE), metal binding (METAL), disulfide bond (DISULFID), glycosylation (CARBOHYD), site (SITE), peptide (PEPTIDE), domain (DOMAIN), DNA binding (DNA_BIND), repeat (REPEAT), signal (SIGNAL), cross-link (CROSSLNK), lipidation (LIPID), propeptide (PROPEP), calcium binding (CA_BIND), topological domain (TOPO_DOM), zinc finger (ZN_FING), coiled-coil (COILED), compositional bias (COMPBIAS), transmembrane (TRANSMEM), intramembrane (INTRAMEM), transit peptide (TRANSIT), and non-standard residue (NON_STD).

In step 1720 of method 1700 of FIG. 17, a locus in 3D protein space is defined (preferably comprising an active site, an allosteric site, or an epitope). A radius around a protein feature that may be 1, 2, 3, 4, 5, 6, 7, 8, 9, 10 or more Angstroms defines a 3D site. The corresponding nucleotides (loci) defined by the 3D site are evaluated in this model.

In step 1730 of method 1700 of FIG. 17, background mutation rate is incorporated into the model. Generally, this is the expected number of mutations at a locus assuming all mutations at a locus are neutral. This is done by fitting the observed number of synonymous variants across all proteins to the expected number of synonymous variants by fixing s=1. A synonymous local mutation rate can also be used, which estimates heterogeneity across the genome, and is calculated as above, but is only evaluated on a single protein chain. In addition to these two methods of estimating a background/neutral mutation rate, a genome-wide intergenic variation rate or a chromosome-specific intergenic variation rate can be estimated from non-coding variation (further maximizing the likelihood function, computed in step 1750). Additionally, a nucleotide-context dependent estimate (heptamer symmetrically spanning the reference nucleotides) can be used to estimate mutation rate heterogeneity.

In step 1740 of method 1700 of FIG. 17, a propensity towards missense variation is incorporated into the model. Each reference nucleotide in a 3D-defined locus has 0, 1, 2, or 3 chances of a single nucleotide variation leading to a missense variant, defined as parameter b. This is determined based on the protein isoform for the 3D structure, the transcript encoding this protein isoform and the reference genome encoding this transcript for the locus. This parameter is normalized to 1.

In step 1750 of method 1700 of FIG. 17, a final likelihood function of the model is computed. Here, the probability of observing a missense mutation at a locus l is defined by the background mutation rate (p), the propensity towards missense variation (b), and an adjustment factor that serves as a proxy for the strength of purifying selection (s): pₗ*sₗ*bₗ. Herein, the sequencing data for each person (i.e., each sample) is treated as a separate Bernoulli trial (i.e., presence or absence of a variant resulting in a missense mutation). At a given locus, all parameters are the same across the samples, thus aggregating R samples yields a binomial distribution as the number of samples with a missense mutation at locus l. Using the Poisson approximation, the probability of observing at least one missense mutation in R samples in a single locus is l−exp(−pₗ* sₗ*bₗ* Rₗ). Since each locus has different bₗ and Rₗ parameters, this is considered when aggregating over K loci (i.e., aggregating over the 3D feature). Thus, aggregating over these K>1 loci into a single value is the sum of Bernoulli trials of heterogeneous parameters, which may be approximated using a Poisson distribution following Le Cam's theorem. Thus, the final likelihood function of the model is: P (observed k variants in K loci among R samples|pₗ, s, bₗ)=Poi(k,Σₗ^{K} l−exp(−pₗ s bₗ Rₗ)). Note: the bₗ parameter is a function of the genetic code, while the pₗ parameter is learned. Neutral sections of the genome are used to estimate this pₗ mutation rate parameter by setting s equal to 1 (assuming these sections of the genome are not deleterious) and the likelihood function is maximized under these constraints.

In step 1760 of method 1700 of FIG. 17, the probability of observing k variants is calculated. This may be accomplished using Gauss-Legendre quadrature or via importance sampling. The probability function is represented by the equation P(k)=∫₀¹L(k|s)*P(s)ds.

In step 1770 of method 1700 of FIG. 17, an adjustment factor that is a proxy for the strength of purifying selection is incorporated into the model (s). Herein, a value of s=1 means the variants are as expected based on the background mutation rate (i.e., neutral effect) while a value of s=0 means the locus is completely depleted of variation (i.e., intolerant). This may be represented in Bayesian format as

$$P ( s \vert k ) = \frac{L ( k \vert s ) * P ( s )}{P ( k )} .$$

In step 1780 of method 1700 of FIG. 17, a 3DTS score is computed. This is the mean of the posterior of the probability function computed in step 170 and is defined as E[P(s k)]=∫₀¹sP(s|k)ds.

Generally in method 1700 of FIG. 17, a machine learning approach may be incorporated to systemically determine, for example, the background mutation rate, the propensity towards missense variation, or generally determine the likelihood function. The approach may be applied at any step of the method, although it may be advantageous to implement the machine learning at step 1750. In this regard, in the purely illustrative method of FIG. 17, a machine learning (ML) algorithm is optionally applied at step 1750 to build the model. The ML algorithm may comprise employing a deep learning algorithm such as, e.g., using neural networks, with applicable training data sets and specific weighthing factors optimized by backpropogation, to analyze variations in protein motifs, domains, epitopes or binding sites and deduce the functional significance thereof.

In some embodiments, the ML is trained with an in silico dataset. For example, the in silico dataset may include deep mutational scanning of proteins. For example, as described in detail in Examples section, deep mutational data is available for PPARG, wherein every amino acid residue in the primary structure of the protein has been mutated and the functional significance of each amino acid elucidated via a functional assay (see, Majithia et al., Nat Genet., 48(12): 1570-1575, 2016). The dataset may include deep mutational scanning of other proteins, e.g., MAPK1/ERK2, p53, PTEN, TPMT, UBE2I , SUMO1, TPK1, CALM1, CALM2, CALM3, BRCA1 or YAP65 or a domain therein, where available. Similarly, functional assays can be designed to examine the effects of deleterious missense mutations as opposed to benign mutations for other target proteins and datasets containing functional annotations for each and every amino acid of the proteins be constructed as in the case of PPARG, above. Next, 3DTS scores of the individual amino acids in the protein targets (e.g., PPARG) is compared to the deep mutation data on the functional significance thereof, as determined by single-variant assays. Optionally, data from 3D modeling software such as CADD is integrated into the comparative model. Being able to combine 3DTS with existing scores (e.g., CADD) improves the predictive power and also the accuracy of the model, with respect to identifying intolerant sites with confidence. Furthermore, through the robust integration of ML, the final likelihood function of the model may be further refined across a wide spectrum of target molecules.

The architecture of the machine learning approach will be discussed in greater detail below.

Machine Learning (ML)

Not being bound to a single embodiment and purely for the purpose of illustration, a machine-learning algorithm was integrated into the existing methodology at an individual, or combination of individual steps, in accordance with various embodiments herein. ML can be incorporated to optimize the results coming out of the algorithm (e.g., neural network, ML algorithm, etc.), by utilization of inputted training data sets, cross reference of output to known answers, backpropagation, and adjustment of weighting factors and parameters associated with the given ML algorithm in a repeating loop to arrive at a threshold quality of data output. For instance, neutral sections of the genome were used to estimate this pₗ mutation rate parameter by setting s equal to 1 (assuming these sections of the genome are not deleterious) and the likelihood function is refined (e.g., optimized or trained) under these constraints. In subsequent steps, the prediction power of the model on the test dataset may be validated, e.g., using a probability model such as logistic regression (e.g., optimized or trained in conjuction or in the alternative). Optionally, a resampling may be performed to obtain an unbiased appraisal of the model's likely future performance. Features of ROC curve, such as, area-under-the curve (also called c-index) or concordance probability from a statistical test such as the Wilcoxon-Mann-Whitney test, may provide a good summary measure of pure predictive discrimination.

Modulators of Intolerant Regions

The method of determining intolerance and three-dimensional tolerance scores described herein are particularly useful for research in drug design and development. Protein domains, features or regions that are intolerant to mutation provide for potential drug targets. In certain embodiments, any of the domains, features, regions, or amino acids that rank within the top 1%, 5%, 10%, or 20% of intolerance by 3DTS, including increments therein can be a potential drug target. In certain embodiments, that drug is an inhibitor or antagonist of the particular protein; in other embodiments, the drug is an activator or agonist of the particular protein. These types of antagonists or antagonists are useful for therapeutic intervention. In certain embodiments, that drug is an antibody or antigen binding fragment thereof that acts as an antagonist or an agonist. In certain embodiments, the antagonist or agonist acts at a site that is not the active site for the protein, not a protein-protein interaction site, or a protein-nucleic acid binding site.

Methods of Screening

In some embodiments, the disclosure relate to systems and methods for screening compounds that bind to and/or modulate (e.g., inhibit) a target of interest, e.g., a target selected from MAPK1/ERK2, p53, PTEN, TPMT, UBE2I , SUMO1, TPK1, CALM1, CALM2, CALM3, BRCA1 or YAP65 or a domain therein. Preferably, the screened compounds interact with and/or bind to binding pockets of the proteins or epitopes of the antigens. Non-covalent molecular interactions important in this association include hydrogen bonding, van der Waals interactions, hydrophobic interactions and electrostatic interactions.

Second, the interacting compound is able to assume a conformation that allows it to associate with the binding pocket (e.g., located in an active site or an allosteric site or an epitope) directly. Although certain portions of the compound will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the binding pocket.

The potential inhibitory or binding effect of molecule on a binding pocket may be analyzed prior to its actual synthesis and testing by the use of computer modeling techniques. If the theoretical structure of the given entity suggests insufficient interaction and association between it and the binding pocket, testing of the entity is obviated. However, if computer modeling indicates a strong interaction, the molecule may then be synthesized and tested for its ability to bind to a binding pocket. This may be achieved by testing the ability of the molecule to modulate the target using assays described in the art. Thus, synthesis of inoperative compounds may be avoided.

A potential inhibitor of a binding pocket may be computationally evaluated by a series of steps in which chemical entities or fragments are screened and selected for their ability to associate with the binding pockets.

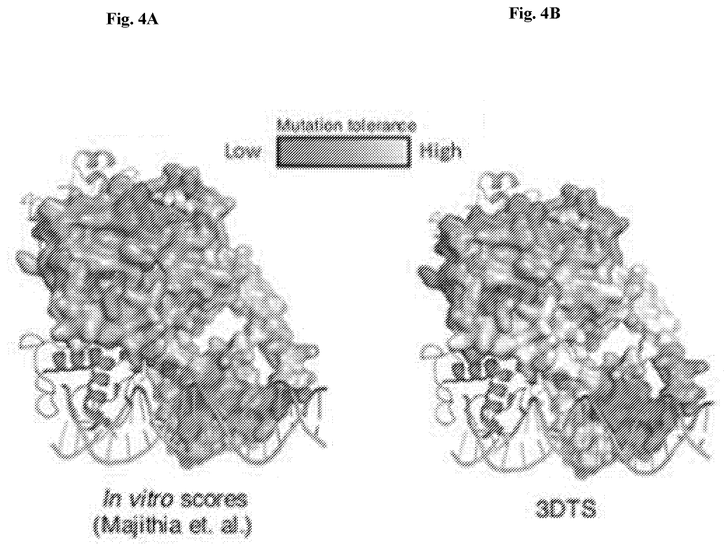

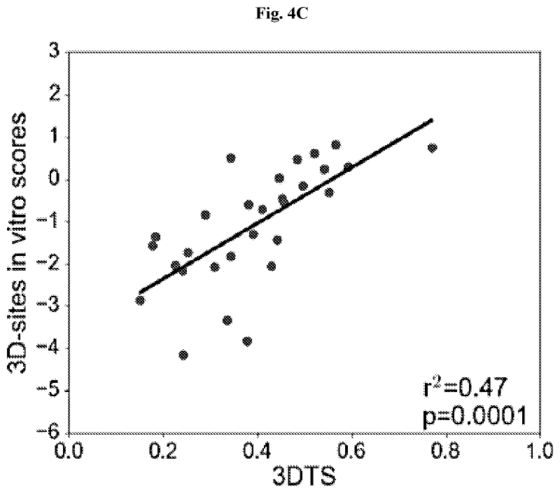

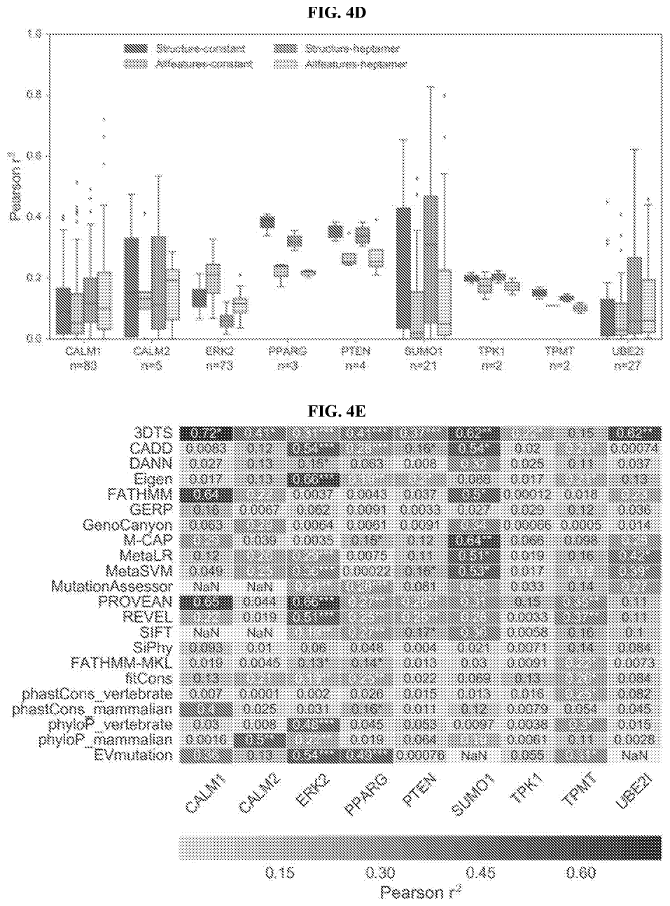

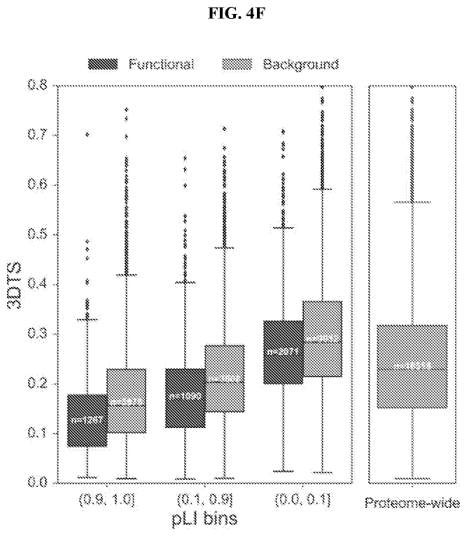

One skilled in the art may use one of several methods to screen chemical entities or fragments for their ability to associate with a binding pocket. This process may begin by visual inspection of, for example, a binding pocket on the computer screen based on the structure coordinates of the target (e.g., FIG. 4A or 4B) or other coordinates, which define a similar shape, generated from the machine-readable storage medium. Selected fragments or chemical entities may then be positioned in a variety of orientations, or docked, within that binding pocket as defined supra. Docking may be accomplished using software such as CADD™ and PYMOL™, followed by energy minimization and molecular dynamics with standard molecular mechanics force fields.

Specialized computer programs may also assist in the process of selecting fragments or chemical entities. These include: GRID, MCSS, AUTODOCK, DOCK, ALCHEMY™, LABVISION™, SYBYL™, MOLCADD™, LEAPFROG™, MATCHMAKER™, GENEFOLD™ and SITEL™, QUANTA™, CERIUS2™ X-PLOR, CNS, CATALYST, MODELLER™, CHEMX™, LUDI™, INSIGHT™, DISCOVER™, CAMELEON™ and IDITISm; RASMOL™; MOE™; MAESTRO; CHIME; MOIL; MACROMODEL™ and GRASP™; RIBBON; NAOMI; EXPLORER EYECHEM™; UNIVISION™; MOLSCRIPT™; CHEM 3D™ and PROTEIN EXPERT™; CHAIN; SPARTAN, MACSPARTAN and TITANS; VMD™; SCULPT™; PROCHECK™; DGEOM; REVIEW; HYPERCHEM™; PKB; GROWMOL; MICE; MCPro; CAVEAT™; and 3D database systems such as ISIS™.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target. This would be followed by manual model building using software such as CADD™, PYMOL™, QUANTA or SYBYL™.

Instead of proceeding to build an inhibitor of binding pocket in a step-wise fashion one fragment or chemical entity at a time as described above, inhibitory or other binding compounds may be designed as a whole or “de novo” using either an empty binding site or optionally including some portion(s) of a known inhibitor(s) or activator(s).

Once a compound has been designed or selected by the above methods, the efficiency with which that entity may bind to the binding pocket may be tested and optimized by computational evaluation. For example, an effective binding pocket inhibitor must preferably demonstrate a relatively small difference in energy between its bound and free states (i.e., a small deformation energy of binding). Thus, the most efficient binding pocket inhibitors should preferably be designed with a deformation energy of binding of not greater than a threshold value, e.g., about 10 kcal/mol or even 1 kcal/mol. Binding pocket inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of software designed for such uses include, e.g., AMBER, QUANTA, and AMSOL. These programs may be implemented, for instance, using a Silicon Graphics workstation such as an INDIGO with IMPACT graphics. Other hardware systems and software packages will be known to those skilled in the art.

Another approach enabled by this disclosure is the computational screening of small molecule databases for chemical entities or compounds that can bind in whole, or in part, to a human a binding pocket. In this screening, the quality of fit of such entities to the binding site may be judged either by shape complementarity or by estimated interaction energy.

Preferably, the binding domain comprises a ligand binding domain or an allosteric domain of the following proteins MAPK1/ERK2, p53, PTEN, TPMT, UBE2I , SUMO1, TPK1, CALM1, CALM2, CALM3, BRCA1 (preferably the RING domain) and YAP65 (preferably the WW domain).

The screening methods of the disclosure are particularly useful in the context of identifying sites or motifs within a target protein (e.g., an enzyme or an antigen) that are intolerant to binding to a binding partner (e.g., an antagonist or an antibody), which permits screening binding partners that serve as drug candidates against the proteins. However, a similar methodology can be implemented to identify lack of druggability of targets (e.g., mutant proteins that differ from the wild-type sequence by one or amino acids, which render them undruggable with the same drug candidates that are effective against the wild-type counterpart). In the latter situation, the screening methodology can save valuable time and cost in the drug screening process and perhaps provide alternative avenues for targeted therapy, e.g., using genetic approaches such as RNAi or siRNA.

In some embodiments, the methods of screening drug candidates may be validated using downstream methods. For instance, functional interpretation of variant targets may involve construction of a cDNA library consisting of all possible amino acid substitutions in the protein target. The library is then introduced into target cells (e.g., in the context of PPARG, human macrophages edited to lack the endogenous PPARG) and stimulated with agonists to trigger functional activity (e.g., expression of CD36, a canonical target of PPARG). The cells are sorted (e.g., using FACS antibodies that can separate CD36+ and CD36− cell populations) and the transcriptomes are sequenced to determine the distribution of each variant in relation to the functional activity assayed for (e.g., CD36+ activity).

Use of systems/methods of the disclosure in drug therapy and identification of responders:

The methods of the disclosure allow for the identification of subjects in whom the composition for treating a disease is effective (i.e., patient responds to the therapeutic agent). For example, the identification of whether a subject has a variant protein permits assessment of whether the subject will respond to a standard treatment or not. Such assessments may be used, for example, in targeted therapy of diseases (e.g., cancer). For instance, based on the results of the aforementioned tests, certain types of drugs may be favored over other types of drugs in certain subjects based on whether the subject has a variant allele for the protein target, wherein the variant allele encodes a variant gene product having variations in the binding pocket (e.g., active site, an allosteric site or an epitope) to which a candidate drug binds. Depending on the change in the 3DTS score of the binding pocket as a result of the variations, the subject can be phenotypically identified (e.g., as drug sensitive or drug insensitive, e.g., herceptin sensitive or insensitive).

The disclosure provides methods for prognosticating the response of a patient to a composition that is useful for treating a disease, e.g., cancer. The predictive method comprises analyzing a biological sample obtained from a subject having the disease (e.g., cancer), which subject is currently or previously being treated with a composition (e.g., anticancer drug), wherein the biological sample contains genetic data on the target (e.g., protein target); determining the druggability or insensitivity of the target to the composition, wherein if the target is deemed druggable, then the subject is prognosticated as a likely responder to a therapy with the composition. Preferably, the prognostic method (i.e., measuring likelihood of response) is carried out by measuring 3DTS of the target protein, wherein, if the 3DTS is below a threshold value (e.g., 20^{th} percentile), then the subject is prognosticated as likely responding to the therapy with the composition. Conversely, if the 3DTS is above a threshold value (e.g., 50^{th} percentile), then the subject is prognosticated as likely not responding to the therapy with the composition.

The aforementioned identification and/or prognostic methods can also be used to monitor whether or not a patient is responding to an agent (e.g., an anticancer agent such as herceptin). As is known in the field of tumor biology, the rapid rate of mutations in the tumor tissue gives rise to variant drug targets that respond differentially to therapeutic drugs. Some variations give rise to chemo-therapy or immune-therapy resistant drug targets or cancers (e.g., Her2 negative breast cancer). Thus, the aforementioned methods can be used to identify whether the subject's genome or exome has undergone mutations such that the gene products of such mutations give rise to protein targets that have different 3DTS profiles (compared to other cancer patients or wild-type). By effective patient monitoring, revisions can be made on the course of therapy (e.g., switch from Herceptin to hormone therapy or therapy with bevacizumab in combination with chemotherapy in Her2 mutant patients that have altered 3DTS scores at pockets that bind to herceptin).

As with the other methods described herein, in the aforementioned methods for determining the subject's responsiveness to a test agent or clinically-approved therapeutic agent for treating diseases, e.g., cancer, may be carried out using various techniques, including simple comparisons, one or more statistical analyses, including combinations thereof.

The aforementioned methods are also useful in identifying responders and/or non-responders to novel therapeutic agents that may be at various stages of clinical testing. In particular, the aforementioned methods allow clinicians to stratify high-risk chemo-resistant or immunotherapy resistant individuals and to assess the efficacy of therapeutic candidates more effectively and safely. The methods of the disclosure not only provide cost-saving measures to pharmaceutical companies but also enable hospitals and dispensaries to deliver individualized and targeted therapy to patients by improving drug efficacy and reducing their side effects.

Digital Processing Device

The 3DTS can be calculated and communicated to users via various platforms, systems, media, and include a digital processing device, or use of the same. In further embodiments, the digital processing device includes one or more hardware central processing units (CPUs) or general-purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud-computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, and notebook computers.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft Windows®, Apple® Mac OS X®, UNIX®, and UNIX-like operating systems such as GNU/Linux®. In some embodiments, the operating system is provided by cloud computing.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

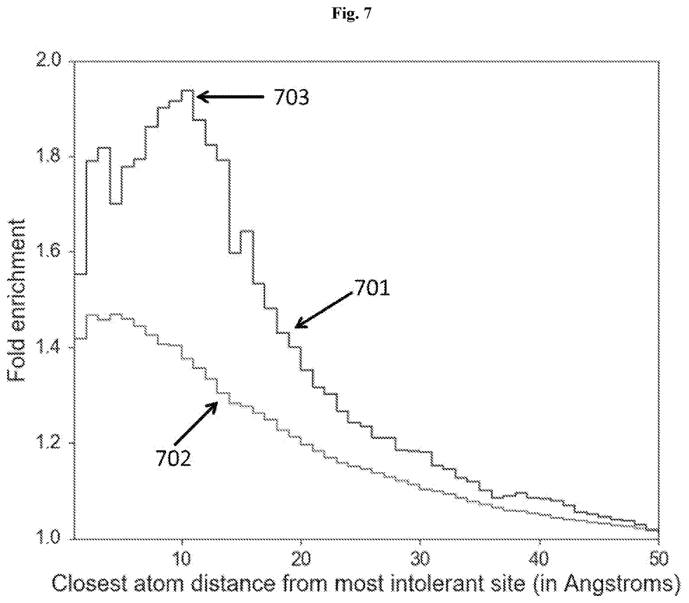

Referring to FIG. 7, in a particular embodiment, an exemplary digital processing device 1301 is programmed or otherwise configured to determine a three-dimensional tolerance score. The device 1301 can regulate various aspects of the methods and system of the present disclosure, such as, for example, determining global mutation rates, variant specific mutation rates, determining missense variants, determining intolerant amino acid residues, features, regions, and domains. In this embodiment, the digital processing device 1301 includes a central processing unit (CPU, also “processor” and “computer processor” herein) 1305, which can be a single core or multi core processor, or a plurality of processors for parallel processing. The digital processing device 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The digital processing device 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the device 1301, can implement a peer-to-peer network, which may enable devices coupled to the device 1301 to behave as a client or a server.

Continuing to refer to FIG. 7, the CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and write back. The CPU 1305 can be part of a circuit, such as an integrated circuit. One or more other components of the device 1301 can be included in the circuit. In some cases, the circuit is an application specific integrated circuit (ASIC) or a field programmable gate array (FPGA).

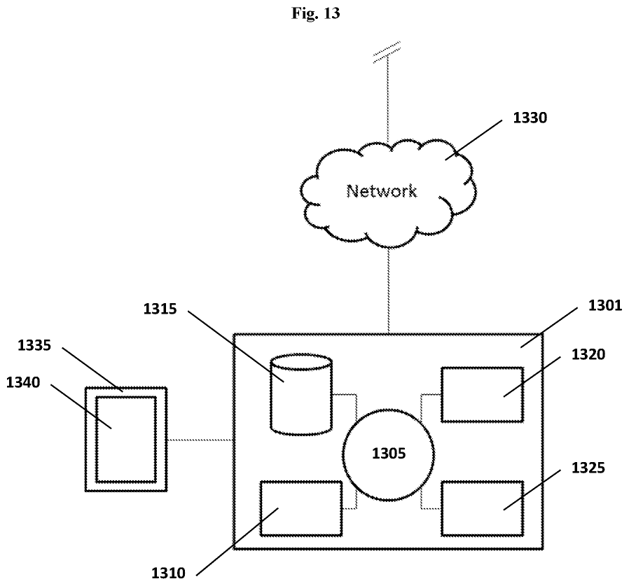

Continuing to refer to FIG. 13, the storage unit 1315 can store files, such as drivers, libraries and saved programs. The storage unit 1315 can store user data, e.g., user preferences and user programs. The digital processing device 1301 in some cases can include one or more additional data storage units that are external, such as located on a remote server that is in communication through an intranet or the Internet.

Continuing to refer to FIG. 7, the digital processing device 1301 can communicate with one or more remote computer systems through the network 1330. For instance, the device 1301 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 1301, such as, for example, on the memory 1310 or electronic storage unit 1315. The machine executable or machine-readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. The program and instructions may be permanently, substantially permanently, semi-permanently, or non-transitorily encoded on the media.

The disclosure relates to systems for determining a tolerance or intolerance of one or more amino acids of a protein to a variation, comprising, a module for determining a likelihood of observing missense variation, given a selective pressure, from a genetic variation data of a plurality of individuals and features from 3D protein structures and/or models; a module for determining a posterior distribution using a likelihood function and assuming a uniform prior on the selective pressure; and a module for determining a selective pressure on 3D features of the protein by determining the mean of the posterior distribution, wherein the mean is used to determine a three-dimensional tolerance score (3DTS) and wherein the 3DTS is indicative of the tolerance or intolerance of one or more amino acids of a protein to a variation.

The disclosure relates to systems for determining druggability of a protein, comprising, a module for determining a likelihood of observing missense variation, given a selective pressure, from a genetic variation data of a plurality of individuals and features from 3D protein structures and/or models; a module for determining a posterior distribution using a likelihood function and assuming a uniform prior on the selective pressure; and a module for determining a selective pressure on 3D features of the protein by determining the mean of the posterior distribution, wherein the mean is used to determine a three-dimensional tolerance score (3DTS) and wherein the 3DTS is indicative of the druggability of the protein.

The disclosure relates to systems for determining drug resistance potential of a variant protein, comprising, a module for determining a likelihood of observing missense variation, given a selective pressure, from a genetic variation data of a plurality of individuals and features from 3D protein structures and/or models; a module for determining a posterior distribution using a likelihood function and assuming a uniform prior on the selective pressure; and a module for determining a selective pressure on 3D features of the variant protein by determining the mean of the posterior distribution, wherein the mean is used to determine a three-dimensional tolerance score (3DTS) and wherein the 3DTS is indicative of the drug resistance potential of the variant protein.

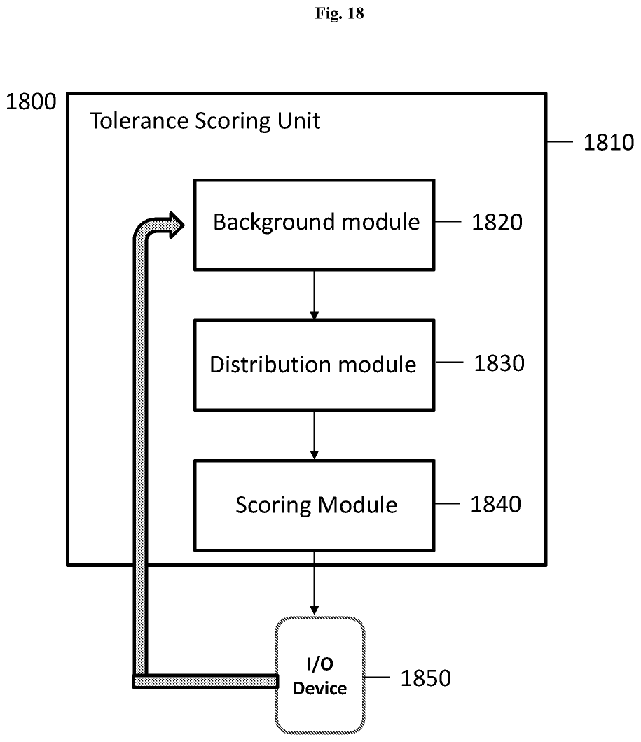

FIG. 18 shows a schematic diagram of a representative system 1800 of the disclosure. Specifically, a representative Tolerance Scoring Unit 1810 is shown, which is useful for determining a tolerance or intolerance of one or more amino acids of a protein to a variation. Tolerance Scoring Unit 1810 comprises three modules and can be communicatively connected to an input/output device (I/O device). A first module, Background module 1820 contains components and/or software for determining a likelihood of observing missense variation, given a first selective pressure, from a genetic variation data of a plurality of individuals and features from 3D protein structures and/or models. The Background module 1820 may be equipped to receive genetic, exomic or proteomic data comprising sequence of a protein of interest (or a binding pocket therein). The Background module 1820 is communicatively connected to a second module, the Distribution module 1830. Distribution module 1830 contains components and/or software for determining a posterior distribution using an output of the Background module 1820. Distribution module 1830 may determine the posterior distribution using a likelihood function and assuming a uniform prior on the selective pressure. The Distribution module 1830 is further communicatively connected to a third module, the Scoring module 1840. Scoring module 1840 contains components and/or software for determining a second selective pressure on 3D features of the protein by determining the mean of the posterior distribution, wherein the mean is used to calculate a three-dimensional tolerance score (3DTS). Scoring module 1840 is communicatively connected to an input/output (I/O) device, e.g., a server or a computer or a smartphone, which in turn may be connected to the Tolerance Scoring Unit 1810. Ideally, the I/O device has a display, wherein the output, i.e., whether the protein of interest or the binding pocket therein is intolerant to variation, is displayed.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

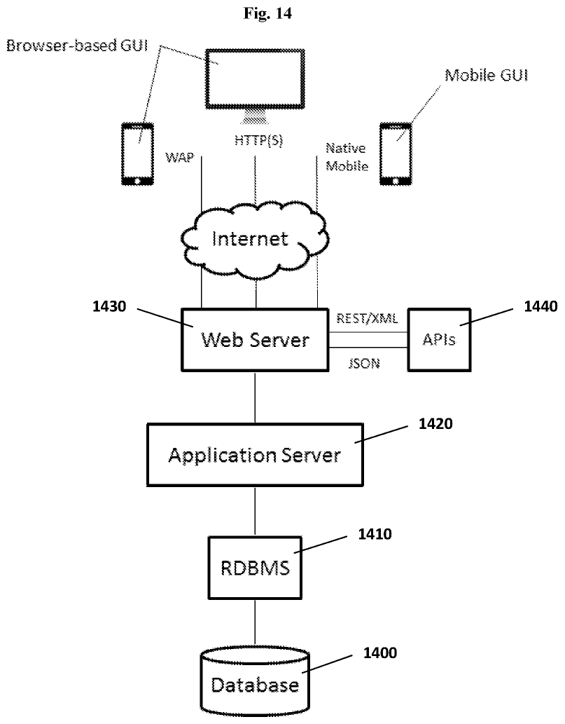

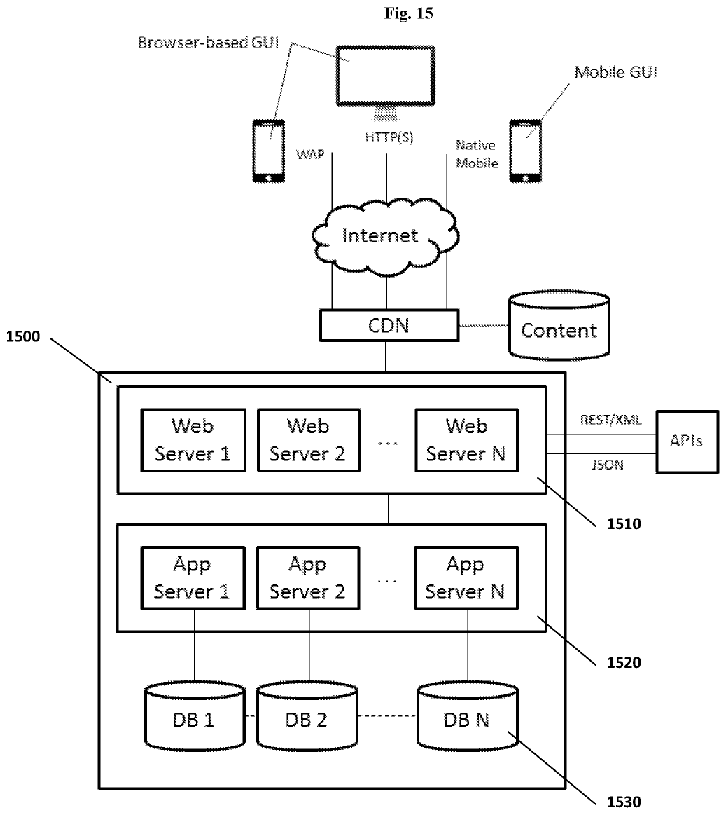

Referring to FIG. 14, in a particular embodiment, an application provision system comprises one or more databases 1400 accessed by a relational database management system (RDBMS) 1410. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1420 (such as Java servers, .NET servers, PHP servers, and the like) and one or more web servers 1430 (such as Apache, IIS, GWS and the like). The web server(s) optionally expose one or more web services via app application programming interfaces (APIs) 1440. Via a network, such as the Internet, the system provides browser-based and/or mobile native user interfaces.

Referring to FIG. 15, in a particular embodiment, an application provision system alternatively has a distributed, cloud-based architecture 1500 and comprises elastically load balanced, auto-scaling web server resources 1510 and application server resources 520 as well synchronously replicated databases 1530.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. In addition, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of protein structure, nucleic acid sequence data, and 3DTS scores, either by amino acid, protein feature or entire protein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the software applications, systems, and methods described herein and are not meant to be limiting in any way.

### Example 1—Determination of a Three-Dimensional Tolerance Score (3DTS)

### Sequence and Protein Structure Data

A set of 7,794 deep-sequenced unrelated whole human genomes (an extension from previous work), and 123,136 exomes and 15,496 whole human genomes from gnomAD (gnomad(dot)broadinstitute(dot)org/) was used in development. All data was aligned or lifted to human reference hg38. Variants from our set were included if they fell within the extended confidence region (as previously described) while gnomAD variant calls were included if they were annotated as “PASS”, and could be lifted over to hg38. Total call counts were derived from the sequence coverage files of gnomAD or from internal datasets. Structural and functional feature annotations were taken from the respective Uniprot text files (uniprot(dot)org; downloaded: April 2018) that were cross-referenced to Genecode. Features include secondary structure elements (helix (HELIX), beta strand (STRAND), turn (TURN)) and others: binding site (BINDING), modified residue (MOD_RES), mutagenesis (MUTAGEN), region (REGION), motif (MOTIF), nucleotide binding (NP_BIND), natural variant (VAR_SEQ), active site (ACT_SITE), metal binding (METAL), disulfide bond (DISULFID), glycosylation (CARBOHYD), site (SITE), peptide (PEPTIDE), domain (DOMAIN), DNA binding (DNA_BIND), repeat (REPEAT), signal (SIGNAL), cross-link (CROSSLNK), lipidation (LIPID), propeptide (PROPEP), calcium binding (CA_BIND), topological domain (TOPO_DOM), zinc finger (ZN_FING), coiled-coil (COILED), compositional bias (COMPBIAS), transmembrane (TRANSMEM), intramembrane (INTRAMEM), transit peptide (TRANSIT), and non-standard residue (NON_STD). More specific definitions of these features are provided at uniprot(dot)org/help/sequence_annotation. Pathogenic variation data was sourced from Clinvar and HGMD. Selected Clinvar variants were tagged as (likely-)pathogenic and have 1 or more stars. Selected HGMD variants were tagged as DM and High. Any pathogenic variants overlapping a variant annotated as benign with 1 or more stars in Clinvar were filtered out. We used the transcripts and the gene model of Gencode version 26. We used pairwise global sequence alignment to align the Uniprot amino acid sequence to the Gencode transcript sequence (after translating them to amino acids). The pairwise alignment algorithm was parameterized with the Blosum62 matrix, with a gap open penalty of 5 and a gap extension penalty of 1. Very large features, which mapped to more than 300 nucleotides, were excluded, as these would not provide information about local structures.

X-ray structure data from the Protein Data Bank (PDB; www(doOrcsb(dot)org) were used if they were linked within the Uniprot text files. We used a pairwise global sequence alignment approach to align the Uniprot amino acid sequence to the amino sequence retrieved from the macromolecular Crystallographic Information File (mmCIF). Alignment parameters were set as above. The first author-defined biological assembly in the mmCIF file was used, when defined. In the cases in which it was not defined, the first biological assembly listed was used. In the case of the RING-domain of BRCA1, we used the NMR structure closest to the average as defined by the mmCIF file. The Pymol molecular visualization system (The PyMOL Molecular Graphics System, Version 1.8 Schrodinger, LLC.) was used to identify any residue within 5 Å of a defined Uniprot feature (also referred to as a “3D-site”). In the case of SWISSMODEL 48, human proteome metadata and coordinates data were downloaded from the SWISSMODEL Repository (UniprotKB release 2018_05) and were included if the QMEAN Z-score >−4. Secondary structures in the models were defined with DSSP-2.0.4 (Touw et al., Nucleic Acids Res 43, D364-8, 2015); available via the FTP server cmbi(dot)ru(dot)nl/pub/software/dssp/) and 3D sites were defined using the secondary structure elements.

### Quantification of Depletion of Variation and 3D-Tolerance Score

Variation at genomic loci is modeled with independent Bernoulli trials. At loci i in individual j a variation happened with probability p. We assume that certain variants are incompatible with life in which case the variant is missing from the sample. Thus, the compound probability of observing a variant at a locus in an individual is p*s, where p is not specific to the variant, but is a genome wide mutation rate and s is specific to a variant with the interpretation of the probability that the variant is lethal. If s=0 then the genomic locus is completely depleted of variants, while if s=1 then all variants are present as expected by the generic mutation rate of p. This model is not valid for common variants, but describes the process of a rare de novo mutations. In particular this model ignores: inheritance and relationship of individuals, linkage of variants by sharing a haplotype, allele frequency and zygosity. We estimate the value of s because s is a proxy of the strength of purifying selection on the genomic locus.

A nucleotide on the ancestral chromosome can change into three other nucleotides, not all of them causing a non-synonymous mutation. We incorporate this into our model by extending it with the probability (b) that either of the three non-ancestral alleles lead to amino acid change. The value of b is derived from the genetic code and from the amino acid sequence of the transcript. With this extension, the probability of a mutation is p*s*b.

To maximize power, we aggregate variants both by samples and by sets of loci induced by protein structure. Thus we write the probability of observing at least one variation at a given locus in R individuals: 1−exp(−p*s*b*R). The latter follows from the Poisson approximation of the binomial distribution: the sum of the number of successes in R Bernoulli trials with the same parameter is a binomial distribution, which can be well approximated with the Poisson distribution if R is large. The expression follows if we express ‘at least one’ as ‘not zero’.

To aggregate by different loci we treat each loci as a Bernoulli trial with parameter 1−exp(−p*s*b*R). These parameters are however different for each loci; thus, the sum of the number of successful trials is described with the Poisson binomial distribution. The expected value of the Poisson binomial distribution is the sum of its parameters (in our case sum(1−exp(−p*s*b*R)), its density and distribution function may be approximated with the Poisson distribution (Le Cam's theorem) or computed using Fourier transformations. For efficiency, we use the Poisson approximation.

For instance, in a given locus, the number of called positions across a population can vary therefore necessitating the index l on the parameter R. Each locus has different bₗ and Rₗ parameters thus aggregating K>1 loci into a single unit is a sum of Bernoulli trials of heterogeneous parameters. Thus, we approximate again with a Poisson distribution following Le Cam's theorem. The final likelihood function of our model is P (observed k variants in K loci among R samples|pₗ, s, bₗ)=Poi(k, Σₗ^{K} 1−exp(−pₗ s bₗ Rₗ)), wherein pₗ is the expected number of mutations between the reference genome and the sample assuming that mutations at the locus are neutral; bₗ is how likely a nucleotide change leads to a missense variation and sₗ is an adjustment factor; sₗ is the parameter of interest, which in its two extremes is either 0 if all variation happening at the locus is deleterious or 1 if none is.

The above model has two nuisance parameters bₗ and pₗ. We know bₗ from the genetic code and reading frame. We learn pₗ by two approaches—(1) from the chromosome-specific non-coding variation data (“constant mutation rate”); and (2) from the nucleotide context dependent chromosome-specific non-coding variation data (“heptamer rate”). To these data, we apply the previously described model and find the value or values of pₗ which maximizes the likelihood. To do so we set s to 1 assuming that mutations in these regions do not confer deleteriousness, and encode our constraint that we would like s to quantify the difference between the coding region of interest and the neutral territories. We calculate nucleotide context dependent estimates by partitioning all intergenic loci by the 7-mer which symmetrically spans the locus. We then find a maximum likelihood estimate specific for each heptamer. It should be noted that 98.5% of 3D sites do not contain a common missense variant (AF>0.05) and would not be affected by the incorporation of an allele frequency term—this is the rationale to favoring a model that uses context (k-mer) expectation of variation.

Recognizing that the problem is one dimensional, numerical integration (Gauss-Legendre quadrature and importance sampling) is used to calculate the posterior mean on s with a uniform U(0,1) prior. Thus, downstream analysis of the posterior mean of s as the 3D tolerance score (3DTS) is provided.

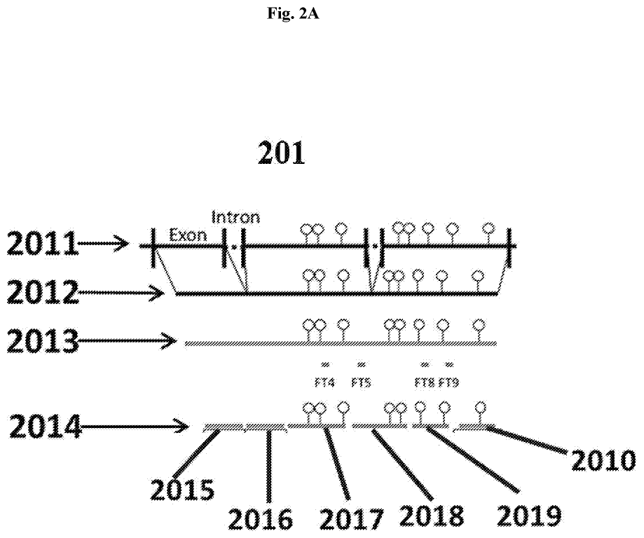

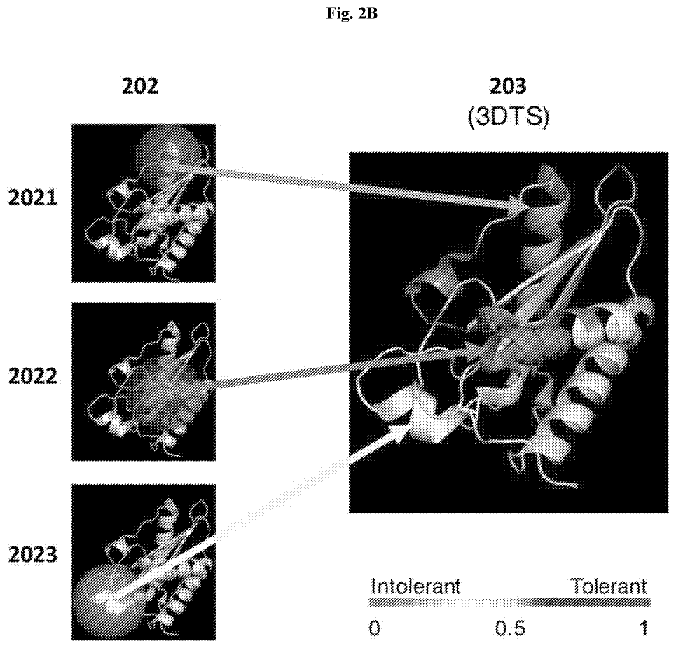

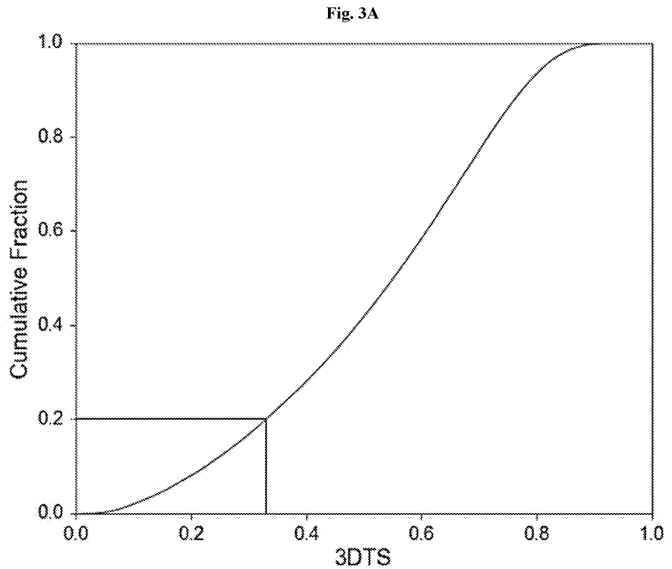

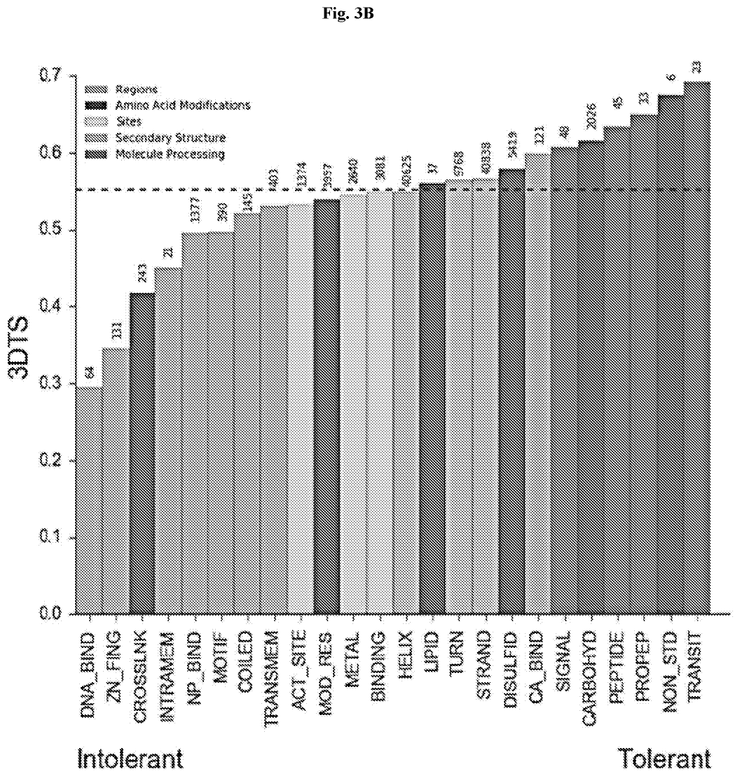

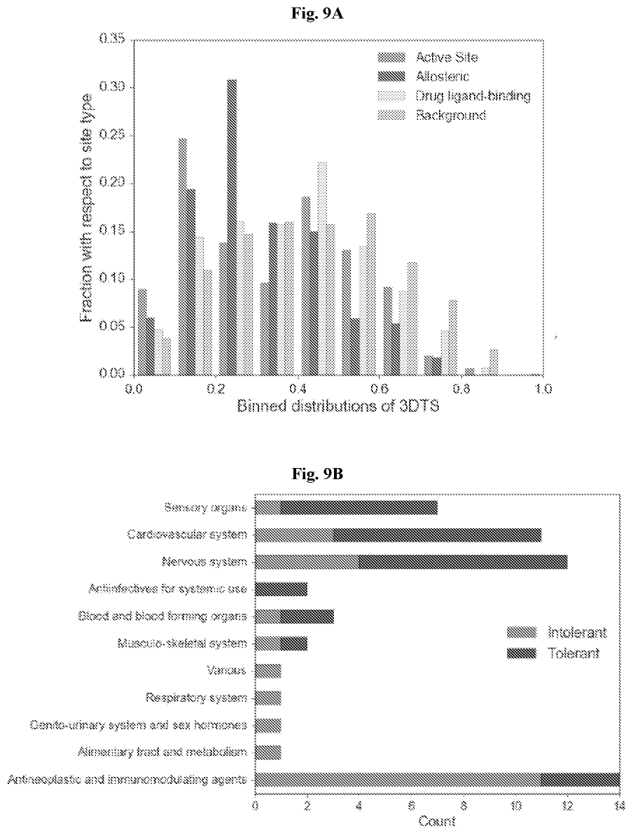

In calculation of DTS scores, constant mutation rate is primarily used, except when comparing the effects of varying these parameters, as in FIG. 4D, and when describing the optimal 3DTS model in FIG. 4E. The “Structure” feature set, which uses secondary structure elements only, was used in the cases of FIG. 4A, FIG. 4B and FIG. 4F. In the case of FIG. 2A, FIG. 2B, FIG. 3A, FIG. 3B, FIG. 7, FIG. 9A and FIG. 9B, all features were used. The optimal model was defined by the highest Pearson r² value that showed correct directionality in correlation and that had both significant Pearson and Spearman p-values (p<0.05) (Table 1). In the case of TPMT, which did not meet the Pearson p-value threshold of significance, the model showing a significant Spearman p-value was considered optimal.

Above we described the probability of observing at least one non-synonymous variant across a set of loci and a set of R samples assuming parameters p and s. We continue by estimating s from the available data. We employ two approaches: first, we assume a global, genome wide constant value for the mutation rate parameter p, while in the second approach we estimate p locally for each protein.

### Assuming a Genome Wide Mutation Rate

One approach to estimate a global mutation rate (or constant mutation rate) is by numerically fitting the observed number of synonymous variants across all mapped proteins to the expected value of the number of synonymous variants fixing s=1. The estimated value is 2.5×10⁻⁶ and is treated as a constant. We numerically calculate the posterior distribution of the remaining single s parameter by assuming a uniform prior between 0 and 1. In equations:

P(observed k variants in K loci among R samples|s)=Poisson distribution (k, sum over K(1−exp(−p*s*b*R))), the likelihood function using Le Cam's approximation P(s)=1, uniform prior over 0-1

P(s|observed k variants in K locus among R samples)=Likelihood*prior/integrate over 0-1 (likelihood*prior), the posterior

We summarize the posterior distribution of s by its expected value (mean), which we assign to each protein feature and refer to it as 3DTS score.

### Locally Estimating the Mutation Rate for Each Protein

The first approach does not accurately reflect a local mutation rate because the biological mutation rate varies over genomic regions (e.g., different localities), and because the rate of variant discovery varies as well, with larger sets defining higher mutation rates especially for rare variants. Here we estimate a local mutation rate parameter from data across the whole protein chain, then proceed as described above.

### Other Approaches to Summarize the Posterior Distribution of s:

The mean of the posterior distribution of s may be interpreted as the estimate of the probability that a non-synonymous variant is lethal. However, in case of small protein features and low data availability, it tends towards 0.5 due to the choice of the uniform prior on 0-1.

Taking advantage of the low dimensionality, we used numerical quadratures to evaluate the integrals. Statistical distributions were evaluated using the jdistlib library. (jdistlib(dot)sourceforge(dot)net/).

### Functional Data and Pathogenicity Scores

Functional in vitro data for PPARG was sourced from Majithia et al. (Majithia (supra). The integrated functional scores available through miter(dot)broadinstitute.org/ (Data version 1.0) were used. Only those scores linked to amino acid changes resulting from a single nucleotide variation were included in this analysis.

Functional in vitro data for the RING domain of BRCA1 were sourced from Starita et al. (supra). Known homology directed repair (HDR) rescue scores from the HDR rescue assay were used when available, otherwise predicted values were used. Only those scores linked to amino acid changes resulting from a single nucleotide variation were used in the comparison with 3DTS.

Deep mutational scanning data are available for the following additional proteins MAPK1/ERK2 (Brenan et al., Cell Rep 17, 1171-1183, 2016), p53 (Kato et al., PNAS USA 100, 8424-9, 2003), PTEN and TPMT (Matreyek et al., Nat Genet 50, 874-882, 2018), UBE2I , SUMO1, TPK1, CALM1, CALM2 and CALM3 (Weile et al., Mol Syst Biol 13, 957, 2017) and two single protein domains of BRCA1 (the RING domain) and YAP65 (the WW domain)(Fowler et al., Nat Methods 7, 741-6, 2010; Starita et al., Genetics 200, 413-22, 2015).

For datasource, MAPK1/ERK2 data was sourced from Supplementary Table S1 in Brenan et al. (supra). The log-fold2 change of ERK2 mutant abundance following DOX induction relative to the mutant abundance in the early time point for missense variants caused by SNVs were averaged for an amino acid site and then averaged across a 3D site for the comparison with 3DTS. PTEN and TPMT data were sourced from Supplementary Datasets 3 and 4 in Matreyek et al. (supra). The “score” columns were averaged across each 3D site and compared to 3DTS. UBE2I, SUMO1, TPK1, CALM1, and CALM2 data were sourced from Dataset EV1 in reference Weile et al. (supra) The “joint.score” column was averaged across an amino acid position for missense variants and then averaged across a 3D site and compared to 3DTS. Since quantitative information on p53 could not be retrieved at the residue/feature level from the original publication, p53 was not scored. Similarly, CALM3 was not scored because no structure was available for the protein; and RING domain of BRCA1 and the WW domain in YAP65 were not scored since only limited data is available for these domains.

For comparative analysis (e.g., to compare the systems and methods of the disclosure to art-existing methods), method data were sourced from dbNSFPv3.5a (Dong et al. (supra); Liu et al., Hum Mutat 37, 235-41, 2016) except for EVmutation data (see Hopf et al., supra), which were sourced from (marks(dot)hms(dot)harvard(dot)edu/evmutation/humanproteins.html). The data fields used were: “CADD_phred” (CADD), “MutationAssessor_score” (MUTATIONACCESSOR), “fathmm-MKL_coding_score” (FATHMM-MKL), “integrated_fitCons_score” (FITCONS), “DANN_score” (DANN), “MetaSVM_score” (METASVM), “MetaLR_score” (METALR), “GenoCanyon_score” (GENOCANYON), “Eigen-PC-phred” (EIGEN), “M-CAP_score” (M-CAP), “REVEL_score” (REVEL), “phyloP100way_vertebrate” (PHYLOP_vertebrate), “phyloP20way_mammalian” (PHYLOP_mammalian), “phastCons100way_vertebrate” (PHASTCONS_vertebrate), “phastCons20way_mammalian” (PHASTCONS_mammalian), “GERP++_RS” (GERP), “SiPhy_29way_logOdds” (SIPHY) and “prediction_epistatic” (EVMUTATION). Scores resulting in missense variants were averaged across a nucleotide (where applicable), then an amino acid position and lastly across a 3D site. 3D sites were defined by the features showing the lowest 3DTS value for an amino acid position and correlations were made over available data.

### Variant Distance Data and Analyses

Distance-based quantification was performed using Pymol. Pathogenic variation data was sourced from Clinvar (July 2016) and HGMD (first quarter 2016, R1). Selected Clinvar variants had to be tagged as (likely-)pathogenic and have 1 or more stars. Selected HGMD variants had to be tagged as DM and High. Any pathogenic variants overlapping a variant annotated as benign with 1 or more stars in Clinvar were filtered out. Structures were included in this analysis if at least 70% of the total canonical protein length was covered and at least one pathogenic missense variant was present.

### Drug Ligand Data Set and Analyses

A set of structures defined as therapeutic targets of FDA-approved drugs was used. Therapeutic targets were taken from the supplementary information of Santos et al. (Nat Rev Drug Discov 16, 19-34, 2017). Of 667 non-redundant Uniprot entries, 361 contained some structural information and 100 contained proteins where the sequence length of the structure defined by Uniprot covered at least 80% of the canonical Uniprot sequence. Ninety-four of these 100 proteins were mapped to the genome using Gencode version 26. These 94 proteins were examined for the presence of the corresponding bound therapeutic molecule or analog in a structure; when not found, homologous structures containing these molecules were superimposed, resulting in 48 structures with their corresponding “bound” therapeutic molecule (for a list of these structures and their “bound” ligands, see Table 2). Ligand binding sites were defined as those residues within 5 Å of any of the bound therapeutic molecule residues. The lowest 3DTS value was assigned to each of these residues in cases of overlapping 3D-sites.

### Anatomical Therapeutic Chemical (ATC) Classification System Data and Analyses

Drug-liganded molecules (as identified in the above Drug Ligand Data Analyses section) were assigned to their ATC codes using the supplementary information of Santos et al. (supra). For each structure, a non-redundant list of top-level ATC code was included for all bound drugs. In cases in which no ATC code was found, the code was inferred either based on indication (when available) or based on indirect effect. In cases where the structure had multiple chains contributing to the ligand-binding site, the median score was used in defining tolerance.

### Allosteric Data Set and Analyses

The XML, data of the Allosteric Database (Release 3.06) was downloaded and parsed with custom Python scripts. Data was used if the field “Organism Latin” was equal to “Homo sapiens”, any of the allosteric counts (“Allosteric_Activator_Count”, “Allosteric_Inhibitor_Count”, or “Allosteric_Regulator_Count”) had a value of at least one, and “Site_Detail” contained at least one defined amino acid. Of the resultant fifty-four entries, fifty structures were mapped where every allosteric residue had a 3DTS value. The lowest 3DTS value was assigned in cases of overlapping 3D-sites. These structures were used in the downstream analysis (for a list of these structures and molecules binding thereto, see Table 2).

### Active Site Data Set and Analyses

A non-redundant list of protein active sites was included for those structures found in the Drug Ligand Data Set and the Allosteric Data Set. Active sites were defined based on the 5 Å context of the “ACT_SITE” feature(s) defined in Uniprot (i.e., “ACT_SITE” 3D-sites).

### Unique, Non-Overlapping 3D-Intolerant Site Analyses

Structures from the Drug Ligand Data Set and Allosteric Data Set were used. A 3D-site was defined as intolerant if the 3DTS value was in the 20th percentile proteome-wide (3DTS value<0.33). 3D-intolerant sites were joined if at least one residue overlapped within a chain. For homomeric chains, two intolerant sites were considered unique if no residue in the primary structure was shared. In cases where chains representing the same protein differed in the number of unique, non-overlapping 3D-intolerant sites, the maximum number of 3D-intolerant sites was chosen. Statistics

Plots were produced using the Seaborn (seaborn(dot)pydata(dot)org) and Matplotlib (matplotlib(dot)org) libraries in Python. Statistics were calculated using the NUMPY (www(dot)numpy(dot)org) and SCIPY (www(dot)scipy(dot)org) libraries in Python and in house statistical software in Scala.

To understand variation in the structural proteome, we first identified 26,593 structures associated with 4,390 Uniprot entries that fulfilled our inclusion criteria: x-ray crystal structures with a defined resolution, a minimum chain length greater than 10 amino acids and at least 80% identity between the aligned matches of the Uniprot canonical sequence and the PDB structure. Given the multiplicity of possible structures for the 4,390 proteins, we chose as representative, the structure with the most scored Uniprot features. In total, we mapped 139,535 Uniprot features to the structures, and extracted a 3-dimensional context by defining a 5-Angstrom radius space for each feature; hereafter referred to as a “3D-site”. We identified 481,708 missense variants for these proteins from the analysis of 146,426 individuals' exomes. From these contextualized data, we constructed a model that describes functional constraints in three-dimensional protein structures (FIG. 2). As shown in step 201 of FIG. 2A, missense variation data from genome and exome sequencing projects 2011 (missense mutations shown by circles) are mapped to their corresponding canonical amino acid sequences 2012. A mapping between protein crystal structure 2013 and the corresponding amino acid sequence is created 2014 (including protein features 2015-2010). In step 202 of FIG. 2B, features extracted from Uniprot are then mapped to the 3D structure. Using these features 2021, 2022, and 2023 as reference points, a 3D context is constructed and the corresponding genetic data is extracted. The 3DTS for each feature region is generated from this information. The 3DTS scores can be ranked and the corresponding tolerance ranks (or scores) can be projected back onto the 3D structure 203. The strength of constraint (intolerance) was reflected in a 3-dimensional tolerance score (3DTS) that summarized the differences between observation and expectation in genetic variation at the level of 3D-sites.

For the representative set of structures for the 4,390 proteins, we describe the distribution of 3DTS values in FIG. 3A. In total, 2,642 proteins had at least one intolerant 3D-site defined at the 20th percentile (3DTS=0.33, approximately 70% depletion of observed over expected missense variation). The most intolerant 3D-sites corresponded to DNA binding sites, zinc fingers, and cross-linkages, while the most tolerant 3D-sites included transit peptides, non-standard residues (i.e., selenocysteines), and propeptides. Structural features (helix, turn, strand) showed median 3DTS values close to the median proteome-wide. As shown in FIG. 3B. The precise interpretation of 3DTS values required the assessment of functional consequences of amino acid changes in intolerant versus tolerant 3D-sites. However, a challenge of functional testing proteome-wide is the requirement of cellular assays that are disease and gene relevant, robust, and scalable—a serious limitation that explains that to this date, the experimental characterization of all possible missense variants in a mammalian gene has been limited to one full protein, PPARG, and two single protein domains of BRCA1 (the RING domain) and YAP65 (the WW domain). We therefore sought to validate 3DTS against the available functional data for these proteins and domains. In the case of the WW domain in YAP65, positional functional data were not easily accessible and the domain represented a set of only 25 amino acid positions; therefore it was not assessed.

### Example 2—Determination of a Three-Dimensional Tolerance Score (3DTS) for Human PPARG and Comparison with in Vitro Results

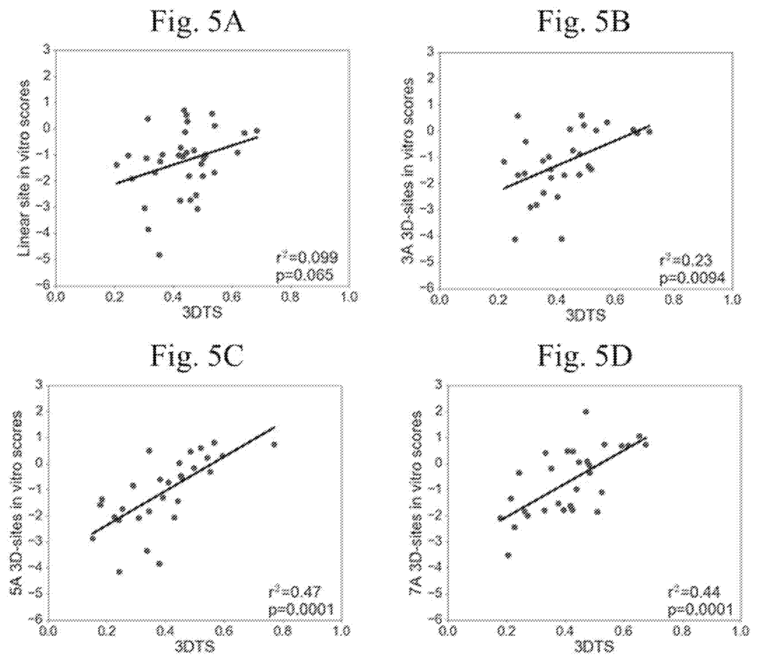

PPARG is a drug target for thiazolidinediones and newer partial PPARG modulators used in the treatment of diabetes. PPARG exemplifies the challenge of classifying newly identified variants even in a well-studied protein implicated in disease. In the original work, functional interpretation of PPARG variants required the construction of a cDNA library consisting of all possible amino acid substitutions in the protein. The library was introduced into human macrophages edited to lack the endogenous PPARG, and stimulated with PPARG agonists to trigger the expression of CD36, a canonical target of PPARG. Sorted CD36+ and CD36− cell populations were sequenced to determine the distribution of each PPARG variant in relation to CD36 activity. We showed a strong correlation (r2=0.47, p=0.0001) between the 3D-sites defined by 3DTS and the functional scores described in Majithia et al. Specifically, both the in vitro score shown in FIG. 4A and the in silico score shown in FIG. 4B identified the DNA-binding and ligand binding sites as intolerant to missense variation, while the hinge domain reflected increased tolerance to missense variation as shown in FIG. 4A and B. The 5 Å FIG. 5C context also showed stronger correlations than the linear features (a 0 Å context) FIG. 5A, a 3 Å context FIG. 5B, or a 7 Å context FIG. 5D. Additionally, Majithia et al. indicated that their transgene library may not have detected all possible functional effects of coding variation, suggesting that the concordance of r²=0.47 as shown in FIG. 4C between in vitro and in silico readouts should be interpreted as conservative.

### Example 3—Analysis of Other Proteins with Existing Deep Mutational Scanning Data

The methodology implemented in Example 2 (above) was applied to other proteins of interest for which existing mutational scanning data is available. These include, calmodulin 1 (CALM1), calmodulin 2 (CALM2), mitogen-activated protein kinase 1 (MAPK1 or ERK2), peroxisome proliferator activated receptor gamma (PPARG), phosphatase and tensin homolog (PTEN), small ubiquitin-like modifier 1 (SUMO1), thiamin pyrophosphokinase 1 (TPK1), thiopurine s-methyltransferase (TPMT), and ubiquitin conjugating enzyme E2 I (UBE2I). Results are shown in FIG. 4D, showing distributions of Pearson r² values for all structures (ranging from 0 to 0.72 for CALM1, 0 to 0.54 for CALM2, 0.02 to 0.33 for ERK2, 0.17 to 0.41 for PPARG, 0.21 to 0.39 for PTEN, 0 to 0.83 for SUMO1, 0.13 to 0.22 for TPK1, 0.09 to 0.17 for TPMT, and 0 to 0.62 for UBE2I) that cover at least 70% of the canonical isoform under four different 3DTS conditions: two different sets of 3D features and two different models of rate variation. Importantly, different structures for the same protein differ in the correlation value—the median r² and the distributions tend to be large both within and between conditions and genes. These variations could occur for a variety of reasons such as alternative protein interaction partners, different structural coverages of the protein, varied crystallization conditions, etc. We speculate that 3DTS might serve to identify functionally relevant conformations for a given protein; i.e., for a protein with multiple available structures, the best correlations may represent the most parsimonious and functionally plausible structures. Data regarding the optimal structures are available in Table 1.

TABLE 1
Data for optimal structures of the proteins

| | Gene Name | | | | | | | | |
|---|---|---|---|---|---|---|---|---|---|
| | CALM1 | SUMO1 | UBE21 | TPK1 | CALM2 | PTEN | TPMT | PPARG | ERK2 |
| Uniprot | P0DP23 | P63165 | P63279 | Q9H3S4 | P0DP24 | P60484 | P51580 | P37231 | P28482 |
| Entry | | | | | | | | | |
| PDB, | 3G43, B | 3KYD, D | 3A4S, A | 3S4Y, A | 5WSV, A | 5BZZ, A | 2H11, A | 3DZY, D | 5V62, A |
| Chain | | | | | | | | | |
| Mutation | Heptamer | Heptamer | Heptamer | Constant | Constant | Constant | Heptamer | Constant | Constant |
| Rate | | | | | | | | | |
| Estimate | | | | | | | | | |
| Feature | All | Structure | Structure | All | All | Structure | Structure | Structure | All |
| Set | | only | only | | | only | only | only | |
| In vitro | (i) | As | As | As | As | (i) site- | As | (i) cDNA | (i) pooled, |
| analysis | mutagenesis; | previous | previous | previous | previous | saturation | previous | library | virally- |
| | (ii) | | | | | mutagenesis | | consisting | delivered |
| | generation | | | | | libraries | | of all | cDNA |
| | of a | | | | | using | | possible | expression |
| | variant | | | | | inverse | | amino acid | library, (ii) |
| | library; | | | | | PCR, (ii) | | substitutions, | transduction |
| | (iii) | | | | | libraries | | (ii) into | in cells |
| | selection | | | | | recombined | | human | with |
| | of | | | | | in | | macrophages | constitutive |
| | functional | | | | | engineered | | edited to | activation |
| | variants in | | | | | HEK 293T | | lack the | of ERK1/2, |
| | S. | | | | | cells, (iii) | | endogenous | (iii) |
| | cerevisiae | | | | | FACS by | | PPARG, | induced |
| | functional | | | | | EGFP: | | (iii) | mutant |
| | complemen- | | | | | mCherry, | | stimulated | library |
| | tation | | | | | (iv) | | to trigger | expression |
| | assay (iv) | | | | | Sorted | | the | with |
| | scoring of | | | | | library | | expression | doxycycline, |
| | the | | | | | genomic | | of CD36, | (iv) |
| | selection | | | | | DNA | | (iv) sorted | quantified |
| | results to | | | | | preparation, | | cell | the relative |
| | produce a | | | | | barcode | | populations | abundance |
| | sequence- | | | | | amplification | | sequenced | of each |
| | function | | | | | and | | to | mutant via |
| | map | | | | | sequencing. | | determine | massively- |
| | | | | | | | | the | parallel |
| | | | | | | | | distribution | sequencing |
| | | | | | | | | of each | |
| | | | | | | | | PPARG | |
| | | | | | | | | variant in | |
| | | | | | | | | relation to | |
| | | | | | | | | CD36 | |
| | | | | | | | | activity. | |
| Pearson | 0.72268 | 0.62416 | 0.62219 | 0.22126 | 0.4125 | 0.36721 | 0.14743 | 0.41029 | 0.31101 |
| r**2 | | | | | | | | | |
| Pearson | 0.03202 | 0.00654 | 0.00135 | 0.01531 | 0.02432 | 0.00063 | 0.10459 | 0.00003 | 0.00023 |
| p-value | | | | | | | | | |
| Spearman | 0.88898 | 0.59243 | 0.39234 | 0.33898 | 0.40496 | 0.46288 | 0.37704 | 0.50032 | 0.4835 |
| r**2 | | | | | | | | | |
| Spearman | 0.0048 | 0.00922 | 0.02199 | 0.00181 | 0.0261 | 0.00007 | 0.00516 | 0.000001 | 0.000001 |
| p-value | | | | | | | | | |
| PUBMED | 29269382 | 29269382 | 29269382 | 29269382 | 29269382 | 29785012 | 29785012 | 29455857 | 27760319 |
| Ref | | | | | | | | | |

Next, the functional predictive capability of 3DTS was compared with 21 published scores: CADD (Kircher et al., Nat Genet 46, 310-5, 2014), SIFT (Kumar et al., Nat Protoc 4, 1073-81, 2009), PROVEAN (Choi et al., PLoS One 7, e46688, 2012), FATHMM (Shihab et al., Hum Mutat 34, 57-65, 2013), MUTATIONASSESSOR (Reva et al., Genome Biol 8, R232, 2007), FATHMM-MKL (Shihab et al., Bioinformatics 31, 1536-43, 2015), FITCONS (Gulko et al., Nat Genet 47, 276-83, 2015), DANN (Quang et al., Bioinformatics 31, 761-3, 2015), METASVM/METALR (Dong et al., Hum Mol Genet 24, 2125-37, 2015), GENOCANYON (Lu et al., Sci Rep 5, 10576, 2015), Eigen-PC (Ionita-Laza et al., Nat Genet 48, 214-20, 2016), M-CAP (Jagadeesh et al., Nat Genet 48, 1581-1586, 2016), REVEL (Ionnidis et al., Am J Hum Genet 99, 877-885, 2016), PHYLOP (Pollard et al., Genome Res 20, 110-21, 2010), PHASTCONS (Siepel et al. Genome Res 15, 1034-50, 2005), GERP++7, SIPHY (Garber et al., Bioinformatics 25, i54-62, 2009), EVMUTATION (Hopf et al., Nat Biotechnol 35, 128-135, 2017). These various scores trained under a range of assumptions, most commonly interspecies conservation, co-evolution, and pathogenicity. Overall, 3DTS performs comparably or better than these other methods in the 3D space (FIG. 4E). The availability of multiple proteins with deep mutational screening data also supported a more formal assessment of the effect of varying the size of the 3D sites and confirming the general validity of the use of the 5 A radius.

Next, the aforementioned evaluation was extended to a large corpus of functional readouts for 1,026 proteins for which shallow mutational information was available. The median 3DTS score for 4,428 3D functional sites (those that carry an experimentally tested “loss of function” variant) is lower than the proteome background (Kolmogorov-Smirnov two-sided test pvalue=3.7E-42), which may yet include undescribed functional sites. Importantly, at any level of global gene essentiality, functional sites are systematically more constrained than the rest of the protein (FIG. 4F). In summary, the results show that in silico 3DTS values provide functional prediction without engaging in extensive and time-consuming in vitro assays and dedicated functional readouts. This is critical, given the paucity of human proteins that have been subjected to deep mutational scanning and functional testing.

### Example 4—Determination of a Three-Dimensional Tolerance Score (3DTS) for Human BRCA1 and Comparison with in Vitro Results

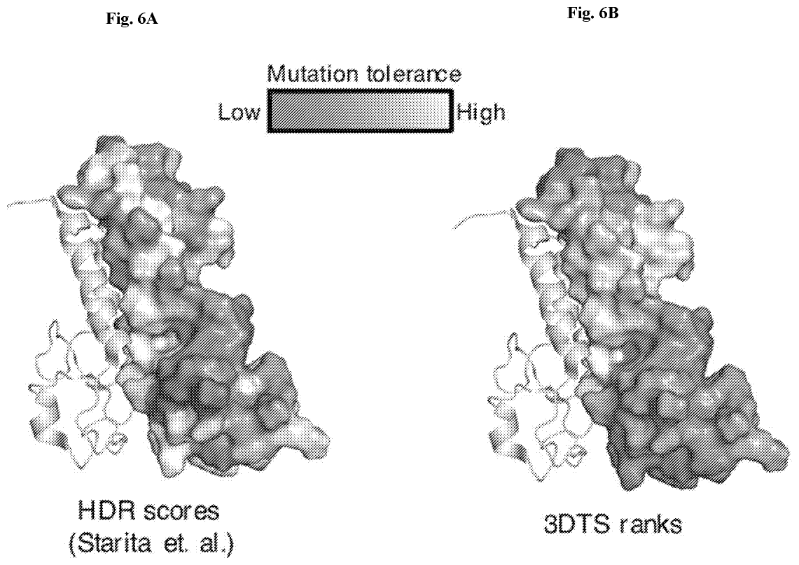

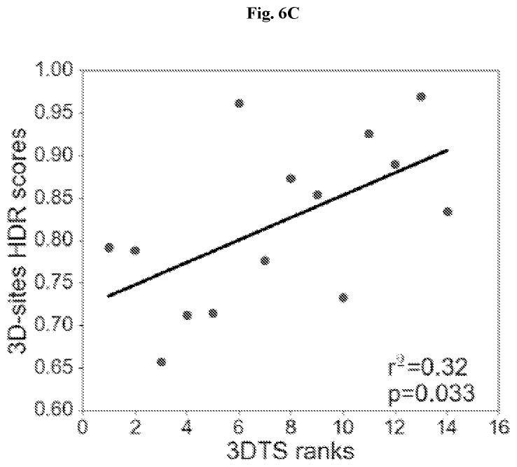

Another example uses BRCA1; an informative exercise because the approach is validated for only one of the structural domains (RING). The RING domain represents only 5% of the canonical BRCA1 protein; however, 58% of the pathogenic missense substitutions occur within this domain. See Starita et al. (Genetics 200, 413-22, 2015). In the original work, functional analysis of the RING domain required testing for two functions: BRCA1 E3 ligase activity in phage display assays, and interaction with BARD1 in yeast two-hybrid assays. The combination of these two molecular functions into a larger biological function (Graphically represented in FIG. 6A), homology directed repair, resulted in a concordance of r²=0.32, p=0.033 as shown in FIG. 6C, with the rank values of 3DTS (graphically represented in FIG. 6B). The zinc-binding sites showed the greatest intolerance in this structure. In summary, the in silico 3DTS values recapitulate in vitro functional data without engaging in complex assays that require extensive and time-consuming in vitro assays and dedicated functional readouts.

### Example 5—Determination of Pathogenic Variants using a Three-Dimensional Tolerance Score (3DTS)

Predicting functionally intolerant 3D sites, and the distribution of variants with respect to these sites, may have several practical applications. For example, variants within intolerant sites may carry phenotypic consequences (i.e., pathogenicity). We thus aimed at establishing the association between 3D intolerance to variation and pathogenicity of variants. We identified 192 structures with at least one pathogenic missense variant (3081 total variants) and at least one common (allele frequency>1%) missense variant (373 total variants). Shown in FIG. 7 are enrichment lines on a per Angstrom basis outside of the most intolerant site. The line 701 represents the enrichment of pathogenic missense variants over common (allele frequency>1%) missense variants. The distance between the closest atoms of the most intolerant feature and each variant were measured. In this set, the greatest enrichment of pathogenic relative to common variants appeared within the most intolerant site (2.3-fold enrichment) and another peak in enrichment was seen within ˜6-14 Å of the most intolerant site 703.

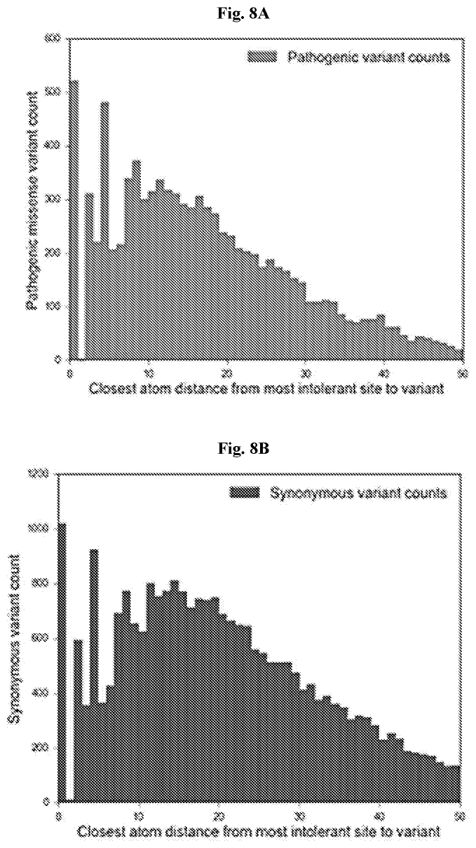

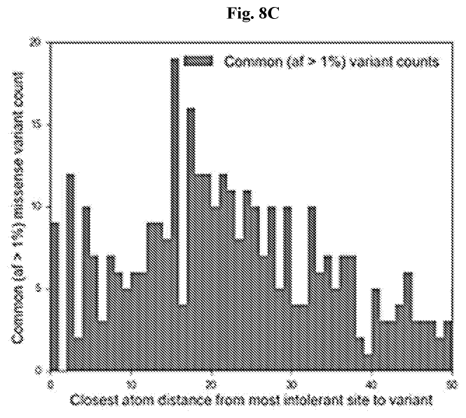

Due to the scarcity of common missense variants, we also used synonymous variants as a proxy for neutral variation, which increased the number of available structures to 438 and the number of pathogenic missense variants to 9,531, leveraging a total of 26,229 synonymous variants. The line 702 indicates the enrichment of pathogenic missense variants over synonymous variants. In this set, the greatest enrichment of pathogenic variants was observed ˜4-9 Å away from the most intolerant site Raw counts for each variant type with respect to distances are presented in FIG. 8A-8C. The reduction of number of counts at very close distances may be related to the spatial restrictions on distances smaller than a van der Waals contact.

The enrichment of pathogenic variation diminishes with distance. Distance mapping of pathogenic variants shows the highest enrichment of pathogenic to benign variants to be near and within the most intolerant features defined by 3DTS.

### Example 6—Determination of Druggable Targets using a Three-Dimensional Tolerance Score (3DTS)

Another application of the present work could involve prioritization of drug target sites. Protein structure-based methods are now routinely used at all stages of drug development, from target identification to lead optimization. Central to all structure-based discovery approaches is the knowledge of the 3D structure of the target protein or complex because the structure and dynamics of the target determine which ligands it binds. The characterization of human-specific intolerant sites and tolerance to genetic variation can be used to parse structural information to define active sites, but also to define functionally important topographically distinct sites that can support allosteric interactions.

We analyzed the 3D intolerance characteristics for 102 proteins that included known drug targets with a bound ligand and proteins with known allosteric sites. The corresponding proteins carried a median number of one unique non-overlapping intolerant 3D-site (range 0-6). Overall, 18 proteins lacked an intolerant site, while 32 had greater than one unique intolerant site. Active sites were most constrained, followed by allosteric and ligand binding pockets as shown in FIG. 9A. The lower scores of allosteric sites is consistent with the existing knowledge indicating that these sites tend to be under lower evolutionary conservation pressure than their orthosteric counterparts. We also observed an unequal distribution of tolerant and intolerant binding sites across therapeutic classes as shown in FIG. 9B.

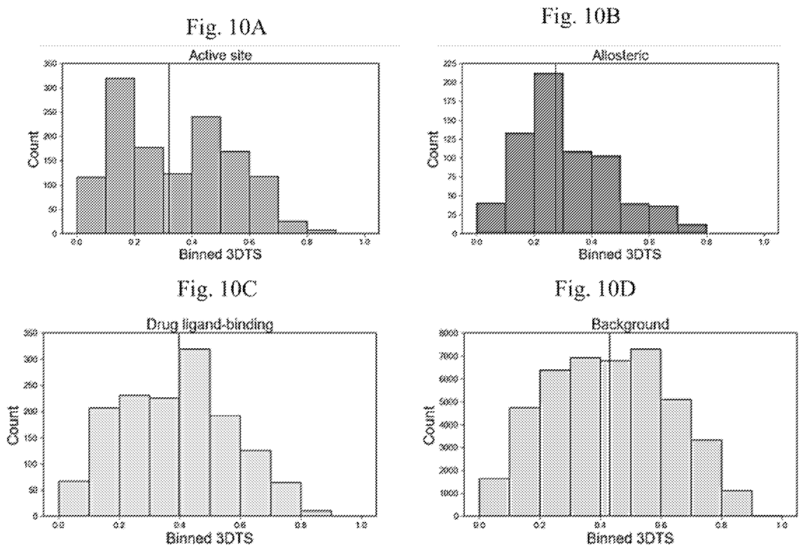

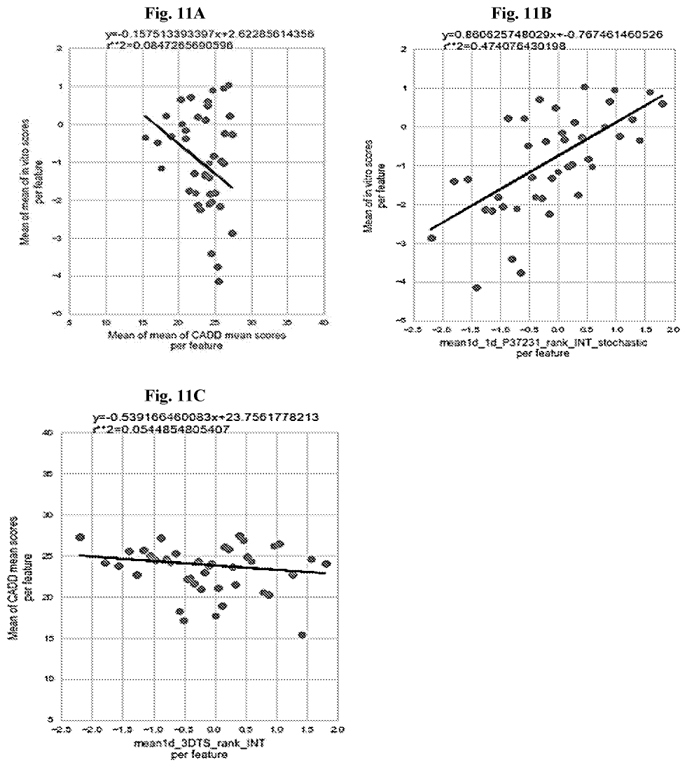

FIG. 9A shows binned 3DTS scores describing active sites, allosteric sites, drug ligand-binding sites, and background. The sum of each site type is 1. Binned counts are provided in FIG. 10A-10D. FIG. 9B shows counts of tolerant and intolerant drug ligand-binding sites grouped by therapeutic area. Here, tolerant is defined as 3DTS>0.5; about 50^{th} percentile score), while intolerant is defined as described in the main text (3DTS<0.33; about 20^{th} percentile score); drug binding sites between these 3DTS values are not included in (b). For example, antineoplastic and immunomodulating agents preferentially target intolerant sites. The identification of multiple intolerant 3D-sites and domains in many drug targets could be exploited for rational drug design and for analysis of drug screening results.

A comprehensive list of druggable protein targets and agents that target such proteins are provided in Table 2 and include, but are not limited to, e.g., the following proteins or binding pockets therein (comprising, e.g., active sites, inhibitory sites, allosteric sites, epitopes), CDK6; DHFR; VDR; SERPINC1; PYGL; MTOR; SRC; FBP1; AMD1; DPEP1; DHFR; MAPK14; IMPDH2; BCHE; DCK; ME2; KIF11; MME; ITGAL; MAOB; MAOB; MAP2K2; MAP2K1; CASP7; PTPN1; PKM; BRAF; GCK; PYGM; DPP4; PDK2; ALB; MAOA; HBA2; HBB; XDH; CA1; CASP1; EGFR; PRPS1; PANK3; APEX1; NT5C2; TYMS; AR; FKBP1A; PKLR; HDAC4; CDK4; MAOB; PDE10A; PDE5A; C5; RXRA; PPARG; MAP2K1; ITGA2B; ITGB3; CA6; CHKB; LTA4H; CA4; TYMS; ABL2; CSNK2A1; PDPK1; PDE4D; ADA; ITGAV; ITGB3; MIF; CHEK1; REN; CA2; SERPINC1; TTR; TTR; CA7; FDPS; MAPK8; UGDH; CDK2; DDC; CDC34; CYP19A1; GLS; CA3; DHODH; HDAC3; HDAC1; PLG; PRMT3; ACHE; CCR5; CHRM2; FDPS; COMT; PDE4B; PDE9A; AGTR1; CA14; HDAC8; PIK3CD; F2; PTGS2; CRBN; CSNK1A1; and SLC6A4. The complete names/sequences of these proteins, including variants thereof, can be obtained from UNIPROT database.

TABLE 2

| | | | | Therapeutic | | | | | | | | | |
|---|---|---|---|---|---|---|---|---|---|---|---|---|---|
| | | | | Drug | | | | | | | | | |
| | | | | Liganded: | | | | | | | | | |
| | | | | already | | | | | | | | | |
| | | | | bound or | | | | | Allosteric | | | | Number |
| | | | | PDB | | | | Median of | Site Residues | Median | | | of Non- |
| | | | | superimposed | Therapeutic | | Top level | Therapeutic | Defined? | of | Active | Median | overlapping |
| | | | Gene | (Drug | Drug with | | ATC code | Ligand | (Ligand(s) or No | Allosteric | Site | of Active | Constrained |
| PDB | Chain | Uniprot | Name | molecule)? | ATC code | ATC code | definition | Score | Ligand Present)? | Score | Feature? | Site Score | Sites |
| 1ANX | A | P08758 | ANXA5 | | | | | | Yes (No ligand) | 0.1527145 | no | | 1 |
| 1B2Y | A | P04746 | AMY2A | | | | | | Yes (CHLORIDE | 0.2156537 | yes | 0.183735 | 1 |
| | | | | | | | | | ION) | | | | |
| 1BLX | A | Q00534 | CDK6 | 5L2I | Palbociclib | LO1XE33 | Antineoplastic and | 0.0382124 | no | | yes | 0.0425578 | 1 |
| | | | | (Palbociclib) | | | immunomodulating | | | | | | |
| | | | | | | | agents | | | | | | |
| 1BOZ | A | P00374 | DHFR | | | | | 0.1478278 | Yes (NADPH) | 0.1769424 | no | | 1 |
| 1DB1 | A | P11473 | VDR | | | | | | Yes (5-{2-[1-(5- | 0.0714783 | no | | 1 |
| | | | | | | | | | HYDROXY-1,5- | | | | |
| | | | | | | | | | DIMETHYL- | | | | |
| | | | | | | | | | HEXYL)-7A- | | | | |
| | | | | | | | | | METHYL- | | | | |
| | | | | | | | | | OCTAHYDRO- | | | | |
| | | | | | | | | | INDEN-4- | | | | |
| | | | | | | | | | YLIDENE]- | | | | |
| | | | | | | | | | ETHYLIDENE}- | | | | |
| | | | | | | | | | 4-METHYLENE- | | | | |
| | | | | | | | | | CYCLOHEXANE- | | | | |
| | | | | | | | | | 1,3-DIOL) | | | | |
| 1E03 | L | P01008 | SERPINC1 | | | | | 0.2004486 | Yes (HEPARIN | 0.2004486 | no | | 3 |
| | | | | | | | | | PENTASACCHARIDE) | | | | |
| 1FA9 | A | Q641R5 | PYGL | | | | | | Yes | 0.3281563 | no | | 0 |
| | | | | | | | | | (ADENOSINE | | | | |
| | | | | | | | | | MONOPHOSPHATE; | | | | |
| | | | | | | | | | ALPHA-D- | | | | |
| | | | | | | | | | GLUCOSE; | | | | |
| | | | | | | | | | PYRIDOXAL-5′- | | | | |
| | | | | | | | | | PHOSPHATE) | | | | |
| 1FAP | B | P42345 | MTOR | | | | | | Yes | 0.0726606 | no | | 1 |
| | | | | | | | | | (RAPAMYCIN) | | | | |
| 1FMK | A | P12931 | SRC | 3QLG | Dasatinib; | L01XE06; | Antineoplastic and | 0.063806 | no | | no | | 1 |
| | | | | (Dasatinib); | Bosutinib | L01XE14 | immunomodulating | | | | | | |
| | | | | 4MXO | | | agents | | | | | | |
| | | | | (Bosutinib) | | | | | | | | | |
| 1FTA | A | P09467 | FBP1 | | | | | | Yes | 0.1146816 | no | | 1 |
| | | | | | | | | | (ADENOSINE | | | | |
| | | | | | | | | | MONOPHOSPHATE) | | | | |
| 1FTA | B | P09467 | FBP1 | | | | | | Yes | 0.0949295 | no | | 1 |
| | | | | | | | | | (ADENOSINE | | | | |
| | | | | | | | | | MONOPHOSPHATE) | | | | |
| 1FTA | C | P09467 | FBP1 | | | | | | Yes | 0.0949295 | no | | 1 |
| | | | | | | | | | (ADENOSINE | | | | |
| | | | | | | | | | MONOPHOSPHATE) | | | | |
| 1FTA | D | P09467 | FBP1 | | | | | | Yes | 0.0907637 | no | | 1 |
| | | | | | | | | | (ADENOSINE | | | | |
| | | | | | | | | | MONOPHOSPHATE) | | | | |
| 1I7M | A | P17707 | AMD1 | | | | | | Yes | 0.0738081 | yes | 0.0738081 | 1 |
| | | | | | | | | | (PUTRESCINE) | | | | |
| 1I7M | B | P17707 | AMD1 | | | | | | Yes | 0.1004425 | yes | 0.0550157 | 1 |
| | | | | | | | | | (PUTRESCINE) | | | | |
| 1I7M | C | P17707 | AMD1 | | | | | | Yes | 0.0832564 | yes | 0.0743469 | 1 |
| | | | | | | | | | (PUTRESCINE) | | | | |
| 1I7M | D | P17707 | AMD1 | | | | | | Yes | 0.0814986 | yes | 0.0588124 | 1 |
| | | | | | | | | | (PUTRESCINE) | | | | |
| 1ITU | A | P16444 | DPEP1 | Already | Cilastatin | **J01 | **Antiinfectives | 0.2998594 | no | | no | | 0 |
| | | | | bound | | | for systemic use | | | | | | |
| | | | | (Cilastatin) | | | | | | | | | |
| 1ITU | B | P16444 | DPEP1 | Already | Cilastatin | **J01 | **Antiinfectives | 0.2998594 | no | | no | | 0 |
| | | | | bound | | | for systemic use | | | | | | |
| | | | | (Cilastatin) | | | | | | | | | |
| 1KMV | A | P00374 | DHFR | 1RG7 | Methotrexate; | L01BA01; | Antineoplastic and | 0.1264216 | no | 0.1986935 | no | | 1 |
| | | | | (Methotrexate); | Pemetrexed | L04AX03; | immunomodulating | | | | | | |
| | | | | 3KSH | | L01BA04 | agents | | | | | | |
| | | | | (Pemetrexed) | | | | | | | | | |
| 1KV1 | A | Q8TDX0 | MAPK14 | | | | | | Yes (1-(5-TERT- | 0.0857089 | yes | 0.0220813 | 1 |
| | | | | | | | | | BUTYL-2- | | | | |
| | | | | | | | | | METHYL-2H- | | | | |
| | | | | | | | | | PYRAZOL-3-YL)- | | | | |
| | | | | | | | | | 3-(4-CHLORO- | | | | |
| | | | | | | | | | PHENYL)-UREA) | | | | |
| 1NF7 | A | P12268 | IMPDH2 | Already | Mycophenolic | L04AA06 | Antineoplastic and | 0.1418961 | no | | yes | 0.1736127 | 1 |
| | | | | bound (C2- | acid | | immunomodulating | | | | | | |
| | | | | MYCOPHENOLIC | | | agents | | | | | | |
| | | | | ADENINE | | | | | | | | | |
| | | | | DINUCLEOTIDE) | | | | | | | | | |
| 1P0I | A | P06276 | BCHE | 4BDS | Tacrine; | N06DA01; | Nervous system | 0.2746921 | no | | yes | 0.2532988 | 0 |
| | | | | (Tacrine); | Rivastigmine | N06DA03 | | | | | | | |
| | | | | 1GQR | | | | | | | | | |
| | | | | (Rivastigmine) | | | | | | | | | |
| 1P5Z | B | P27707 | DCK | 2A7Q | Clofarabine | L01BB06 | Antineoplastic and | 0.1375923 | no | | yes | 0.1304815 | 1 |
| | | | | (Clofarabine) | | | immunomodulating | | | | | | |
| | | | | | | | agents | | | | | | |
| 1PJ2 | A | P23368 | ME2 | | | | | | Yes (FUMARIC ACID; | 0.1467049 | yes | 0.2050891 | 0 |
| | | | | | | | | | MALATE ION; 1,4- | | | | |
| | | | | | | | | | DIHYDRONICOTINAMIDE | | | | |
| | | | | | | | | | ADENINE | | | | |
| | | | | | | | | | DINUCLEOTIDE) | | | | |
| 1PJ2 | B | P23368 | ME2 | | | | | | Yes (FUMARIC ACID; | 0.1467049 | yes | 0.2098848 | 0 |
| | | | | | | | | | MALATE ION; 1,4- | | | | |
| | | | | | | | | | DIHYDRONICOTINAMIDE | | | | |
| | | | | | | | | | ADENINE | | | | |
| | | | | | | | | | DINUCLEOTIDE) | | | | |
| 1PJ2 | C | P23368 | ME2 | | | | | | Yes (FUMARIC ACID; | 0.1467049 | yes | 0.2131642 | 0 |
| | | | | | | | | | MALATE ION; 1,4- | | | | |
| | | | | | | | | | DIHYDRONICOTINAMIDE | | | | |
| | | | | | | | | | ADENINE | | | | |
| | | | | | | | | | DINUCLEOTIDE) | | | | |
| 1PJ2 | D | P23368 | ME2 | | | | | | Yes (FUMARIC ACID; | | | | |
| | | | | | | | | | MALATE ION; 1,4- | | | | |
| | | | | | | | | | DIHYDRONICOTINAMIDE | | | | |
| | | | | | | | | | ADENINE | | | | |
| | | | | | | | | | DINUCLEOTIDE) | 0.1998808 | yes | 0.2066633 | 0 |
| 1Q0B | A | P52732 | KIF11 | | | | | | Yes | 0.1027383 | no | | 1 |
| | | | | | | | | | (MONASTROL) | | | | |
| 1R1H | A | P08473 | MME | 5JMY | Sacubitril | C09DX04 | Cardiovascular | 0.1904954 | no | | yes | 0.1241419 | 2 |
| | | | | (Sacubitril | | | system | | | | | | |
| | | | | active | | | | | | | | | |
| | | | | metabolite) | | | | | | | | | |
| 1RD4 | A | P20701 | ITGAL | | | | | | Yes (1-ACETYL- | 0.118884 | no | | 1 |
| | | | | | | | | | 4-(4-{4-[(2- | | | | |
| | | | | | | | | | ETHOXYPHENYL)THIO]- | | | | |
| | | | | | | | | | 3-NITROPHENYL} | | | | |
| | | | | | | | | | PYRIDIN-2- | | | | |
| | | | | | | | | | YL)PIPERAZINE) | | | | |
| 1S3E | A | P27338 | MAOB | Already | Rasagiline | N04BD02 | Nervous system | | no | | no | | 0 |
| | | | | bound (5- | | | | | | | | | |
| | | | | HYDROXY- | | | | | | | | | |
| | | | | N- | | | | | | | | | |
| | | | | PROPARGYL- | | | | | | | | | |
| | | | | 1(R)- | | | | | | | | | |
| | | | | AMINOINDAN) | | | | | | | | | |
| 1S3E | B | P27338 | MAOB | Already | Rasagiline | N04BD02 | Nervous system | | no | | no | | 0 |
| | | | | bound (5- | | | | | | | | | |
| | | | | HYDROXY- | | | | | | | | | |
| | | | | N- | | | | | | | | | |
| | | | | PROPARGYL- | | | | | | | | | |
| | | | | 1(R)- | | | | | | | | | |
| | | | | AMINOINDAN) | | | | | | | | | |
| 1S91 | A | P36507 | MAP2K2 | Already | Trametinib | L01XE25 | Antineoplastic and | 0.1635953 | Yes (5-{3,4- | 0.1631411 | yes | 0.0946315 | 2 |
| | | | | bound (5- | | | immunomodulating | | DIFLUORO-2-[(2- | | | | |
| | | | | {3,4- | | | agents | | FLUORO-4- | | | | |
| | | | | DIFLUORO- | | | | | IODOPHENYL) | | | | |
| | | | | 2-[(2- | | | | | AMINO]PHENYL}- | | | | |
| | | | | FLUORO-4- | | | | | N-(2- | | | | |
| | | | | IODOPHENYL) | | | | | MORPHOLIN-4- | | | | |
| | | | | AMINO]PHENYL}- | | | | | YLETHYL)-1,3,4- | | | | |
| | | | | N-(2- | | | | | OXADIAZOL-2- | | | | |
| | | | | MORPHOLIN-4- | | | | | AMINE; | | | | |
| | | | | YLETHYL)- | | | | | ADENOSINE-5′- | | | | |
| | | | | 1,3,4- | | | | | TRIPHOSPHATE) | | | | |
| | | | | OXADIAZOL- | | | | | | | | | |
| | | | | 2-AMINE) | | | | | | | | | |
| 1S91 | B | P36507 | MAP2K2 | Already | Trametinib | L01XE25 | Antineoplastic and | 0.1730667 | Yes (5-{3,4- | 0.1730667 | yes | 0.0946315 | 2 |
| | | | | bound (5- | | | immunomodulating | | DIFLUORO-2-[(2- | | | | |
| | | | | {3,4- | | | agents | | FLUORO-4- | | | | |
| | | | | DIFLUORO- | | | | | IODOPHENYL) | | | | |
| | | | | 2-[(2- | | | | | AMINO]PHENYL}- | | | | |
| | | | | FLUORO-4- | | | | | N-(2- | | | | |
| | | | | IODOPHENYL) | | | | | MORPHOLIN-4- | | | | |
| | | | | AMINO]PHENYL}- | | | | | YLETHYL)-1,3,4- | | | | |
| | | | | N-(2- | | | | | OXADIAZOL-2- | | | | |
| | | | | MORPHOLIN-4- | | | | | AMINE; | | | | |
| | | | | YLETHYL)- | | | | | ADENOSINE-5′- | | | | |
| | | | | 1,3,4- | | | | | TRIPHOSPHATE) | | | | |
| | | | | OXADIAZOL-2- | | | | | | | | | |
| | | | | AMINE) | | | | | | | | | |
| 1S9J | A | Q02750 | MAP2K1 | | | | | 0.018852 | Yes | 0.018852 | yes | 0.0392114 | 1 |
| | | | | | | | | | (ADENOSINE-5′- | | | | |
| | | | | | | | | | TRIPHOSPHATE; | | | | |
| | | | | | | | | | 5-BROMO-N- | | | | |
| | | | | | | | | | (2,3- | | | | |
| | | | | | | | | | DIHYDROXYPROPDXY)- | | | | |
| | | | | | | | | | 3,4-DIFLUORO-2- | | | | |
| | | | | | | | | | [(2-FLUORO-4- | | | | |
| | | | | | | | | | IODOPHENYL) | | | | |
| | | | | | | | | | AMINOMENZAMIDE) | | | | |
| 1SHJ | A | Q96BA0 | CASP7 | | | | | | Yes (DICA) | 0.2400717 | yes | 0.2154554 | 0 |
| 1SHJ | B | Q96BA0 | CASP7 | | | | | | Yes (DICA) | 0.2639644 | yes | 0.2115251 | 0 |
| 1T48 | A | P18031 | PTPN1 | | | | | | Yes (3-(3,5- | 0.0678376 | yes | 0.0751864 | 1 |
| | | | | | | | | | DIBROMO-4- | | | | |
| | | | | | | | | | HYDROXY- | | | | |
| | | | | | | | | | BENZOYL)-2- | | | | |
| | | | | | | | | | ETHYL- | | | | |
| | | | | | | | | | BENZOFURAN- | | | | |
| | | | | | | | | | 6-SULFONIC | | | | |
| | | | | | | | | | ACID | | | | |
| | | | | | | | | | DIMETHYLAMIDE) | | | | |
| 1T5A | A | Q96E76 | PKM | | | | | | Yes (BETA- | 0.1973273 | no | | 1 |
| | | | | | | | | | FRUCTOSE-1,6- | | | | |
| | | | | | | | | | DIPHOSPHATE; | | | | |
| | | | | | | | | | OXALATE ION) | | | | |
| 1T5A | B | Q96E76 | PKM | | | | | | Yes (BETA- | 0.1973273 | no | | 1 |
| | | | | | | | | | FRUCTOSE-1,6- | | | | |
| | | | | | | | | | DIPHOSPHATE; | | | | |
| | | | | | | | | | OXALATE ION) | | | | |
| 1T5A | C | Q96E76 | PKM | | | | | | Yes (BETA- | 0.194374 | no | | 1 |
| | | | | | | | | | FRUCTOSE-1,6- | | | | |
| | | | | | | | | | DIPHOSPHATE; | | | | |
| | | | | | | | | | OXALATE ION) | | | | |
| 1T5A | D | Q96E76 | PKM | | | | | | Yes (BETA- | 0.1973273 | no | | 1 |
| | | | | | | | | | FRUCTOSE-1,6- | | | | |
| | | | | | | | | | DIPHOSPHATE; | | | | |
| | | | | | | | | | OXALATE ION) | | | | |
| 1UWH | A | Q13878 | BRAF | | | | | | Yes (4-{4-[({[4- | 0.0964584 | yes | 0.0457447 | 1 |
| | | | | | | | | | CHLORO-3- | | | | |
| | | | | | | | | | (TRIFLUOROMETHYL) | | | | |
| | | | | | | | | | PHENYL] | | | | |
| | | | | | | | | | AMINO}CARBONYL) | | | | |
| | | | | | | | | | AMINO]PHENOXY}-N- | | | | |
| | | | | | | | | | METHYLPYRIDINE- | | | | |
| | | | | | | | | | 2-CARBOXAMIDE) | | | | |
| 1UWH | B | Q13878 | BRAF | | | | | | Yes (4-{4-[({[4- | 0.0804753 | yes | 0.0503783 | 1 |
| | | | | | | | | | CHLORO-3- | | | | |
| | | | | | | | | | (TRIFLUOROME | | | | |
| | | | | | | | | | THYL)PHENYL] | | | | |
| | | | | | | | | | AMINO}CARBONYL) | | | | |
| | | | | | | | | | AMINO]PHENOXY}-N- | | | | |
| | | | | | | | | | METHYLPYRIDINE-2- | | | | |
| | | | | | | | | | CARBOXAMIDE) | | | | |
| 1V45 | A | P35557 | GCK | | | | | | Yes (ALPHA-D- | 0.1125728 | no | | 1 |
| | | | | | | | | | GLUCOSE; 2- | | | | |
| | | | | | | | | | AMINO-4- | | | | |
| | | | | | | | | | FLUORO-5-[(1- | | | | |
| | | | | | | | | | METHYL-1H- | | | | |
| | | | | | | | | | IMIDAZOL-2- | | | | |
| | | | | | | | | | YL)SULFANYL]- | | | | |
| | | | | | | | | | N-(1,3-THIAZOL-2- | | | | |
| | | | | | | | | | YL)BENZAMIDE) | | | | |
| 1Z8D | A | P11217 | PYGM | | | | | | Yes (ADENINE; | 0.3747848 | no | | 0 |
| | | | | | | | | | ADENOSINE | | | | |
| | | | | | | | | | MONOPHOSPHATE; | | | | |
| | | | | | | | | | ALPHA-D- | | | | |
| | | | | | | | | | GLUCOSE) | | | | |
| 2BGR | A | P27487 | DPP4 | 3G0B | Alogliptin | A10BH04; | Alimentary | 0.1700525 | no | | yes | 0.1700525 | 2 |
| | | | | (Alogliptin); | Linagliptin; | A10BH05; | tract and | | | | | | |
| | | | | 2RGU | Saxagliptin; | A10BH03; | metabolism | | | | | | |
| | | | | (Linagliptin); | Sitagliptin | A10BH01 | | | | | | | |
| | | | | 3BJM | | | | | | | | | |
| | | | | (Saxagliptin); | | | | | | | | | |
| | | | | 1X70 | | | | | | | | | |
| | | | | (Sitagliptin) | | | | | | | | | |
| 2BGR | B | P27487 | DPP4 | 3G0B | Alogliptin; | A10BH04; | Alimentary | 0.1701216 | no | | yes | 0.1785257 | 2 |
| | | | | (Alogliptin); | Linagliptin; | A10BH05; | tract and | | | | | | |
| | | | | 2RGU | Saxagliptin; | A10BH03; | metabolism | | | | | | |
| | | | | (Linagliptin); | Sitagliptin | A10BH01 | | | | | | | |
| | | | | 3BJM | | | | | | | | | |
| | | | | (Saxagliptin); | | | | | | | | | |
| | | | | 1X70 | | | | | | | | | |
| | | | | (Sitagliptin) | | | | | | | | | |
| 2BU2 | A | Q15119 | PDK2 | | | | | | Yes | 0.147293 | no | | 4 |
| | | | | | | | | | (ADENOSINE-5′- | | | | |
| | | | | | | | | | TRIPHOSPHATE; | | | | |
| | | | | | | | | | 4-({(2R,5S)-2,5- | | | | |
| | | | | | | | | | DIMETHYL-4- | | | | |
| | | | | | | | | | [(2R)-3,3,3- | | | | |
| | | | | | | | | | TRIFLUORO-2- | | | | |
| | | | | | | | | | HYDROXY-2- | | | | |
| | | | | | | | | | METHYLPROPANOYL] | | | | |
| | | | | | | | | | PIPERAZIN-1- | | | | |
| | | | | | | | | | YL}CARBONYL) | | | | |
| | | | | | | | | | BENZONITRILE) | | | | |
| 2BXD | A | Q86YG0 | ALB | | | | | | Yes (R- | 0.1516153 | no | | 5 |
| | | | | | | | | | WARFARIN) | | | | |
| 2BXR | A | P21397 | MAOA | Already | Rasagiline | N04BD02 | Nervous system | | no | | no | | 0 |
| | | | | bound (N-[3- | | | | | | | | | |
| | | | | (2,4- | | | | | | | | | |
| | | | | DICHLORO | | | | | | | | | |
| | | | | PHENOXY) | | | | | | | | | |
| | | | | PROPYL]- | | | | | | | | | |
| | | | | N- | | | | | | | | | |
| | | | | METHYL- | | | | | | | | | |
| | | | | N-PROP-2- | | | | | | | | | |
| | | | | YNYLAMINEN- | | | | | | | | | |
| | | | | METHYL-N- | | | | | | | | | |
| | | | | PROPARGYL- | | | | | | | | | |
| | | | | 3-(2,4- | | | | | | | | | |
| | | | | DICHLORO | | | | | | | | | |
| | | | | PHENOXY) | | | | | | | | | |
| | | | | PROPYLAMINE) | | | | | | | | | |
| 2D5Z | A | P69905 | HBA2 | | | | | | Yes | 0.1655126 | no | | 1 |
| | | | | | | | | | (PROTOPORPHYRIN | | | | |
| | | | | | | | | | IX CONTAINING | | | | |
| | | | | | | | | | FE; 2-[4-({[(3,5- | | | | |
| | | | | | | | | | DICHLOROPHENYL) | | | | |
| | | | | | | | | | AMINO]CARBONYL} | | | | |
| | | | | | | | | | AMINO)PHENOXY]-2- | | | | |
| | | | | | | | | | METHYLPROPANOIC | | | | |
| | | | | | | | | | ACID) | | | | |
| 2D5Z | B | Q549N7 | HBB | | | | | | Yes | 0.2098042 | no | | 1 |
| | | | | | | | | | (PROTOPORPHYRIN IX | | | | |
| | | | | | | | | | CONTAINING | | | | |
| | | | | | | | | | FE; 2-[4-({[(3,5- | | | | |
| | | | | | | | | | DICHLOROPHENYL) | | | | |
| | | | | | | | | | AMINO]CARBONYL} | | | | |
| | | | | | | | | | AMINO)PHENOXY]-2- | | | | |
| | | | | | | | | | METHYLPROPANOIC | | | | |
| | | | | | | | | | ACID) | | | | |
| 2D5Z | C | P69905 | HBA2 | | | | | | Yes | 0.1655126 | no | | 1 |
| | | | | | | | | | (PROTOPORPHYRIN IX | | | | |
| | | | | | | | | | CONTAINING | | | | |
| | | | | | | | | | FE; 2-[4-({[(3,5- | | | | |
| | | | | | | | | | DICHLOROPHENYL) | | | | |
| | | | | | | | | | AMINO]CARBONYL} | | | | |
| | | | | | | | | | AMINO)PHENOXY]-2- | | | | |
| | | | | | | | | | METHYLPROPANOIC | | | | |
| | | | | | | | | | ACID) | | | | |
| 2D5Z | D | Q549N7 | HBB | | | | | | Yes | 0.2173427 | no | | 1 |
| | | | | | | | | | (PROTOPORPHYRIN IX | | | | |
| | | | | | | | | | CONTAINING | | | | |
| | | | | | | | | | FE; 2-[4-({[(3,5- | | | | |
| | | | | | | | | | DICHLOROPHENYL) | | | | |
| | | | | | | | | | AMINO]CARBONYL} | | | | |
| | | | | | | | | | AMINO)PHENOXY]-2- | | | | |
| | | | | | | | | | METHYLPROPANOIC | | | | |
| | | | | | | | | | ACID) | | | | |
| 2E1Q | A | P47989 | XDH | 3BDJ | Allopurinol | M04AA01; | Musculoskeletal | 0.2329392 | no | | yes | 0.2329392 | 0 |
| | | | | (Allopurinol) | | M04AA51 | system | | | | | | |
| 2E1Q | B | P47989 | XDH | 3BDJ | Allopurinol | M04AA01; | Musculoskeletal | 0.2403869 | no | | yes | 0.2609582 | 0 |
| | | | | (Allopurinol) | | M04AA51 | system | | | | | | |
| 2FOY | A | P00915 | CA1 | 3W6H | Acetazolamide; | S01EC01; | Sensory | 0.2369737 | no | | yes | 0.2308594 | 0 |
| | | | | (Acetazolamide); | Methazolamide; | S01EC05; | organs; | | | | | | |
| | | | | 1BZM | Dichlorphenamide; | S01EC02; | *Cardiovascular | | | | | | |
| | | | | (Methazolamide); | Ethoxzolamide | *C03; | system; | | | | | | |
| | | | | 2POU | | *N03 | *Nervous | | | | | | |
| | | | | (Dichlorphenamide); | | | system | | | | | | |
| | | | | 3DD0 | | | | | | | | | |
| | | | | (Ethoxzolamide) | | | | | | | | | |
| 2FQQ | A | P29466 | CASP1 | | | | | | Yes (1-METHYL-3- | 0.2776623 | yes | 0.2578808 | 0 |
| | | | | | | | | | TRIFLUOROMETHYL- | | | | |
| | | | | | | | | | 1H- | | | | |
| | | | | | | | | | THIENO[2,3- | | | | |
| | | | | | | | | | C]PYRAZOLE-5- | | | | |
| | | | | | | | | | CARBOXYLIC | | | | |
| | | | | | | | | | ACID (2- | | | | |
| | | | | | | | | | MERCAPTO- | | | | |
| | | | | | | | | | ETHYL)-AMIDE) | | | | |
| 2FQQ | B | P29466 | CASP1 | | | | | | Yes (1-METHYL-3- | 0.2299838 | yes | 0.2299838 | 0 |
| | | | | | | | | | TRIFLUOROMETHYL- | | | | |
| | | | | | | | | | 1H-THIENO[2,3- | | | | |
| | | | | | | | | | C]PYRAZOLE-5- | | | | |
| | | | | | | | | | CARBOXYLIC | | | | |
| | | | | | | | | | ACID (2- | | | | |
| | | | | | | | | | MERCAPTO- | | | | |
| | | | | | | | | | ETHYL)-AMIDE) | | | | |
| 2GS7 | A | Q9GZX1 | EGFR | | | | | | Yes | 0.2034455 | yes | 0.2165181 | 2 |
| | | | | | | | | | (PHOSPHOAMINOPHOSPHONIC | | | | |
| | | | | | | | | | ACID-ADENYLATE ESTER) | | | | |
| 2GS7 | B | Q9GZX1 | EGFR | | | | | | Yes | 0.2080026 | yes | 0.2037397 | 2 |
| | | | | | | | | | (PHOSPHOAMINOPHOSPHONIC | | | | |
| | | | | | | | | | ACID-ADENYLATE ESTER) | | | | |
| 2H06 | A | P60891 | PRPS1 | | | | | | Yes (SULFATE | | no | | 0 |
| | | | | | | | | | ION) | | | | |
| 2H06 | B | P60891 | PRPS1 | | | | | | Yes (SULFATE | | no | | 0 |
| | | | | | | | | | ION) | | | | |
| 2I7P | A | Q9H999 | PANK3 | | | | | | Yes (ACETYL | 0.1100955 | no | | 1 |
| | | | | | | | | | COENZYME *A) | | | | |
| 2I7P | C | Q9H999 | PANK3 | | | | | | Yes (ACETYL | 0.1100955 | no | | 1 |
| | | | | | | | | | COENZYME *A) | | | | |
| | | | | | | | | | Yes | 0.0529443 | yes | 0.0421036 | 1 |
| 2ISI | A | P27695 | APEX1 | | | | | | (MAGNESIUM | | | | |
| | | | | | | | | | ION) | 0.1184393 | yes | 0.0498008 | 1 |
| 2JC9 | A | P49902 | NT5C2 | | | | | | Yes | | | | |
| | | | | | | | | | (ADENOSINE) | | | | |
| 2ONB | A | P04818 | TYMS | | | | | 0.0785948 | Yes (PROPANE-1,3- | 0.0694786 | yes | 0.0977268 | 1 |
| | | | | | | | | | DIYLBIS(PHOSPHONIC | | | | |
| | | | | | | | | | ACID); | | | | |
| | | | | | | | | | 1,2-ETHANEDIOL) | | | | |
| 2PIT | A | P10275 | AR | | | | | | Yes ([4-(4- | | no | | 0 |
| | | | | | | | | | HYDROXY-3- | | | | |
| | | | | | | | | | IODO- | | | | |
| | | | | | | | | | PHENOXY)-3,5- | | | | |
| | | | | | | | | | DIIODO- | | | | |
| | | | | | | | | | PHENYL]- | | | | |
| | | | | | | | | | ACETIC ACID) | | | | |
| 2PPN | A | P62942 | FKBP1A | 2VCD | Sirolimus | L04AA10 | Antineoplastic and | 0.0765502 | no | | no | | 1 |
| | | | | (Sirolimus) | | | immunomodulating | | | | | | |
| | | | | | | | agents | | | | | | |
| 2VGI | A | P30613 | PKLR | | | | | | Yes (BETA- | 0.2225841 | no | | 2 |
| | | | | | | | | | FRUCTOSE-1,6- | | | | |
| | | | | | | | | | DIPHOSPHATE; | | | | |
| | | | | | | | | | POTASSIUM ION; | | | | |
| | | | | | | | | | MANGANESE | | | | |
| | | | | | | | | | (II) ION; 2- | | | | |
| | | | | | | | | | PHOSPHOGLYCOLIC | | | | |
| | | | | | | | | | ACID) | | | | |
| 2VGI | B | P30613 | PKLR | | | | | | Yes (BETA- | 0.2225841 | no | | 3 |
| | | | | | | | | | FRUCTOSE-1,6- | | | | |
| | | | | | | | | | DIPHOSPHATE; | | | | |
| | | | | | | | | | POTASSIUM ION; | | | | |
| | | | | | | | | | MANGANESE | | | | |
| | | | | | | | | | (II) ION; 2- | | | | |
| | | | | | | | | | PHOSPHOGLYCOLIC | | | | |
| | | | | | | | | | ACID) | | | | |
| 2VGI | C | P30613 | PKLR | | | | | | Yes (BETA- | 0.2225841 | no | | 1 |
| | | | | | | | | | FRUCTOSE-1,6- | | | | |
| | | | | | | | | | DIPHOSPHATE; | | | | |
| | | | | | | | | | POTASSIUM ION; | | | | |
| | | | | | | | | | MANGANESE | | | | |
| | | | | | | | | | (II) ION; 2- | | | | |
| | | | | | | | | | PHOSPHOGLYCOLIC | | | | |
| | | | | | | | | | ACID) | | | | |
| 2VGI | D | P30613 | PKLR | | | | | | Yes (BETA- | 0.2225841 | no | | 3 |
| | | | | | | | | | FRUCTOSE-1,6- | | | | |
| | | | | | | | | | DIPHOSPHATE; | | | | |
| | | | | | | | | | POTASSIUM ION; | | | | |
| | | | | | | | | | MANGANESE | | | | |
| | | | | | | | | | (II) ION; 2- | | | | |
| | | | | | | | | | PHOSPHOGLYCOLIC | | | | |
| | | | | | | | | | ACID) | | | | |
| 2VQJ | A | P56524 | HDAC4 | | | | | | Yes (No ligand) | 0.0399164 | yes | 0.0247204 | 2 |
| 2W96 | B | P11802 | CDK4 | 5L2I | Palbociclib | L01XE33 | Antineoplastic and | 0.1103339 | no | | yes | 0.1103339 | 2 |
| | | | | (Palbociclib) | | | immunomodulating | | | | | | |
| | | | | | | | agents | | | | | | |
| 2XCG | A | P27338 | MAOB | | | | | | Yes (2-(2- | | no | | 0 |
| | | | | | | | | | BENZOFURANYL)- | | | | |
| | | | | | | | | | 2-IMIDAZOLINE) | | | | |
| 2XCG | B | P27338 | MAOB | | | | | | Yes (2-(2- | | no | | 0 |
| | | | | | | | | | BENZOFURANYL)- | | | | |
| | | | | | | | | | 2-IMIDAZOLINE) | | | | |
| 2ZMF | A | Q9Y233 | PDE10A | | | | | | Yes (CYCLIC | 0.0671531 | no | | 1 |
| | | | | | | | | | AMP) | | | | |
| 2ZMF | B | Q9Y233 | PDE10A | | | | | | Yes (CYCLIC | 0.0757655 | no | | 1 |
| | | | | | | | | | AMP) | | | | |
| 3BJC | A | O76074 | PDE5A | 3TVX | Pentoxifylli | C04AD03; | Cardiovascular | 0.0929191 | no | | yes | 0.1106072 | 1 |
| | | | | (Pentoxifylline); | ne; | G04BE03; | system; | | | | | | |
| | | | | 1TBF | Sildenafil; | G04BE08; | Genitourinary | | | | | | |
| | | | | (Sildenafil); | Tadalafil; | G04BE09 | system | | | | | | |
| | | | | 1XOZ | Vardenafil | | and sex | | | | | | |
| | | | | (Tadalafil); | | | hormones | | | | | | |
| | | | | 1XPO | | | | | | | | | |
| | | | | (Vardenafil) | | | | | | | | | |
| 3CU7 | A | P01031 | C5 | 5I5K | Eculizumab | L04AA25 | Antineoplastic and | 0.1756369 | no | | no | | 7 |
| | | | | (Eculizumab) | | | immunomodulating | | | | | | |
| | | | | | | | agents | | | | | | |
| 3DZY | A | P19793 | RXRA | 4K6I | Bexarotene | L01XX25 | Antineoplastic and | 0.0955431 | no | | no | | 1 |
| | | | | (Bexarotene) | | | immunomodulating | | | | | | |
| | | | | | | | agents | | | | | | |
| 3DZY | D | Q15179 | PPARG | Already | Rosiglitazone; | A10BG02; | Alimentary | 0.0945114 | no | | no | | 2 |
| | | | | bound | Pioglitazone | A10BG03 | tract and | | | | | | |
| | | | | (Rosiglitazone); | | | metabolism | | | | | | |
| | | | | 2XKW | | | | | | | | | |
| | | | | (Pioglitazone) | | | | | | | | | |
| 3EQC | A | Q02750 | MAP2K1 | 1S9I (5-{3,4- | Trametinib | L01XE25 | Antineoplastic and | 0.0206118 | no | 0.0206118 | yes | 0.0373669 | 1 |
| | | | | DIFLUORO- | | | immunomodulating | | | | | | |
| | | | | 2-[(2- | | | agents | | | | | | |
| | | | | FLUORO-4- | | | | | | | | | |
| | | | | IODOPHENYL) | | | | | | | | | |
| | | | | AMINO] | | | | | | | | | |
| | | | | PHENYL}- | | | | | | | | | |
| | | | | N-(2- | | | | | | | | | |
| | | | | MORPHOLIN-4- | | | | | | | | | |
| | | | | YLETHYL)- | | | | | | | | | |
| | | | | 1,3,4- | | | | | | | | | |
| | | | | OXADIAZOL-2- | | | | | | | | | |
| | | | | AMINE) | | | | | | | | | |
| 3FCS | A | P08514 | ITGA2B | 2VDN | Eptifibatide; | B01AC16; | Blood and | 0.254818 | no | | no | | 5 |
| | | | | (Eptifibatide); | Tirofiban | B01AC17 | blood | | | | | | |
| | | | | 2VDM | | | forming | | | | | | |
| | | | | (Tirofiban) | | | organs | | | | | | |
| 3FCS | B | P05106 | ITGB3 | 2VDN | Eptifibatide; | | Blood and | 0.20457 | no | | no | | 5 |
| | | | | (Eptifibatide); | Tirofiban | B01AC16; | blood | | | | | | |
| | | | | 2VDM | | B01AC17 | forming | | | | | | |
| | | | | (Tirofiban) | | | organs | | | | | | |
| 3FE4 | A | P23280 | CA6 | 3DD0 | Ethoxzolamide | *S01; | *Sensory | 0.2446723 | no | | yes | 0.2411164 | 1 |
| | | | | (Ethoxzolamide) | | *C03; | organs; | | | | | | |
| | | | | | | *N03 | *Cardiovascular | | | | | | |
| | | | | | | | system; | | | | | | |
| | | | | | | | *Nervous | | | | | | |
| | | | | | | | system | | | | | | |
| 3FEG | A | Q9Y259 | CHKB | | | | | | Yes (No ligand) | 0.1920207 | no | | 1 |
| 3FTS | A | P09960 | LTA4H | | | | | | Yes (No ligand) | 0.1408259 | yes | 0.1416021 | 5 |
| 3FW3 | A | P22748 | CA4 | 3W6H | Acetazolamide; | S01EC01; | Sensory | 0.2951668 | no | | yes | 0.3478934 | 1 |
| | | | | (Acetazolamide); | Methazolamide; | S01EC05; | organs; | | | | | | |
| | | | | 1BZM | Dichloiphenamide; | S01EC02; | *Cardiovascular | | | | | | |
| | | | | (Methazolamide); | Ethoxzolamide; | *C03; | system; | | | | | | |
| | | | | 2POU | Topiramate | N03AX11 | Nervous | | | | | | |
| | | | | (Dichloiphenamide); | | | system | | | | | | |
| | | | | 3DD0 | | | | | | | | | |
| | | | | (Ethoxzolamide); | | | | | | | | | |
| | | | | 3HKU | | | | | | | | | |
| | | | | (Topiramate) | | | | | | | | | |
| 3GH0 | A | P04818 | TYMS | 3K2H | Pemetrexed | L01BA04 | Antineoplastic and | 0.137788 | no | 0.068951 | yes | 0.1063269 | 1 |
| | | | | (Pemetrexed) | | | immunomodulating | | | | | | |
| | | | | | | | agents | | | | | | |
| 3GVU | A | Q6NZY6 | ABL2 | | | | | | Yes (IMATINIB) | 0.140812 | yes | 0.0711063 | 1 |
| 3H30 | A | P68400 | CSNK2A1 | | | | | | Yes (5,6-dichloro- | 0.0309288 | yes | 0.0440474 | 1 |
| | | | | | | | | | 1-beta-D- | | | | |
| | | | | | | | | | ribofuranosyl-1H- | | | | |
| | | | | | | | | | benzimidazole) | | | | |
| 3HRF | A | 015530 | PDPK1 | | | | | | Yes ((2Z)-5-(4- | 0.2418028 | yes | 0.0994019 | 1 |
| | | | | | | | | | chlorophenyl)-3- | | | | |
| | | | | | | | | | phenylpent-2-enoic | | | | |
| | | | | | | | | | acid) | | | | |
| 3IAD | A | Q8IVD2 | PDE4D | | | | | | Yes (1-{4-[(2- | 0.0786396 | yes | 0.0579003 | 1 |
| | | | | | | | | | fluoro-6-methoxy- | | | | |
| | | | | | | | | | 3′-nitrobiphenyl- | | | | |
| | | | | | | | | | 3- | | | | |
| | | | | | | | | | yl)methyl]phenyl} | | | | |
| | | | | | | | | | urea) | | | | |
| 3IAR | A | P00813 | ADA | 2PGR | Pentostatin | L01XX08 | Antineoplastic and | 0.2724563 | no | | yes | 0.2391462 | 0 |
| | | | | (Pentostatin) | | | immunomodulating | | | | | | |
| | | | | | | | agents | | | | | | |
| 3IJE | A | Q59EB7 | ITGAV | 2VDN | Eptifibatide; | B01AC16; | Blood and | 0.1042322 | no | | no | | 5 |
| | | | | (Eptifibatide); | Tirofiban | B01AC17 | blood forming | | | | | | |
| | | | | 2VDM | | | organs | | | | | | |
| | | | | (Tirofiban) | | | | | | | | | |
| 3IJE | B | P05106 | ITGB3 | 2VDN | Eptifibatide; | B01AC16; | Blood and | 0.1817787 | no | | no | | 5 |
| | | | | (Eptifibatide); | Tirofiban | B01AC17 | blood forming | | | | | | |
| | | | | 2VDM | | | organs | | | | | | |
| | | | | (Tirofiban) | | | | | | | | | |
| 3IJG | A | P14174 | MIF | | | | | | yes | 0.3258102 | yes | 0.3252289 | 0 |
| 3IJG | B | P14174 | MIF | | | | | | yes | 0.3392566 | yes | 0.3252289 | 0 |
| 3IJG | C | P14174 | MIF | | | | | | Yes (5-alpyridin- | 0.3258102 | yes | 0.3252289 | 0 |
| | | | | | | | | | 3-yl)propan-1-one) | | | | |
| 3JVR | A | O14757 | CHEK1 | | | | | | Yes ((1S)-1-(1H- | 0.1265213 | yes | 0.0479422 | 3 |
| | | | | | | | | | benzimidazol-2- | | | | |
| | | | | | | | | | yl)ethyl (3,4- | | | | |
| | | | | | | | | | dichlorophenyl)carbamate) | | | | |
| 3K1W | A | P00797 | REN | 2V0Z | Aliskiren | C09XA02 | Cardiovascular | 0.1594976 | no | | yes | 0.1506349 | 1 |
| | | | | (Aliskiren) | | | system | | | | | | |
| 3K34 | A | P00918 | CA2 | 4G0C | Acetazolamide; | S01EC01; | Sensory organs; | 0.1119247 | no | | yes | 0.1504358 | 1 |
| | | | | (Acetazolamide); | Methazolamide; | S01EC05; | *Cardiovascular | | | | | | |
| | | | | 5C8I | Dichlorphenamide; | S01EC02; | system; | | | | | | |
| | | | | (Methazolamide); | Ethoxzolamide; | *C03; | Nervous system | | | | | | |
| | | | | 2POU | Topiramate; | N03AX11; | | | | | | | |
| | | | | (Dichlorphenamide); | Brinzolamide; | S01EC04; | | | | | | | |
| | | | | 3DD0 | Dorzolamide | S01EC03 | | | | | | | |
| | | | | (Ethoxzolamide); | | | | | | | | | |
| | | | | 3HKU | | | | | | | | | |
| | | | | (Topiramate); | | | | | | | | | |
| | | | | 4M2R | | | | | | | | | |
| | | | | (Brinzolamide); | | | | | | | | | |
| | | | | 4M2U | | | | | | | | | |
| | | | | (Dorzolamide) | | | | | | | | | |
| 3KCG | I | P01008 | SERPINC1 | Already bound | Heparin calcium; | B01AB01; | Blood and | 0.211499 | no | 0.211499 | yes | | 3 |
| | | | | (Heparin); | Fondaparinux | B01AB51; | blood forming | | | | | | |
| | | | | 3EVJ | | C05BA03; | organs; | | | | | | |
| | | | | (Fondaparinux) | | C05BA53; | Cardiovascular | | | | | | |
| | | | | | | S01XA14; | system; | | | | | | |
| | | | | | | B01AX05 | Sensory organs | | | | | | |
| 3KGT | A | P02766 | TTR | | | | | | Yes | 0.2810746 | no | | 2 |
| | | | | | | | | | (GENISTEOL) | | | | |
| 3KGT | B | P02766 | TTR | | | | | | Yes | 0.2918951 | no | | 2 |
| | | | | | | | | | (GENISTEOL) | | | | |
| 3ML5 | A | P43166 | CA7 | 3MDZ | Ethoxzolamide; | *C03; | Sensory organs; | 0.0612488 | no | | yes | 0.0668757 | 1 |
| | | | | (Ethoxzolamide); | Methazolamide; | *N03; | *Cardiovascular | | | | | | |
| | | | | 1BZM | Dichlorphenamide | S01EC05; | system; | | | | | | |
| | | | | (Methazolamide); | | S01EC02 | *Nervous system | | | | | | |
| | | | | 2P0U | | | | | | | | | |
| | | | | (Dichlorphenamide) | | | | | | | | | |
| 3N45 | F | P14324 | FDPS | | | | | 0.1539435 | Yes ((2S)-1- | 0.1793133 | no | | 2 |
| | | | | | | | | | [(benzyloxy)carbonyl]- | | | | |
| | | | | | | | | | 2,3-dihydro- | | | | |
| | | | | | | | | | 1H-indole-2- | | | | |
| | | | | | | | | | carboxylic acid) | | | | |
| 3O2M | A | Q308M2 | MAPK8 | | | | | | Yes (N-butyl-4,6- | 0.0838781 | yes | 0.0365927 | 1 |
| | | | | | | | | | dimethyl-N-{[2′- | | | | |
| | | | | | | | | | (2H-tetrazol- 5- | | | | |
| | | | | | | | | | yl)biphenyl-4- | | | | |
| | | | | | | | | | yl]methyl}pyrimidin- | | | | |
| | | | | | | | | | 2-amine) | | | | |
| 3PRJ | A | O60701 | UGDH | | | | | | Yes (UDP- | 0.0568902 | yes | 0.0683475 | 1 |
| | | | | | | | | | ALPHA-D- | | | | |
| | | | | | | | | | XYLOPYRANOSE) | | | | |
| 3PRJ | B | O60701 | UGDH | | | | | | Yes (UDP- | 0.0584238 | yes | 0.0683475 | 1 |
| | | | | | | | | | ALPHA-D- | | | | |
| | | | | | | | | | XYLOPYRANOSE) | | | | |
| 3PRJ | C | O60701 | UGDH | | | | | | Yes (UDP- | 0.0568902 | yes | 0.0683475 | 1 |
| | | | | | | | | | ALPHA-D- | | | | |
| | | | | | | | | | XYLOPYRANOSE) | | | | |
| 3PRJ | D | O60701 | UGDH | | | | | | Yes (UDP- | 0.0584238 | yes | 0.0729025 | 1 |
| | | | | | | | | | ALPHA-D- | | | | |
| | | | | | | | | | XYLOPYRANOSE) | | | | |
| 3PRJ | E | O60701 | UGDH | | | | | | Yes (UDP- | 0.056129 | yes | 0.0716465 | 1 |
| | | | | | | | | | ALPHA-D- | | | | |
| | | | | | | | | | XYLOPYRANOSE) | | | | |
| 3PRJ | F | O60701 | UGDH | | | | | | Yes (UDP- | 0.0568902 | yes | 0.0683475 | 1 |
| | | | | | | | | | ALPHA-D- | | | | |
| | | | | | | | | | XYLOPYRANOSE) | | | | |
| 3PXF | A | P24941 | CDK2 | | | | | | Yes (8-ANILINO-1- | 0.078535 | yes | | 1 |
| | | | | | | | | | NAPHTHALENE | | | | |
| | | | | | | | | | SULFONATE) | | | | |
| 3RCH | A | P20711 | DDC | 1JSE | Carbidopa | **N04 | **Nervous | 0.1717718 | no | | no | | 0 |
| | | | | (Carbidopa) | | | system | | | | | | |
| 3RCH | B | P20711 | DDC | 1JSE | Carbidopa | **N04 | **Nervous | 0.1959311 | no | | no | | 1 |
| | | | | (Carbidopa) | | | system | | | | | | |
| 3RZ3 | A | P49427 | CDC34 | | | | | | Yes (4,5-dideoxy- | 0.088017 | yes | 0.1226621 | 1 |
| | | | | | | | | | 5-(3′,5′- | | | | |
| | | | | | | | | | dichlorobiphenyl- | | | | |
| | | | | | | | | | 4-yl)-4- | | | | |
| | | | | | | | | | [(methoxyacetyl)amino]- | | | | |
| | | | | | | | | | L-arabinonic acid) | | | | |
| 3S79 | A | P11511 | CYP19A1 | 3S7S | Exemestane | L02BG06 | Antineoplastic and | 0.245081 | no | 0.1745211 | no | | 1 |
| | | | | (Exemestane) | | | immunomodulating | | | | | | |
| | | | | | | | agents | | | | | | |
| 3S7S | A | P11511 | CYP19A1 | | | | | 0.2358124 | Yes (No ligand) | 0.187625 | no | | 1 |
| 3SWZ | A | P05093 | CYP17A1 | 3RUK | Abiraterone | L02BX03; | Antineoplastic and | 0.2004937 | no | | no | | 1 |
| | | | | (Abiraterone); | acetate; | D01AC08; | immunomodulating | | | | | | |
| | | | | 3LD6 | Ketoconazole | G01AF11; | agents; | | | | | | |
| | | | | (Ketoconazole) | | J02AB02 | Dermatologicals; | | | | | | |
| | | | | | | | Genito- | | | | | | |
| | | | | | | | urinary system | | | | | | |
| | | | | | | | and sex | | | | | | |
| | | | | | | | hormones; | | | | | | |
| | | | | | | | Antiinfectives for | | | | | | |
| | | | | | | | systemic use | | | | | | |
| 3UO9 | A | 094925 | GLS | | | | | | Yes (BPTES) | 0.0393675 | no | | 1 |
| 3UO9 | B | 094925 | GLS | | | | | | Yes (BPTES) | 0.0393675 | no | | 1 |
| 3UO9 | C | 094925 | GLS | | | | | | Yes (BPTES) | 0.0393675 | no | | 1 |
| 3UO9 | D | 094925 | GLS | | | | | | Yes (BPTES) | 0.0391718 | no | | 1 |
| 3UYQ | A | P07451 | CA3 | 3DD0 | Ethoxzolamide | *S01; | *Sensory organs; | 0.2163017 | no | | yes | 0.1994709 | 2 |
| | | | | (Ethoxzolamide) | | *C03; | *Cardiovascular | | | | | | |
| | | | | | | *N03 | system; | | | | | | |
| | | | | | | | *Nervous system | | | | | | |
| 3ZWT | A | Q02127 | DHODH | 3FJ6 | Leflunomide | L04AA13; | Antineoplastic and | 0.2651646 | no | | yes | 0.196239 | 0 |
| | | | | (Leflunomide | | L04AA31 | immunomodulating | | | | | | |
| | | | | derivative) | | | agents | | | | | | |
| 4A69 | A | O15379 | HDAC3 | SEEN | Belinostat; | L01XX49; | Antineoplastic and | 0.0513427 | no | | no | | 1 |
| | | | | (Belinostat); | Panobinostat; | L01XX42; | immunomodulating | | | | | | |
| | | | | 5EF8 | Vorinostat | L01XX38 | agents | | | | | | |
| | | | | (Panobinostat); | | | | | | | | | |
| | | | | 4LXZ | | | | | | | | | |
| | | | | (Vorinostat) | | | | | | | | | |
| 4BKX | B | Q13547 | HDAC1 | SEEN | Belinostat; | L01XX49; | Antineoplastic and | 0.0583657 | no | | no | | 1 |
| | | | | (Belinostat); | Panobinostat; | L01XX42; | immunomodulating | | | | | | |
| | | | | 5EF8 | Vorinostat | L01XX38 | agents | | | | | | |
| | | | | (Panobinostat); | | | | | | | | | |
| | | | | 4LXZ | | | | | | | | | |
| | | | | (Vorinostat) | | | | | | | | | |
| 4DUR | A | P00747 | PLG | 1CEB | Tranexamic | B02AA02; | Blood and | 0.1798332 | no | | no | | 6 |
| | | | | (Tranexamic); | acid; | B02AB01; | blood forming | | | | | | |
| | | | | 3UIR | Aprotinin; | B02AA01; | organs | | | | | | |
| | | | | (Aprotinin | Aminocaproic | B01AD01; | | | | | | | |
| | | | | analog); | acid; | B06AA55 | | | | | | | |
| | | | | 1HPK | Streptokinase | | | | | | | | |
| | | | | (Aminocaproic | | | | | | | | | |
| | | | | acid); | | | | | | | | | |
| | | | | 1BML | | | | | | | | | |
| | | | | (Streptokinase); | | | | | | | | | |
| | | | | 1L4Z | | | | | | | | | |
| | | | | (Steptokinase) | | | | | | | | | |
| 4HSG | A | O60678 | PRMT3 | | | | | | Yes (1-(1,2,3- | 0.2895833 | yes | 0.1806625 | 0 |
| | | | | | | | | | benzothiadiazol-6- | | | | |
| | | | | | | | | | yl)-3-(2-oxo-2- | | | | |
| | | | | | | | | | phenylethyl)urea) | | | | |
| 4M0E | A | Q53F46 | ACHE | 2WG1 (N-hydroxy- | Pralidoxime | V03AB04 | Various | 0.0486244 | no | | yes | 0.0861735 | 1 |
| | | | | 1-(1- | | | | | | | | | |
| | | | | methylpyridin-2(1H)- | | | | | | | | | |
| | | | | ylidene)methanamine) | | | | | | | | | |
| 4MBS | A | O14708 | CCR5 | Already | Maraviroc | J05AX09 | Antiinfectives for | 0.2890593 | no | | no | | 0 |
| | | | | bound | | | systemic use | | | | | | |
| | | | | (Maraviroc) | | | | | | | | | |
| 4MQS | A | P08172 | CHRM2 | Already | Bethanechol | N07AB02 | Nervous | 0.0710384 | no | | no | | 1 |
| | | | | bound | | | system | | | | | | |
| | | | | (Iperoxo) | | | | | | | | | |
| 4NUA | A | P14324 | FDPS | 4UMJ | Ibandronic | M05BA06; | Musculoskeletal | 0.1496558 | no | 0.1777209 | no | | 2 |
| | | | | (Ibandronic | acid; | M05BA03; | system | | | | | | |
| | | | | acid); 4KPJ | Pamidronic | M05BA07; | | | | | | | |
| | | | | (Pamidronic | acid; | M05BA08 | | | | | | | |
| | | | | acid); 5CG5 | Risedronic | | | | | | | | |
| | | | | (Risedronic | acid; | | | | | | | | |
| | | | | acid); 2F8C | Zoledronic | | | | | | | | |
| | | | | (Zoledronic | acid | | | | | | | | |
| | | | | acid) | | | | | | | | | |
| 4PYI | A | P21964 | COMT | 4PYL | Tolcapone | N04BX01 | Nervous system | 0.3192671 | no | | no | | 0 |
| | | | | (Tolcapone) | | | | | | | | | |
| 4WZI | A | Q07343 | PDE4B | 3TVX | Pentoxifylline; | C04AD03; | Cardiovascular | 0.0971676 | no | | yes | 0.1008321 | 1 |
| | | | | (Pentoxifylline); | Roflumilast | R03DX07 | system; | | | | | | |
| | | | | 1XMU | | | Respiratory | | | | | | |
| | | | | (Roflumilast) | | | system | | | | | | |
| 4WZI | B | Q07343 | PDE4B | 3TVX | Pentoxifylline; | C04AD03; | Cardiovascular | 0.092898 | no | | yes | 0.1008321 | 1 |
| | | | | (Pentoxifylline); | Roflumilast | R03DX07 | system; | | | | | | |
| | | | | 1XMU | | | Respiratory | | | | | | |
| | | | | (Roflumilast) | | | system | | | | | | |
| 4Y86 | A | Q86WN6 | PDE9A | 3TVX | Pentoxifylline | C04AD03 | Cardiovascular | 0.2149452 | no | | yes | 0.1385589 | 2 |
| | | | | (Pentoxifylline) | | | system | | | | | | |
| 4YAY | A | P30556 | AGTR1 | 4ZUD | Olmesartan | C09CA08 | Cardiovascular | 0.1931762 | no | | no | | 1 |
| | | | | (Olmesartan) | medoxomil | | system | | | | | | |
| 5CJF | A | Q9ULX7 | CA14 | 3DD0 | Ethoxzolamide | *S01; | *Sensory organs; | 0.2118685 | no | | yes | 0.2118685 | 0 |
| | | | | (Ethoxzolamide) | | *C03; | *Cardiovascular | | | | | | |
| | | | | | | *N03 | system; | | | | | | |
| | | | | | | | *Nervous | | | | | | |
| | | | | | | | system | | | | | | |
| 5DC8 | A | Q86VC8 | HDAC8 | SEEN | Belinostat; | L01XX49; | Antineoplastic and | | no | | yes | | 0 |
| | | | | (Belinostat); | Panobinostat | L01XX42 | immunomodulating | | | | | | |
| | | | | 5EF8 | | | agents | | | | | | |
| | | | | (Panobinostat) | | | | | | | | | |
| 5DXU | A | O00329 | PIK3CD | 4XE0 | Idelalisib | L01XX47 | Antineoplastic and | 0.0717044 | no | | no | | 1 |
| | | | | (Idelalisib) | | | immunomodulating | | | | | | |
| | | | | | | | agents | | | | | | |
| 5EDM | A | Q9UCA1 | F2 | 4HFP | Argatroban; | B01AE03; | Blood and | 0.0993119 | no | | yes | 0.0937306 | 2 |
| | | | | (Argatroban); | Bivalirudin | B01AE06 | blood forming | | | | | | |
| | | | | 3VXF | | | organs | | | | | | |
| | | | | (Bivalirudin) | | | | | | | | | |
| 5F19 | A | P35354 | PTGS2 | 5IKQ | Meclofenamic | B01AE03; | Musculoskeletal | 0.1022602 | no | | yes | 0.0618429 | 1 |
| | | | | (Meclofenamic | acid | B01AE06 | system | | | | | | |
| | | | | acid) | | | | | | | | | |
| 5F19 | B | P35354 | PTGS2 | 5IKQ | Meclofenamic | M01AG04; | Musculoskeletal | 0.1040575 | no | | yes | 0.0618429 | 1 |
| | | | | (Meclofenamic | acid | M02AA18 | system | | | | | | |
| | | | | acid) | | | | | | | | | |
| 5FQD | B | Q96SW2 | CRBN | Already bound | Lenalidomide; | L04AX04; | Antineoplastic and | 0.152127 | no | | no | | 2 |
| | | | | (Lenalidomide); | Pomalidomide; | L04AX06; | immunomodulating | | | | | | |
| | | | | 4TZU | Thalidomide | L04AX02 | agents | | | | | | |
| | | | | (Pomalidomide); | | | | | | | | | |
| | | | | 4TZC | | | | | | | | | |
| | | | | (Thalidomide) | | | | | | | | | |
| 5FQD | C | Q96HD2 | CSNK1A1 | | | | | 0.0448169 | no | | yes | 0.0343741 | 1 |
| 5I6X | A | P31645 | SLC6A4 | Already bound | Paroxetine; | N06AB05; | Nervous system | 0.1719944 | no | | no | | 2 |
| | | | | (Paroxetine); | Citalopram | N06AB04 | | | | | | | |
| | | | | 5171 | | | | | | | | | |
| | | | | (Citalopram) | | | | | | | | | |

### Example 7—Comparison of 3DTS and CADD Score for Determining Mutation Tolerant Amino Acids in PPARG

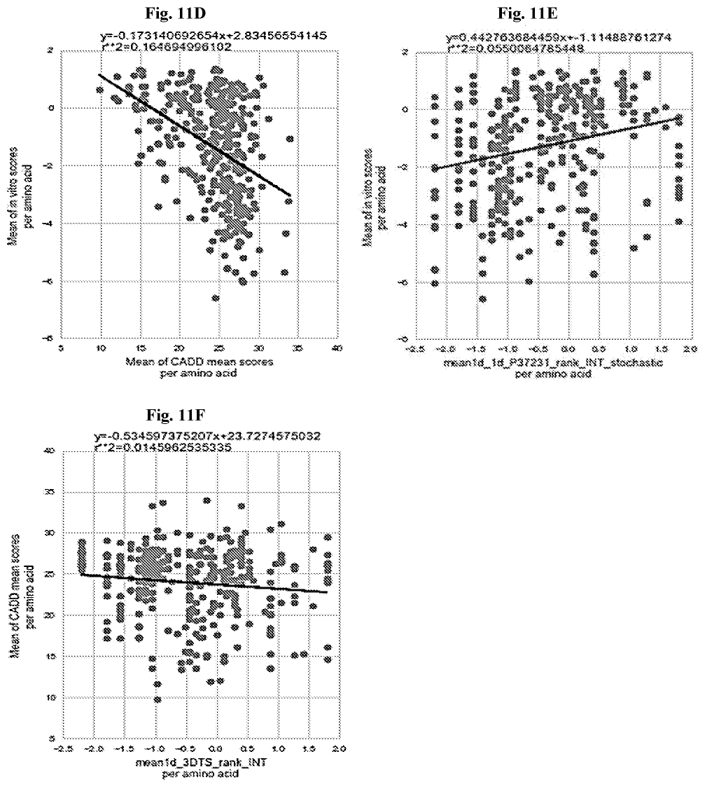

FIG. 11 shows comparisons between 3DTS and methods using in vitro experimental data (example 2), or the CADD score. FIGS. 11A-11F shows a comparison of 3DTS, CADD and in vitro functional data for PPARG. FIGS. 11A-11C represent feature level comparisons while FIGS. 11D-11F represent amino acid position level comparisons. CADD and 3DTS show low correlation between the two methods both at the feature level (FIG. 11C, r²=0.05) and the amino acid level (FIG. 11F, r²=0.015) suggesting the two metrics pick up different types of information. A strong correlation (FIG. 11B, r²=0.47) is seen between 3DTS and the in vitro functional data when comparing feature level scores (FIG. 11A, r²=0.16) compared to amino acid level scores (FIG. 11E, r²=0.06). CADD shows low correlation (FIG. 11D, r²=0.16) at the amino acid positional level, and fails to discriminate functional and non-functional variations at higher CADD scores. CADD scores below 15 show many benign (non-functional) variants as determined by the prospective functional in vitro assay (average score <−2 is considered intolerant). However, as CADD scores increase, the ability to discriminate between these functional and non-functional PPARG variants becomes more difficult. 3DTS values shows strong correlation with the in vitro data.

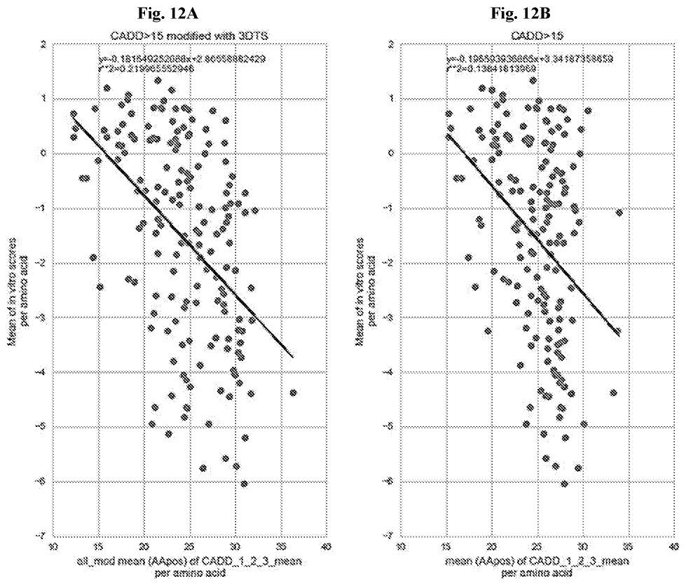

As there is effectively no correlation between 3DTS and CADD scores, we sought to combine these two metrics to improve prediction of the functional consequences of variants. We demonstrate improvement for CADD scores >15 when combined with 3DTS. FIGS. 12A and 12B show that 3DTS improves discrimination of functional and benign missense variation for PPARG (FIG. 12A, r²=0.22), compared to CADD score alone (FIG. 12B, r²=0.14). Empirically determined modifiers of the mean positional CADD score were determined from a training set using the rank-based inverse transformed mean score. The results of a test set are shown, with an increased correlation with in vitro functional scores observed using the modified score.

### CADD Score Calculation

Combined annotation dependent depletion (CADD) scores, a tool for scoring the deleteriousness of genetic variants were included if single nucleotide variation resulted in an amino acid change. For FIGS. 11A-11F, CADD scores were averaged across a nucleotide, then across a codon (amino acid position), and finally across a 3DTS-defined feature. Functional in vitro scores were averaged across an amino acid position, then across a 3DTS-defined feature. The 3DTS scores were subjected to a rank-based inverse transformation; with amino acid positions within a feature also being assigned, the transformed 3DTS score.

For FIGS. 12A and 12B, mean CADD scores greater than 15 were retained (331 amino acid positions) and this set was randomly divided into a training (165 amino acid positions) and a test set (166 amino acid positions). CADD scores were empirically modified dependent upon the transformed 3DTS score. The best linear correlation with the mean functional in vitro data determined the CADD modification parameters. The CADD scores in the test set was then modified with these parameters and a comparison was made with the unmodified CADD scores.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Throughout this disclosure, various patents, patent applications and publications are referenced. The disclosures of these patents, patent applications, accessioned information (e.g., as identified by PUBMED, UNIPROT, PDB, or EBI accession numbers) and publications in their entireties are incorporated into this disclosure by reference in order to more fully describe the state of the art as known to those skilled therein as of the date of this disclosure. The following electronic documents, including source codes, are incorporated by reference herein in their entirety: doi(dot)org/10.5281/zenodo.1311198; and github(dot)com/pityka/3DTS, which are viewable using the interactive browser from protc(dot)labtelenti(dot)org.

This disclosure will govern in the instance that there is any inconsistency between the patents, patent applications and publications cited and this disclosure.

What is claimed is:

1. A computer readable medium comprising computer-executable instructions, which, when executed by a processor, cause the processor to carry out a method or a set of steps for determining a tolerance or intolerance of one or more amino acids of a protein to a variation, the method or steps comprising,
a) determining a likelihood of observing missense variation, given a first selective pressure, from a genetic variation data of a plurality of individuals and features from 3D protein structures and/or models;
b) determining a posterior distribution on the selective pressure using step (a);
c) determining a second selective pressure on 3D features of the protein by determining the mean of the posterior distribution, wherein the mean is used to determine a three-dimensional tolerance score (3DTS); and
d) determining the tolerance of one or more amino acids of a protein to a variation based on the 3DTS.

2. A computer readable medium comprising computer-executable instructions, which, when executed by a processor, cause the processor to carry out a method or a set of steps for determining druggability of a protein, the method or steps comprising,
a) determining a likelihood of observing missense variation, given a first selective pressure, from a genetic variation data of a plurality of individuals and features from 3D protein structures and/or models;
b) determining a posterior distribution using step (a); and
c) determining a second selective pressure on 3D features of the protein by determining the mean of the posterior distribution, wherein the mean is used to determine a three-dimensional tolerance score (3DTS); and
d) determining the protein as being druggable if one or more amino acids in the protein is determined as being intolerant to the variation based on the 3DTS.

3. A computer readable medium comprising computer-executable instructions, which, when executed by a processor, cause the processor to carry out a method or a set of steps for determining drug resistance potential of a variant protein, the method or steps comprising,
a) determining a likelihood of observing missense variation, given a first selective pressure, from a genetic variation data of a plurality of individuals and features from 3D protein structures and/or models;
b) determining a posterior distribution using step (a); and
c) determining a second selective pressure on 3D features of the variant protein by determining the mean of the posterior distribution, wherein the mean is used to determine a three-dimensional tolerance score (3DTS); and
d) determining the variant protein as being potentially drug resistant if one or more amino acids in the protein is determined as being tolerant to the variation based on the 3DTS.

4. The computer readable media of any one of claims 1-3, wherein the likelihood function comprises (a) a background mutation rate, (b) a fraction of single nucleotide changes that result in an amino acid change, (c) a number of individuals or samples with called nucleotides, and (d) an adjustment factor, whose estimate serves as a tolerance score.

5. The computer readable media of claim 4, wherein the background mutation rate is estimated using genetic data and a reference genome.

6. The computer readable media of claim 4, wherein the background mutation rate is estimated using the synonymous variation rate.

7. The computer readable media of claim 4, wherein the background mutation rate may be estimated using the intergenic variation rate

8. The computer readable media of claim 7, wherein the intergenic variation rate may be estimated genome-wide

9. The computer readable media of claim 8, wherein the intergenic variation rate may be estimated specific to a chromosome

10. The computer readable media of any one of claims 4 to 9, wherein the background mutation rate may vary on a per nucleotide basis dependent upon the nucleotide's context.

11. The computer readable media of claim 8, wherein the nucleotide context comprises a heptamer representing 3 nucleotides up and downstream of a reference nucleotide.

12. The computer readable media of claim 5, wherein the fraction of single nucleotide changes that result in an amino acid change include amino acid changes that result in significant physiochemical changes.

13. The computer readable media of claim 4, where the background mutation rates are estimated by maximizing the likelihood fixing the s parameter to 1.

14. The computer readable media of claim 4, wherein the likelihood function is evaluated as the sum of Bernoulli trials over the loci corresponding to the 3D feature.

15. The computer readable media of claim 14, wherein each Bernoulli trial represents an individual's variation information at a given locus/nucleotide.

16. The computer readable media of claim 15, wherein the sum of Bernoulli trials results in a binomial distribution comprising a Poisson approximation.

17. The computer readable media of claim 16, wherein the Poisson approximation estimates the probability of observing at least one missense mutation in the 3D feature using Le Cam's approximation.

18. The computer readable media of claim 1, wherein the likelihood function is combined with a prior distribution to produce a posterior distribution representing the probabilities of a selective pressure on a 3D locus.

19. The computer readable media of claim 18, wherein the mean of the posterior distribution represents a 3D Tolerance Score (3DTS).

20. The computer readable media of claim 1, wherein the protein structure or model is representative of an X-ray crystal structure, an NMR structure, a CRYOEM structure.

21. The computer readable media of claim 1, wherein the protein structure or model is representative of a similarity model, a homology model, an ab initio model.

22. The computer readable media of any one of claims 1 to 21, wherein an intolerant feature is defined as a 3DTS value between the 0^{th} and the 20^{th} percentile of all 3DTS scores for the proteome; or wherein a tolerant feature is defined as a 3DTS value between the 50^{th} and the 100^{th} percentile of all 3DTS scores for the proteome.

23. The computer readable media of claim 22, wherein the proteome comprises at least 1000 proteins, particularly at least 5000 proteins, more particularly at least 10000 proteins, especially at least 20000 proteins, and specifically all the proteins of the proteome of a subject which encodes the protein.

24. The computer readable media of any one of claims 1 to 19, wherein an intolerant feature is defined as the lowest ranked 3DTS values within a protein; or wherein a tolerant feature is defined as the highest ranked 3DTS values within a protein.

25. The computer readable media of claim 24, wherein the lowest rank 3DTS values include the bottom 25%, particularly bottom 10%, more particularly bottom 5% and especially bottom 2% of all ranked 3DTS values within a protein.

26. The computer readable media of any one of claims 1-3, wherein the posterior distribution on the selective pressure is determined using a likelihood function and/or assuming a uniform prior function.

27. A system for determining a tolerance or intolerance of one or more amino acids of a protein to a variation, comprising,
a) a background module for determining a likelihood of observing missense variation, given a first selective pressure, from a genetic variation data of a plurality of individuals and features from 3D protein structures and/or models;
b) a distribution module for determining a posterior distribution using step (a); and
c) a scoring module for determining a second selective pressure on 3D features of the protein by determining the mean of the posterior distribution, wherein the mean is used to determine a three-dimensional tolerance score (3DTS) and wherein the 3DTS is used to determine the tolerance or intolerance of one or more amino acids of a protein to a variation.

28. A system for determining druggability of a protein, comprising,
a) a background module for determining a likelihood of observing missense variation, given a first selective pressure, from a genetic variation data of a plurality of individuals and features from 3D protein structures and/or models;
b) a distribution module for determining a posterior distribution using step (a); and
c) a scoring module for determining a second selective pressure on 3D features of the protein by determining the mean of the posterior distribution, wherein the mean is used to determine a three-dimensional tolerance score (3DTS) and wherein the 3DTS is used to determine the druggability of the protein.

29. A system for determining drug resistance potential of a variant protein, comprising,
a) a background module for determining a likelihood of observing missense variation, given a first selective pressure, from a genetic variation data of a plurality of individuals and features from 3D protein structures and/or models;
b) a distribution module for determining a posterior distribution using step (a); and
c) a scoring module for determining a second selective pressure on 3D features of the variant protein by determining the mean of the posterior distribution, wherein the mean is used to determine a three-dimensional tolerance score (3DTS) and wherein the 3DTS is used to determine the drug resistance potential of the variant protein.

30. The system of any one of claims 27-29, wherein the posterior distribution on the selective pressure is determined using a likelihood function and/or assuming a uniform prior function.

31. A method of determining a three-dimensional tolerance score (3DTS) for one or more amino acids of a protein comprising:
a) determining a likelihood of observing missense variation, given a first selective pressure, from a genetic variation data of a plurality of individuals and features from 3D protein structures and/or models;
b) determining a posterior distribution using step (a);
c) determining a second selective pressure on 3D features of the protein by determining the mean of the posterior distribution, wherein the mean is used to determine the 3DTS; and
d) determining the tolerance of one or more amino acids of a protein to a variation based on the 3DTS.

32. A method of determining druggability of a protein, comprising,
a) determining a likelihood of observing missense variation, given a first selective pressure, from a genetic variation data of a plurality of individuals and features from 3D protein structures and/or models;
b) determining a posterior distribution using step (a); and
c) determining a second selective pressure on 3D features of the protein by determining the mean of the posterior distribution, wherein the mean is used to determine a three-dimensional tolerance score (3DTS); and
d) determining the protein as being druggable if one or more amino acids in the protein is determined as being intolerant to the variation based on the 3DTS.

33. A method of determining drug resistance potential of a variant protein, comprising,
a) determining a likelihood of observing missense variation, given a first selective pressure, from a genetic variation data of a plurality of individuals and features from 3D protein structures and/or models;
b) determining a posterior distribution using step (a); and
c) determining a second selective pressure on 3D features of the variant protein by determining the mean of the posterior distribution, wherein the mean is used to determine a three-dimensional tolerance score (3DTS); and
d) determining the variant protein as being potentially drug resistant if one or more amino acids in the protein is determined as being tolerant to the variation based on the 3DTS.

34. The method of any one of claims 31-33, wherein the posterior distribution on the selective pressure is determined using a likelihood function and/or assuming a uniform prior function.

35. The method of any one of claims 31-33, wherein the likelihood function contains terms defining a background mutation rate, a fraction of single nucleotide changes that result in an amino acid change, a number of individuals with called nucleotides, and an adjustment factor, whose estimate serves as a tolerance score

36. The method of claim 35, wherein the background mutation rate is estimated using genetic data and a reference genome

37. The method of claim 35, wherein the background mutation rate is estimated using the synonymous variation rate

38. The method of claim 35, wherein the background mutation rate is estimated using the intergenic variation rate

39. The method of claim 38, wherein the intergenic variation rate may be estimated genome-wide

40. The method of claim 38, wherein the intergenic variation rate may be estimated specific to a chromosome

41. The methods of claims 35, wherein the background mutation rate may vary on a per nucleotide basis dependent upon the nucleotide's context

42. The method of claim 41, wherein the nucleotide context can be a heptamer representing 3 nucleotides up and downstream of a reference nucleotide.

43. The method of claim 35, wherein the fraction of single nucleotide changes that may result in an amino acid change may be modulated dependent on those amino acid changes that may result in significant physiochemical changes.

44. The methods of claim 35 where the background mutation rates are estimated by maximizing the likelihood fixing the s parameter to 1.

45. The methods of claim 44, wherein the likelihood function is evaluated as the sum of Bernoulli trials over the loci corresponding to the 3D feature.

46. The method of claim 45, wherein each Bernoulli trial represents an individual's variation information at a given locus/nucleotide.

47. The method of claim 45 or 46, wherein the sum of Bernoulli trials results in a binomial distribution comprising a Poisson approximation.

48. The method of claim 47, wherein the Poisson approximation estimates the probability of observing at least one missense mutation in the 3D feature using Le Cam's approximation.

49. The method of claim 48, wherein the likelihood function may be combined with a prior distribution to produce a posterior distribution representing the probabilities of a selective pressure on a 3D locus.

50. The method of claim 49, wherein the mean of the posterior distribution may represent a 3D tolerance Score (3DTS)

51. The method of any one of claims 31-33, wherein the protein structure or model represents an X-ray crystal structure, an NMR structure, a CRYOEM structure or a combination thereof.

52. The method of any one of claims 31-33, wherein a model represents a homology model, an ab initio model or a combination thereof.

53. The method of any one of claims 31 to 52, wherein an intolerant feature is defined as a 3DTS value between the 0^{th} and the 20^{th} percentile of all 3DTS scores for the proteome.

54. The method of any one of claims 31 to 52, wherein an intolerant feature is defined as the lowest 3DTS values within a protein.

55. The method of any one of claims 31 to 33, wherein step (a) comprises determining a synonymous global mutation rate, defined as parameter p, which is the expected number of mutations at a locus assuming all mutations at a locus are neutral.

56. The method of any one of claims 31 to 33, wherein step (a) comprises determining a synonymous local mutation rate, which estimates heterogeneity across the genome, but is only evaluated on a single amino acid chain of the protein.

57. The method of any one of claims 31 to 33, further comprising determining an intergenic variation rate.

58. The method of claim 572, wherein the intergenic variation rate comprises a global intergenic variation rate or a chromosome-specific intergenic variation rate.

59. The method of any one of claims 31 to 33, wherein step (b) comprises determining a propensity towards missense variation.

60. The method of claim 59, wherein the propensity towards missense variation for a nucleotide is determined as a statistical probability of a single nucleotide variation leading to a missense variant of the protein (parameter b), based on the protein isoform for the 3D structure, the transcript encoding the protein isoform and the reference genome encoding the transcript for the locus.

61. The method of any one of claims 31 to 33, wherein step (c) comprises determining tolerance to missense variation, which is defined by the mean of a posterior distribution, calculated through numerical integration using the Gauss-Legendre quadrature or estimated by importance sampling.

62. The method of claim 61, wherein step (c) comprises determining a mean of the posterior distribution by combining a prior distribution which assumes all missense variants are tolerant and is set as a uniform distribution and likelihood function which is defined as the sum of a series of Bernoulli trials.

63. The method of any one of claims 31 to 33, wherein step (c) comprises implementing a machine learning algorithm.

64. A method of identifying druggability of a protein comprising:
a) determining a global mutation rate, wherein the global mutation rate is an expected probability of any given nucleotide of the protein to vary;
b) determining a variant specific mutation rate for a missense mutation of a nucleic acid encoding the one or more amino acids of the protein, wherein the variant specific mutation rate is an observed probability of the missense mutation to occur in a sample nucleotide data set; and
c) determining the one or more amino acids of the protein as intolerant to variation if the variant specific mutation rate is less than the global mutation rate; and
d) identifying the protein as being druggable if one or more amino acids in the protein is determined as being intolerant to the variation.

65. The method of claim 64, wherein the amino acid is determined as being intolerant based on a rank based metric or a percentile metric.

66. The method of claim 65, wherein the rank based metric or percentile metric is determined in relation to a proteome comprising at least 5K proteins, at least 10K proteins, at least 15K proteins or the entire proteome of a subject.

67. The method of claim 64, wherein the one or more amino acids of the protein that are intolerant to variation comprises a binding pocket.

68. The method of claim 64, wherein the binding pocket comprises an active site, an allosteric site, an epitope, a cofactor binding site, or a prosthetic group binding site, or a combination thereof.

69. The method of claim 64, wherein the drug includes a small molecule or a large molecule.

70. The method of claim 69, wherein the small molecule is a compound having a molecular weight less than 5 kDa selected from an amino acid, a nucleic acid, an LNA, a PNA, a carbohydryate, a sugar, a lipid, a steroid, a biometal, a vitamin, a terpene, or a polymer thereof.

71. The method of claim 69, wherein the large molecule is a compound having a molecular weight greater than 5 kDa selected from an antibody, a hormone, a growth factor, a cytokine, or a combination thereof.

72. The method of claim 64, wherein the druggable protein is an enzyme, an antigen, or a receptor.

73. The method of claim 64, wherein the drug is an enzyme activator or inhibitor; an allosteric modulator; an agonist, a partial agonist or an antagonist; or an antibody.

74. A method of identifying a drug resistance potential of a variant protein comprising:
a) determining a global mutation rate, wherein the global mutation rate is an expected probability of any given nucleotide of the protein to vary;
b) determining a variant specific mutation rate for a missense mutation of a nucleic acid encoding the one or more amino acids of the protein, wherein the variant specific mutation rate is an observed probability of the missense mutation to occur in a sample nucleotide data set; and
c) determining the one or more amino acids of the protein as intolerant to variation if the variant specific mutation rate is less than the global mutation rate; and
d) identifying the protein as being drug resistant if one or more amino acids in the variant protein is determined as being tolerant to the variation compared to the one or more amino acids in a wild-type protein.

75. The method of claim 74, wherein the drug includes a small molecule or a large molecule.

76. The method of claim 74, wherein the small molecule is a compound of less than 5 kDa selected from an amino acid, a nucleic acid, an LNA, a PNA, a carbohydryate, a sugar, a lipid, a steroid, a biometal, a vitamin, a terpene, or a polymer thereof.

77. The method of claim 74, wherein the large molecule is a compound having a molecular weight greater than 5 kDa selected from an antibody, a hormone, a growth factor, a cytokine, or a combination thereof.

78. The method of claim 74, wherein the variant protein is potentially resistant to an antibiotic, an anticancer agent, a xenobiotic, an antagonist, an agonist or an allosteric modulator.

79. The method of claim 74, wherein the variant protein is potentially resistant to binding of an antibody or a ligand.

80. A method of determining a three-dimensional tolerance score (3DTS) for one or more amino acids of a protein comprising:
a) determining a global mutation rate, wherein the global mutation rate is an expected probability of any given nucleotide of the protein to vary;
b) determining a variant specific mutation rate for a missense mutation of a nucleic acid encoding the one or more amino acids of the protein, wherein the variant specific mutation rate is an observed probability of the missense mutation to occur in a sample nucleotide data set; and
c) determining the one or more amino acids of the protein as intolerant to variation if the variant specific mutation rate is less than the global mutation rate.

81. The method of claim 80, wherein the one or more amino acids of the protein comprise a plurality of amino acids.

82. The method of claim 81, wherein the plurality of amino acids comprises a protein feature or domain.

83. The method of claim 80, wherein the protein feature is selected from the list consisting of: an active site, a metal binding site, a chemical binding site, a DNA binding site, a nucleotide binding site, a zinc finger, a calcium binding site, a transmembrane domain, an intra membrane domain, a lipidation site, a glycosylation site, a phosphorylation site, a coiled-coil, an alpha helix, and a beta strand.

84. The method of any one of claims 80-83, wherein the global mutation rate is the mutation rate of the nucleotides encoding the protein, an intronic sequence of the protein, a 3′ untranslated region of the protein, a 5′ untranslated region of the protein, or any combination thereof.

85. The method of any one of claims 80-84, wherein the global mutation rate is the mutation rate for an entire human genome.

86. The method of any one of claims 80-85, wherein the global mutation rate is between about 1×10⁻⁶ and 5×10⁻⁶.

87. The method of any one of claims 80-86, wherein the global mutation rate is about 2.5×10⁻⁶.

88. The method of any one of claims 80-87, wherein the sample nucleotide data set comprises at least 1,000 different nucleic acid sequences from at least 1,000 different individuals encoding the protein.

89. The method of any one of claims 80-88, wherein the sample nucleotide data set comprises at least 10,000 different nucleic acid sequences from at least 10,000 different individuals encoding the protein.

90. The method of any one of claims 80-89, wherein the nucleotide data set comprises DNA.

91. The method of any one of claims 80-90, comprising determining the one or more amino acids of the protein as intolerant to variation if the variant specific mutation rate is 2 times less than the global mutation rate.

92. The method of any one of claims 80-91, comprising determining the one or more amino acids of the protein as intolerant to variation if the variant specific mutation rate is 5 times less than the global mutation rate.

93. The method of any one of claims 80-92, wherein the missense mutation is a hypothetical mutation.

94. The method of any one of claims 80-93, further comprising rendering a graphic representation of the protein with a visual indication of amino acids of the protein that are intolerant to variation.

95. The method of claim 94, wherein the graphic representation of the protein is three-dimensional.

96. The method of claim 95, wherein the graphic representation of the protein is rotatable around an x, y, or z axis.

97. The method of claim 96, wherein the graphic representation of the protein is reflectable across an x, y, or z axis.

98. A modulator that binds to any of the one or more amino acids of the protein that are intolerant to variation according to the method of claims 31-97.

99. The modulator of claim 98, wherein the modulator is an antibody or antigen binding fragment thereof.

100. The modulator of claim 98, wherein the modulator binds at a non-active or an allosteric site.

101. A computer-implemented system comprising a digital processing device comprising at least one processor, an operating system configured to perform executable instructions, a memory, and a computer program including instructions executable by the digital processing device to create an application comprising:
a) a software module determining a global mutation rate, wherein the global mutation rate is an expected probability of any given nucleotide of the protein to vary;
b) a software module determining a variant specific mutation rate for a missense mutation of a nucleic acid encoding the one or more amino acids of the protein, wherein the variant specific mutation rate is an observed probability of the missense mutation to occur in a sample nucleotide data set; and
c) a software module determining the one or more amino acids of the protein as intolerant to variation if the variant specific mutation rate is less than the global mutation rate.

102. The system of claim 101, wherein the one or more amino acids of the protein comprise a plurality of amino acids.

103. The system of claim 102, wherein the plurality of amino acids comprises a protein feature or domain.

104. The system of claim 103, wherein the protein feature or domain is selected from the list consisting of: an active site, a metal binding site, a chemical binding site, a DNA binding site, a nucleotide binding site, a zinc finger, a calcium binding site, a transmembrane domain, an intra membrane domain, a lipidation site, a glycosylation site, a phosphorylation site, a coiled-coil, an alpha helix, and a beta strand.

105. The system of any one of claims 101-104, wherein the global mutation rate is the mutation rate of the nucleotides encoding the protein, an intronic sequence of the protein, a 3′ untranslated region of the protein, a 5′ untranslated region of the protein, or any combination thereof.

106. The system of any one of claims 101-105, wherein the global mutation rate is the mutation rate for an entire human genome or for a protein-encoding portion of a human genome.

107. The system of any one of claims 101-106, wherein the global mutation rate is between about 1×10⁻⁶ and 5×10⁻⁶.

108. The system of any one of claims 101-107, wherein the global mutation rate is about 2.5×10⁻⁶.

109. The system of any one of claims 101-108, wherein the sample nucleotide data set comprises at least 1,000 different nucleic acid sequences from at least 1,000 different individuals encoding the protein.

110. The system of any one of claims 101-109, wherein the sample nucleotide data set comprises at least 10,000 different nucleic acid sequences from at least 10,000 different individuals encoding the protein.

111. The system of any one of claims 101-110, wherein the nucleotide data set comprises DNA.

112. The system of any one of claims 101-111, comprising determining the one or more amino acids of the protein as intolerant to variation if the variant specific mutation rate is 2 times less than the global mutation rate.

113. The system of any one of claims 101-112, comprising determining the one or more amino acids of the protein as intolerant to variation if the variant specific mutation rate is 5 times less than the global mutation rate.

114. The system of any one of claims 101-113, wherein the missense mutation is a hypothetical mutation.

115. The system of any one of claims 101-114, further comprising rendering a graphic representation of the protein with a visual indication of amino acids of the protein that are intolerant to variation.

116. The system of claim 115, wherein the graphic representation of the protein is three-dimensional.

117. The system of claim 116, wherein the graphic representation of the protein is rotatable around an x, y, or z axis.

118. The system of claim 117, wherein the graphic representation of the protein is reflectable across an x, y, or z axis.

119. An antagonist that binds to any of the one or more amino acids of the protein that are intolerant to variation according to the system of any one of claims 101 to 118.

120. The antagonist of claim 119, wherein the antagonist is an antibody or antigen binding fragment thereof.

121. The antagonist of claim 119, wherein the antagonist binds at a non-active or an allosteric site.
